SEQUENCE LISTING (48 sequences)

SEQ ID NO: 1 - DNA, 2235 bp, Artificial Sequence
  Description of Artificial Sequence Synthetic polynucleotide
atggacgcggaggctagcgatactcacttttttgctcactccaccttaaaggcagatcgcagcgattggcagcctctggtcgagcatctacaggctgttgcccgtttggcaggagagaaggctgccttcttcggcggcggtgaattagctgctcttgctggtctgttgcatgacttgggtaaatacactgacgagtttcagcggcgtattgcgggtgatgccatccgtgtcgatcactctactcgcggggccatactggcggtagaacgctatggcgcgctaggtcaattgctagcctacggcatcgctggccaccatgccgggttggccaatggccgcgaggctggtgagcgaactgccttggtcgaccgcctgaaaggggttgggctgccacggttattggaggggtggtgcgtggaaatcgtgctacccgagcgccttcaaccaccgccactaaaagcgcgcctggaaagaggtttctttcagttggcctttcttggccggatgctcttttcctgcttggttgatgcggattatctagataccgaagccttctaccaccgcgtcgaaggacggcgctcccttcgcgagcaagcgcggccgaccttggccgagttacgcgcagcccttgatcggcatctgactgagttcaagggagatacgccggtcaaccgcgttcgcggggagatattggccggcgtgcgcggcaaggcgagcgaacttcccgggctgttttctctcacagtgcccacaggaggcggcaagaccctggcctctctggctttcgccctggatcacgctctagctcatgggctgcgccgggtgatctacgtgattcccttcactagcatcgtcgagcagaacgctgcggtattccgtcgtgcactcggggccttaggcgaagaggcggtgctggagcatcacagcgccttcgttgatgaccgccggcagagcctggaggccaagaagaaactgaacctagcgatggagaactgggacgcgcctatcgtggtgaccactgcagtgcagttcttcgaaagcctgtttgccgaccgtccagcccagtgccgcaagctacacaacatcgccggcagcgtggtgattcttgacgaggcacagaccctaccgctcaagctgttgcggccctgcgttgccgcccttgatgaactggcgctcaactaccgttgtagcccagttctctgtactgccacgcagccagcgcttcaatcgccggatttcatcggtgggctgcaggacgtacgtgagctggcgcccgagccgcagcggctgttccgggagttggtgcgggtacgaatacggacattgggcccgctcgaagatgcggccttgactgagcagatcgccaggcgtgaacaagtgctgtgcatcgtcaacaatcgacgccaggcccgtgcgctctatgagtcgcttgccgagttgcccggtgcccgccatctcaccaccctgatgtgcgccaagcaccgtagcagcgtgctggccgaggtgcgccagatgctcaaaaagggggagccctgtcgcctggtggccacctcgctgatcgaggccggtgtggatgtggattttcccgtggtactgcgtgccgaggctggattggattccatcgcccaggccgcgggacgctgcaatcgcgaaggcaagcggccgctggccgaaagcgaggtgctggtgttcgccgcggccaattctgactgggcgccacccgaggaactcaagcagttcgcccaggccgcccgcgaagtgatgcgcctgcacccggatgattgcctgtccatggcggccatcgagcggtattttcgcatactgtactggcagaagggcgcggaggagttggatgcgggtaacctgctcggcctgattgagagaggccggctcgatggcctgccctacgagactttggccaccaagttccgcatgatcgacagccttcaactgccggtgatcatcccatttgatgacgaggccagagcagccctgcgcgagctggagttcgccgacggctgcgccgccatcgcccgtcgcctgcagccatatctggtgcagatgccacgcaagggttatcaggcattgcgggaagccggtgcgatccaggcggcggcaggtacgcgttatggtgagcagtttatggcgttggtcaaccctgatctgtatcaccaccaattcgggttgcactgggataatccggcctttgtcagcagcgagcggctatgttggtag

SEQ ID NO: 2 - DNA, 675 bp, Artificial Sequence
  Description of Artificial Sequence Synthetic polynucleotide
atggcctacggaattcgcttaatggtctggggcgagcgtgcctgcttcacccgcccggaaatgaaggtggaacgcgtctcttacgatgcgatcacgccgtccgccgcgcgcggcattctcgaggctatccactggaagccggcgattcgctgggtggtggatcgcattcaagtgcttaagccgatccgcttcgaatccatccggcgcaacgaggtcggcggcaagctgtccgctgtcagcgtcggtaaggcaatgaaggccgggcgtactaatggtctggtgaatctggtcgaggaggatcgccagcagcgcgcgactactctgctgcgcgatgtctcctatgtcatcgaggcgcatttcgagatgactgacagggctggcgccgacgatacggtgggcaagcatctggatatcttcaaccgtcgcgcacggaaggggcagtgcttccatacaccctgcctaggcgtgcgcgagtttccggccagttttcggttgctggaagagggcagtgccgagcctgaagtcgatgcctttctgcgcggcgagcgtgatctgggctggatgctgcatgacattgacttcgccgatggcatgaccccgcacttcttccgtgccctgatgcgcgatgggctgatcgaggtgccggccttcagggcggcagaggacaaggcatga

SEQ ID NO: 3 - DNA, 1764 bp, Artificial Sequence
  Description of Artificial Sequence Synthetic polynucleotide
atgatcctttcggccctcaatgactattatcagcgactgctggagcggggtgaagcgaatatctcacccttcggctacagccaagaaaagatcagttacgccctgctgctgtccgcacaaggagagttgctggacgtgcaggacattcgcttgctctctggcaagaagcctcaacccaggcttatgagtgtgccgcagccggagaagcgcacctcgggcatcaagtccaacgtactgtgggacaagaccagctatgtgctgggtgttagtgccaagggcggagagcgtactcagcaggagcacgagtccttcaagacgctgcaccggcagatcttggttggggaaggcgaccccggtctgcaggccttgctccagttcctcgactgttggcagccggagcagttcaagcccccgctgttcagcgaagcaatgctcgacagcaacttagtgttccgcctagacggccaacaacgctatctgcacgagactccggcggccctggcgttgcgtacccggctgttggccgacggcgacagccgcgaggggctgtgcctagtctgcggccaacgtcagccgttggcgcgcctgcatccagcggtcaagggcgtcaatggtgcccagagttcgggggcttccatcgtctccttcaacctcgacgctttttcctcctacggcaagagccagggggaaaatgctccggtctccgaacaggccgcctttgcctacaccacggtgctcaaccatttgttgcgtcgcgacgagcacaaccgccagcgcctgcagattggcgacgcgagtgtggtgttctgggcgcaggcggatactcctgctcaggtggccgccgccgagtcgaccttctggaacctgctggagccacccgcagatgatggtcaggaagcggaaaagctgcgcggcgtgctggatgctgtggccacggggcggcccttgcatgagctcgactcgctaatggaggaaggtacccgcatttttgtgttagggctggcgcccaatacctcgcgactgtccattcggttctgggcagtcgatagccttgcggtattcacccagcatctggccgagcatttccgggatatgcaccttgagcctctgccctggaagacggagccggccatctggcgcttgctctatgctaccgcgcccagtcgtgacggcagagccaagaccgaagacgtactcccacaactggccggtgaaatgacccgcgccatcctgaccggcagccgctatccgcgcagtttgctagccaacctgatcatgcgcatgcgtgccgacggcgacgtctctggcatacgcgtcgcgctgtgcaaggccgtgctcgctcgcgaggcacgcctgagcggcaaaattcaccaagaggagctacctatgagtctcgacaaggacgccagcaaccccggctatcgcttggggaggctgttcgccgtgttggaaggcgcccagcgcgcagccctgggcgacagggtcaatgccactatccgtgaccgctactacggtgccgcgtccagcacgccagccacggttttcccgatactgctgcgcaacacacaaaaccacttggccaagctgcgcaaggagaagcccggactagcagtgaacctagagcgcgatataggcgaaatcattgacggtatgcagagccaattcccgcgttgcctgcgcctggaggaccagggacgctttgctattggttactaccaacaggcccaggcccgtttcaaccgtggccccgattccgtcgagtaa

SEQ ID NO: 4 - DNA, 870 bp, Artificial Sequence
  Description of Artificial Sequence Synthetic polynucleotide
atgaccgccatctccaaccgctacgagttcgtttacctctttgatgtcagcaatggcaatcccaatggcgacccggatgctggcaacatgccgcgtctcgatccggaaaccaaccaggggttggtcactgacgtttgcctcaagcgcaagatccgcaactacgtcagcctggagcaggaaagtgcccccggctatgccatctatatgcaggaaaaatccgtgctgaataaccagcacaaacaggcctacgaggcgctcggtatcgagtcagaggcaaagaaactgcccaaggacgaagccaaggcgcgcgaactgacctcttggatgtgcaagaacttcttcgatgtgcgtgctttcggggcggtgatgaccaccgagattaatgccggccaggtgcgtggaccgatccaactggcattcgccacgtctatcgacccggtattgcctatggaggtatccatcacccgcatggcggtgactaacgaaaaggatttggagaaggaacgcaccatgggacgcaagcacatcgtgccttacggcttgtaccgcgcccatggtttcatctctgccaagttggccgagcgaaccggcttttccgacgacgacttggaactgctatggcgcgctttggccaatatgttcgaacacgaccgctcggcggcacgtggcgagatggcagcgcgcaagttgatcgtcttcaagcatgagcatgccatgggcaatgcacccgcccatgtgctgttcggcagcgttaaggtcgagcgagtcgagggggacgcagttacaccagcacgcggtttccaggattaccgtgtcagcatcgatgcggaagctctgcctcagggcgtgagcgtgcgcgagtacctctag

SEQ ID NO: 5 - protein, 744 aa, Artificial Sequence
  Description of Artificial Sequence Synthetic polypeptide
MDAEASDTHFFAHSTLKADRSDWQPLVEHLQAVARLAGEKAAFFGGGELAALAGLLHDLGKYTDEFQRRIAGDAIRVDHSTRGAILAVERYGALGQLLAYGIAGHHAGLANGREAGERTALVDRLKGVGLPRLLEGWCVEIVLPERLQPPPLKARLERGFFQLAFLGRMLFSCLVDADYLDTEAFYHRVEGRRSLREQARPTLAELRAALDRHLTEFKGDTPVNRVRGEILAGVRGKASELPGLFSLTVPTGGGKTLASLAFALDHALAHGLRRVIYVIPFTSIVEQNAAVFRRALGALGEEAVLEHHSAFVDDRRQSLEAKKKLNLAMENWDAPIVVTTAVQFFESLFADRPAQCRKLHNIAGSVVILDEAQTLPLKLLRPCVAALDELALNYRCSPVLCTATQPALQSPDFIGGLQDVRELAPEPQRLFRELVRVRIRTLGPLEDAALTEQIARREQVLCIVNNRRQARALYESLAELPGARHLTTLMCAKHRSSVLAEVRQMLKKGEPCRLVATSLIEAGVDVDFPVVLRAEAGLDSIAQAAGRCNREGKRPLAESEVLVFAAANSDWAPPEELKQFAQAAREVMRLHPDDCLSMAAIERYFRILYWQKGAEELDAGNLLGLIERGRLDGLPYETLATKFRMIDSLQLPVIIPFDDEARAALRELEFADGCAAIARRLQPYLVQMPRKGYQALREAGAIQAAAGTRYGEQFMALVNPDLYHHQFGLHWDNPAFVSSERLCW

SEQ ID NO: 6 - protein, 224 aa, Artificial Sequence
  Description of Artificial Sequence Synthetic polypeptide
MAYGIRLMVWGERACFTRPEMKVERVSYDAITPSAARGILEAIHWKPAIRWVVDRIQVLKPIRFESIRRNEVGGKLSAVSVGKAMKAGRTNGLVNLVEEDRQQRATTLLRDVSYVIEAHFEMTDRAGADDTVGKHLDIFNRRARKGQCFHTPCLGVREFPASFRLLEEGSAEPEVDAFLRGERDLGWMLHDIDFADGMTPHFFRALMRDGLIEVPAFRAAEDKA

SEQ ID NO: 7 - protein, 587 aa, Artificial Sequence
  Description of Artificial Sequence Synthetic polypeptide
MILSALNDYYQRLLERGEANISPFGYSQEKISYALLLSAQGELLDVQDIRLLSGKKPQPRLMSVPQPEKRTSGIKSNVLWDKTSYVLGVSAKGGERTQQEHESFKTLHRQILVGEGDPGLQALLQFLDCWQPEQFKPPLFSEAMLDSNLVFRLDGQQRYLHETPAALALRTRLLADGDSREGLCLVCGQRQPLARLHPAVKGVNGAQSSGASIVSFNLDAFSSYGKSQGENAPVSEQAAFAYTTVLNHLLRRDEHNRQRLQIGDASVVFWAQADTPAQVAAAESTFWNLLEPPADDGQEAEKLRGVLDAVATGRPLHELDSLMEEGTRIFVLGLAPNTSRLSIRFWAVDSLAVFTQHLAEHFRDMHLEPLPWKTEPAIWRLLYATAPSRDGRAKTEDVLPQLAGEMTRAILTGSRYPRSLLANLIMRMRADGDVSGIRVALCKAVLAREARLSGKIHQEELPMSLDKDASNPGYRLGRLFAVLEGAQRAALGDRVNATIRDRYYGAASSTPATVFPILLRNTQNHLAKLRKEKPGLAVNLERDIGEIIDGMQSQFPRCLRLEDQGRFAIGYYQQAQARFNRGPDSVE

SEQ ID NO: 8 - protein, 289 aa, Artificial Sequence
  Description of Artificial Sequence Synthetic polypeptide
MTAISNRYEFVYLFDVSNGNPNGDPDAGNMPRLDPETNQGLVTDVCLKRKIRNYVSLEQESAPGYAIYMQEKSVLNNQHKQAYEALGIESEAKKLPKDEAKARELTSWMCKNFFDVRAFGAVMTTEINAGQVRGPIQLAFATSIDPVLPMEVSITRMAVTNEKDLEKERTMGRKHIVPYGLYRAHGFISAKLAERTGFSDDDLELLWRALANMFEHDRSAARGEMAARKLIVFKHEHAMGNAPAHVLFGSVKVERVEGDAVTPARGFQDYRVSIDAEALPQGVSVREYL

SEQ ID NO: 9 - DNA, 2667 bp, Artificial Sequence
  Description of Artificial Sequence Synthetic polynucleotide
atggaaccttttaaatatatatgccattactggggaaaatcctcaaaaagcttgacgaaaggaaatgatattcatctgttaatttatcattgccttgatgttgctgctgttgcagattgctggtgggatcaatcagtcgtactgcaaaatactttttgccgaaatgaaatgctatcaaaacagagggtgaaggcctggctgttatttttcattgctcttcatgatattggaaagtttgatatacgattccaatataaatcagcagaaagttggctgaaattaaatcctgcaacgccatcacttaatggtccatcaacacaaatgtgccgtaaatttaatcatggtgcagccggtctgtattggtttaaccaggattcactttcagagcaatctctcggggattttttcagtttttttgatgccgctcctcatccttatgagtcctggtttccatgggtagaggccgttacaggacatcatggttttatattacattcccaggatcaagataagtcgcgttgggaaatgccagcttctctggcatcttatgctgcgcaagataaacaggctcgtgaggagtggatatctgtactggaagcattatttttaacgccagcggggttatctataaacgatataccacctgattgttcatcactgttagcaggtttttgctcgcttgctgactggttaggctcctggactacaacgaatacctttctgtttaatgaggatgcgccttccgacataaatgctctgagaacgtatttccaggaccgacagcaggatgcgagccgggtattggagttgagtggacttgtatcaaataagcgatgttatgaaggtgttcatgcactactggacaatggctatcaacccagacaattacaggtgttagttgatgctcttccagtagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatatcggtattgccagttaaacaccgctttatccgtggtttgggaattggtcgaagtgttttaattgttgatgaagttcatgcttacgacacctatatgaacggcttgctggaggcagtgctcaaggctcaggctgatgtgggagggagtgttattcttctttccgcaaccctaccaatgaaacaaaaacagaaacttctggatacttatggtctgcatacagatccagtggaaaataactccgcatatccactcattaactggcgaggtgtgaatggtgcgcaacgttttgatctgctagctcatccagaacaactcccgccccgcttttcgattcagccagaacctatttgtttagctgacatgttacctgaccttacgatgttagagcgaatgatcgcagcggcaaacgcgggtgcacaggtctgtcttatttgcaatttggttgacgttgcacaagtatgctaccaacggctaaaggagctaaataacacgcaagtagatatagatttgtttcatgcgcgctttacgctgaacgatcgtcgtgaaaaagagaatcgagttattagcaatttcggcaaaaatgggaagcgaaatgttggacggatacttgtcgcaacccaggtcgtggaacaatcactcgacgttgattttgattggttaattactcagcattgtcctgcagatttgcttttccaacgattgggccgtttacatcgccatcatcgcaaatatcgtcccgctggttttgagattcctgttgccaccattttgctgcctgatggcgagggttacggacgacatgagcatatttatagcaacgttagagtcatgtggcggacgcagcaacatattgaggagcttaatggagcatccttatttttccctgatgcttaccggcaatggctggatagcatttacgatgatgcggaaatggatgagccagaatgggtcggcaatggcatggataaatttgaaagcgccgagtgtgaaaaaaggttcaaggctcgcaaggtcctgcagtgggctgaagaatatagcttgcaggataacgatgaaaccattcttgcggtaacgagggatggggaaatgagcctgccattattgccttatgtacaaacgtcttcaggtaaacaactgctcgatggccaggtctacgaggacctaagtcatgaacagcagtatgaggcgcttgcacttaatcgcgtcaatgtacccttcacctggaaacgtagtttttctgaagtagtagatgaagatgggttactttggctggaagggaaacagaatctggatggatgggtctggcagggtaacagtattgttattacctatacaggggatgaagggatgaccagagtcatccctgcaaatcccaaataa

SEQ ID NO: 10 - DNA, 1509 bp, Artificial Sequence
  Description of Artificial Sequence Synthetic polynucleotide
atgaatttgcttattgataactggatccctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctga

SEQ ID NO: 11 - DNA, 483 bp, Artificial Sequence
  Description of Artificial Sequence Synthetic polynucleotide
atggctgatgaaattgatgcaatggctttatatcgagcctggcaacaactggataatggatcatgtgcgcaaattagacgtgtttcagaacctgatgaattacgcgatatccctgcgttttataggctggtgcaaccttttggttgggaaaacccacgtcaccagcaggctcttttgcgcatggtgttttgcctgagcgcaggaaagaatgtcatccgacatcaggacaaaaaatcggagcaaacaacaggtatctcgttgggaagagctttagccaatagtggaagaattaacgagcgccgtatctttcaattaattcgggctgacagaacagccgatatggtccagttacgtcgattacttactcacgccgaacccgtacttgactggccattaatggccaggatgttgacctggtggggaaagcgcgaacgccagcaacttctggaagattttgtattgaccacaaacaaaaatgcgtaa

SEQ ID NO: 12 - DNA, 1092 bp, Artificial Sequence
  Description of Artificial Sequence Synthetic polynucleotide
atgtctaactttatcaatattcatgttctgatctctcacagcccttcatgtctgaaccgcgacgatatgaacatgcagaaagacgctattttcggcggcaaaagacgagtaagaatttcaagtcaaagccttaaacgtgcgatgcgtaaaagtggttattacgcacaaaatattggtgaatccagtctcagaaccattcatcttgcacaattacgtgatgttcttcggcaaaaacttggtgaacgttttgaccaaaaaatcatcgataagacattagcgctgctctccggtaaatcagttgatgaagccgaaaagatttctgccgatgcggttactccctgggttgtgggagaaatagcctggttctgtgagcaggttgcaaaagcagaggctgataatctggatgataaaaagctgctcaaagttcttaaggaagatattgccgccatacgtgtgaatttacagcagggtgttgatattgcgcttagtggaagaatggcaaccagcggcatgatgactgagttgggaaaagttgatggtgcaatgtccattgcgcatgcgatcactactcatcaggttgattctgatattgactggttcaccgctgtagatgatttacaggaacaaggttctgcacatctgggaactcaggaattttcatcgggtgttttttatcgttatgccaacattaacctcgctcaacttcaggaaaatttaggtggtgcctccagggagcaggctctggaaattgcaacccatgttgttcatatgctggcaacagaggtccctggagcaaaacagcgtacttatgccgcttttaaccctgcggatatggtaatggttaatttctccgatatgccactttctatggcaaatgcttttgaaaaagcggttaaagcgaaagatggctttttgcaaccgtctatacaggcgtttaatcaatattgggatcgcgttgccaatggatatggtctgaacggagctgctgcgcaattcagcttatctgatgtagacccaattactgctcaagttaaacaaatgcctactttagaacagttaaaatcctgggttcgtaataatggcgaggcgtga

SEQ ID NO: 13 - DNA, 675 bp, Artificial Sequence
  Description of Artificial Sequence Synthetic polynucleotide
atgagatcttatttgatcttgcggcttgctgggccaatgcaagcctgggggcagccgacctttgaaggaacgcgacctaccggaagatttccgacccgaagcgggttattagggctactcggggcttgtcttgggatccaacgtgatgatacttcttcattacaggcgttatcagagagtgtgcaatttgcagtgcgctgcgatgaactcattcttgacgatcgtcgtgtgtctgtaacggggttgcgtgattaccatacagtccttggagcgcgagaagattaccgtggtttgaaaagtcatgaaacgattcaaacatggcgcgaatatttatgtgatgcctcctttaccgtcgctctctggttaacaccccatgcaacgatggttatctcagaacttgaaaaagcagtattaaagcctcggtatacaccttacctggggcggagaagttgcccactaacacacccgctttttttggggacatgtcaggcatcggatcctcagaaggcgctattaaattatgagcccgttggcggcgatatatatagtgaggaatcagttacagggcatcatttaaaatttacggcgcgcgacgaaccgatgatcaccttgcctcgacaatttgcttcccgagaatggtatgtgattaaaggaggtatggatgtatctcagtaa

SEQ ID NO: 14 - DNA, 600 bp, Artificial Sequence
  Description of Artificial Sequence Synthetic polynucleotide
atgtatctcagtaaagtcatcattgccagggcctggagcagggatctttaccaacttcaccagggattatggcatttatttccaaacagaccggatgctgctcgtgattttctttttcatgttgagaagcgaaacacaccagaaggctgtcatgttttattgcagtcagcgcaaatgcctgtttcaactgccgttgcgacagtcattaaaactaaacaggttgaatttcaacttcaggttggtgttccactctattttcggcttcgggcaaatccgatcaaaactattctcgacaatcaaaagcgcctggacagtaaagggaatattaaacgctgtcgggttccgttaataaaagaagcagaacaaatcgcgtggttgcaacgtaaattgggcaatgcggcgcgcgttgaagatgtgcatcccatatcggaacggccacagtatttttctggtgatggtaaaagtggaaagatccaaacggtttgctttgaaggtgtgctcaccatcaacgacgcgccagcgttaatagatcttgtacagcaaggtattgggccagctaaatcgatgggatgtggcttgctatctttggctccactgtga

SEQ ID NO: 15 - protein, 888 aa, Artificial Sequence
  Description of Artificial Sequence Synthetic polypeptide
MEPFKYICHYWGKSSKSLTKGNDIHLLIYHCLDVAAVADCWWDQSVVLQNTFCRNEMLSKQRVKAWLLFFIALHDIGKFDIRFQYKSAESWLKLNPATPSLNGPSTQMCRKFNHGAAGLYWFNQDSLSEQSLGDFFSFFDAAPHPYESWFPWVEAVTGHHGFILHSQDQDKSRWEMPASLASYAAQDKQAREEWISVLEALFLTPAGLSINDIPPDCSSLLAGFCSLADWLGSWTTTNTFLFNEDAPSDINALRTYFQDRQQDASRVLELSGLVSNKRCYEGVHALLDNGYQPRQLQVLVDALPVAPGLTVIEAPTGSGKTETALAYAWKLIDQQIADSVIFALPTQATANAMLTRMEASASHLFSSPNLILAHGNSRFNHLFQSIKSRAITEQGQEEAWVQCCQWLSQSNKKVFLGQIGVCTIDQVLISVLPVKHRFIRGLGIGRSVLIVDEVHAYDTYMNGLLEAVLKAQADVGGSVILLSATLPMKQKQKLLDTYGLHTDPVENNSAYPLINWRGVNGAQRFDLLAHPEQLPPRFSIQPEPICLADMLPDLTMLERMIAAANAGAQVCLICNLVDVAQVCYQRLKELNNTQVDIDLFHARFTLNDRREKENRVISNFGKNGKRNVGRILVATQVVEQSLDVDFDWLITQHCPADLLFQRLGRLHRHHRKYRPAGFEIPVATILLPDGEGYGRHEHIYSNVRVMWRTQQHIEELNGASLFFPDAYRQWLDSIYDDAEMDEPEWVGNGMDKFESAECEKRFKARKVLQWAEEYSLQDNDETILAVTRDGEMSLPLLPYVQTSSGKQLLDGQVYEDLSHEQQYEALALNRVNVPFTWKRSFSEVVDEDGLLWLEGKQNLDGWVWQGNSIVITYTGDEGMTRVIPANPK

SEQ ID NO: 16 - protein, 502 aa, Artificial Sequence
  Description of Artificial Sequence Synthetic polypeptide
MNLLIDNWIPVRPRNGGKVQIINLQSLYCSRDQWRLSLPRDDMELAALALLVCIGQIIAPAKDDVEFRHRIMNPLTEDEFQQLIAPWIDMFYLNHAEHPFMQTKGVKANDVTPMEKLLAGVSGATNCAFVNQPGQGEALCGGCTAIALFNQANQAPGFGGGFKSGLRGGTPVTTFVRGIDLRSTVLLNVLTLPRLQKQFPNESHTENQPTWIKPIKSNESIPASSIGFVRGLFWQPAHIELCDPIGIGKCSCCGQESNLRYTGFLKEKFTFTVNGLWPHPHSPCLVTVKKGEVEEKFLAFTTSAPSWTQISRVVVDKIIQNENGNRVAAVVNQFRNIAPQSPLELIMGGYRNNQASILERRHDVLMFNQGWQQYGNVINEIVTVGLGYKTALRKALYTFAEGFKNKDFKGAGVSVHETAERHFYRQSELLIPDVLANVNFSQADEVIADLRDKLHQLCEMLFNQSVAPYAHHPKLISTLALARATLYKHLRELKPQGGPSNG

SEQ ID NO: 17 - protein, 160 aa, Artificial Sequence
  Description of Artificial Sequence Synthetic polypeptide
MADEIDAMALYRAWQQLDNGSCAQIRRVSEPDELRDIPAFYRLVQPFGWENPRHQQALLRMVFCLSAGKNVIRHQDKKSEQTTGISLGRALANSGRINERRIFQLIRADRTADMVQLRRLLTHAEPVLDWPLMARMLTWWGKRERQQLLEDFVLTTNKNA

SEQ ID NO: 18 - protein, 363 aa, Artificial Sequence
  Description of Artificial Sequence Synthetic polypeptide
MSNFINIHVLISHSPSCLNRDDMNMQKDAIFGGKRRVRISSQSLKRAMRKSGYYAQNIGESSLRTIHLAQLRDVLRQKLGERFDQKIIDKTLALLSGKSVDEAEKISADAVTPWVVGEIAWFCEQVAKAEADNLDDKKLLKVLKEDIAAIRVNLQQGVDIALSGRMATSGMMTELGKVDGAMSIAHAITTHQVDSDIDWFTAVDDLQEQGSAHLGTQEFSSGVFYRYANINLAQLQENLGGASREQALEIATHVVHMLATEVPGAKQRTYAAFNPADMVMVNFSDMPLSMANAFEKAVKAKDGFLQPSIQAFNQYWDRVANGYGLNGAAAQFSLSDVDPITAQVKQMPTLEQLKSWVRNNGEA

SEQ ID NO: 19 - protein, 224 aa, Artificial Sequence
  Description of Artificial Sequence Synthetic polypeptide
MRSYLILRLAGPMQAWGQPTFEGTRPTGRFPTRSGLLGLLGACLGIQRDDTSSLQALSESVQFAVRCDELILDDRRVSVTGLRDYHTVLGAREDYRGLKSHETIQTWREYLCDASFTVALWLTPHATMVISELEKAVLKPRYTPYLGRRSCPLTHPLFLGTCQASDPQKALLNYEPVGGDIYSEESVTGHHLKFTARDEPMITLPRQFASREWYVIKGGMDVSQ

SEQ ID NO: 20 - protein, 199 aa, Artificial Sequence
  Description of Artificial Sequence Synthetic polypeptide
MYLSKVIIARAWSRDLYQLHQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPL

SEQ ID NO: 21 - DNA, 3234 bp, Artificial Sequence
  Description of Artificial Sequence Synthetic polynucleotide
atgaatgtcctaattatctcacgctgtacgaaaaatgctcgcgtggaaagctgccgtattattgatcagtttgccgaacgcactggtgacgcagcatggcaaacggtgattactctggaaggcgttaatacactccgccgtttactgcgcaaaaccgccaggcgaaataccgcagtggcttgtcactggctgaagaagaatgggcaaacggagctgatgtggattgtcggcaatattcgtcgctttaatgctcaaggccgcgttcccacaaatcgaacaacgcaaatcagcctggtgaacaaagaagaaaatcgctggcagtgcgcggaaagtattgcactgctggcggctatcgctgggctatttcatgattttggtaaatcaggacgcagctttcagcaaagcctgactcaaaagaatacccgcagttatcaaccttaccgacatgaatggatctcactgcggctctttcaggcgtttgtcggtgatcggcccgacgatgtgtggctggcgaagcttggcgaactcacggcagatgatgaatctgcgattcttgagcgcctgcaaaaagacactcctcaattttcaaatagcccgttcagttcgctgcaaccattagccaaaacggtaggctggctgatcctttcccatcatcgaatgccggagaatctttctccacagtcggaacaatcatgtaaagggtgtgaaagttggctggagaagcaacttaatgctgactggaatgccgttaaccaccgcagagatgactggtcacaacaggattttgcgcacgtctggcagtttccatcaggaacgccgcttcaaagcgcgagctggcgggaaaaagcgcgtcagatagcccgccgggcaagcaacgcgctaggtttaaagacatatgggcagatggaacagttgttccccttacatatggcgcgtttttgtctgatgcttgccgatcattactattcctcaagcgatgcgcagcaaaaatggcaggacccggattatatcgtctgggcaaatagcgatcgccaaagcggcacgctcaaacaacgggtagacgaacaccttgtgggcgtcgcacatcatgcgtatctgcttgggcggcaactgatgcacacacgggcatcgcttccggcaattgcccgacataaaggttttagccagcgggccatagatgtgaattatcgctggcaagataaagcctgggatgtggcgcgagcgttacgtgaacgaagtcgtgaagagggatttttcggcgtcaatatggcctcgacggggtgtggtaaaacctttgcgaacgcccgcattatgtatgcacttgccgacgagcgagaagggtgccgctttaccgttgcgctgggattgcgcacgctgactttacaaaccggcgatgctctgcgcagtcggcttgggctgggtgaggatgagctggcggtactgattggttcggcggcagttaatcaactgtggcaacaggaaaaaatagacaatgattgcggaagtgcttcgcaagagtcgccggcagaggagcaacagttcgttaagtacgaaggcagcctgcataccgggacgctggaaaaatggttgaaggatgacagcaaacttaaggagttagtaagcgcgccgattctggtctcgacgattgatcatcttatttccgcgaccgagggcgtgcgaggcgggaaacagcttccggcgatgttgcgcctgttaaccagcgatttagtgctcgacgaacctgatgactttgatattgccgatcttcatgcggtatgtcgattgatgaactgggccgggatgctgggaacgcgggtattactttcttccgcgacgttaccgccaggtttgattcaggcgctgttcgcggcttatcttgctgggcgaaaaatgtggcaggcgtcttgtggtatcaacggcaggcccgtcaatatatgctgcgcctggtttgatgaaaaagatgctgatgccacgcaaatttacgacggacctgggtttcgtgatgctcatgccaaatttgttgcacgacgagcggtaatgctggctgaaaaagaacgattgcatttcgggcgtgtagcttcggtttcatcagcatcaggcgcgatacaagatgttaccgaaagtgtggcccagacggtgcatacgcaaatgctgaagttgcatcaggcgcatcgccagcgacacgaaagcggtaaaaccgtttcgctgggcctggtgcgcttcgcgaatatcaatccactggttgcggtgaccaaagccctgattgtcattccttcaccagaagatgtctgcattcattactgtgtttaccactcccgacacccgctggcggtgcgttcagatattgagaagcgactggatcgcgcttttactcgtcataacgaactcgatttctggaacaacgaagatatcgcagatgccttacataaccgtccggaatcgcaccatctgtttgtggtgttggggacatcggttatagaagtggggcgtgactgggatgcggactggggaattatcgaaccaagctccatgcgttcgataattcaatttgccggacgtattcagcgccaccgtagaaatgtgccgaccagtgagaatctggttattttgcgtagcaatatcaaatcattgcagaaaaaagcccccgcattttgtaagcctggtttcgaaacaaaagaacacttgctgaatagccacgatatttgcgaactgattccgatcgcggactaccggacgattaacgccttgccgcggatcctgtctgccaataatgccaacaaactggcggagctagaacatagtcgtttatccgccgaattgatgagcagtaaaagcaaacatgtggtggcggcgcaatggtggcgtgcaccgctaagctggaacggctggatgcagcgccagacgccatttcgttattcccctttgccggaggcggtgttttttctgcatatggatgatgaagaaagcgaagcgcgtttctattcccgcacgggcgacaacgagcgtaaagcgcaagggaatttcaggcgagaagaggtgagatgcgccagtggcgtcgtttgctggggagtgatggattaccaacacgttttgttggatttggctgaaaagcagagttgtgagatagcaagcgttggggaaaagtttgcggaagtgagagtacccgtcagtgaagaagatgcggtcgatgactggcgttatcacccctggttaggtgtgtttcgcaacatgtga

SEQ ID NO: 22 - DNA, 1308 bp, Artificial Sequence
  Description of Artificial Sequence Synthetic polynucleotide
atggaaaaaacggcgctaatacaatttattactgactacatcgcttcgcgaaggcaacacaaaatcgatgcctttgaaaaagaagccgcaaagcgagtggaacagggcgaggacgccagcgttatcgctcaggagcggcaggaactcgaagcacgctacttgccacgtaactggttaacggatgcggcaaagcgtgccgggcaaatcaaattagtcactcatggcgctaaattcagtcatggtgattctaaagccagtagcttttatctggaaacctccgctaacgaaagctatttaaacacagcatcgttggcgaatgtcgctactgacgcgattggtaacgccgctgcacttgacgtagcaaagttgctacaaaccgatgtgaacggagattcactgcttgccagcctgaagcgtggtgattatcaggcattaagtacgtttgcagaggataaagtgcaactggagctgtgggtagctgggtttagccaggcatggactactggacggccttccagccataaactggcgaaacaaatctatttccctgttgcggatggttatcacctgttatgcccactgttttccagttcactggcccaggcgatgtatgaaaagctaaccgccgggcgctttagtgaggaatcgaaagccattcgcgatgcccgcaaagcaggaaaatggcactcccagccagatatccggtttcctaatcttgccgaaatgcatttcggcggcacaaaaccgcagaatatttctttgctaaacagtgtgcgtggtgggcgcgtctggctgttgccgtcaatgccgcctgcctgggggccgctggatcgcgcaccacaaaaaatgcacagtatttttgccttacgcggcgatttcaatagggcggcttcaggcatcatagcccggatgacttacctgctgaaggttgacacaaataatgttcatatccgaactgtgcgggcgaaatatattgatgagttgatcgatttgctgttcatgcaggcatccgcttttcagcaagaaaaatggcagggatggtccgcacaatcaccagacttacctcgccaccaacaactttggctggacccgtggcgaagcctctcagatgagactttcaaacaggagcgtgagaaaaacgactggcaagtaactgtcgccgatgatttcgcacgctggctgaactatcgcctgaaaaaatccagttttgacgtaggcgctgtcgaacaaaaagagtggcgcagccagtctctgttcgctcaacggatgcgtgaaatggaagctgttttgcaggaggcgctgaaatga

SEQ ID NO: 23 - DNA, 924 bp, Artificial Sequence
  Description of Artificial Sequence Synthetic polynucleotide
atgagttatctgctcttgttaccccatatacgcatcgaaaacgctaacgctgtctccgggttgacctggggattcccgtcgatgacccattttctcggttatgtccatgcgctttctcgcaaagtcgtggatgaatttggcgtaagttttgatggttgtgcggtagtaagccatgaacagcatattcaggcgtacagctctgggcgcgattttcagttcgctctcacccgtaacccattaaccagagaaggtaaaaccgcctcgttcaatgaagagggccgcatgcacttgacggtttctctactgattgaatgtaatggcgaaatcaccaatggtgaatatggcagaaaagttctttgtgactatctgaaaatgctttgccagagccacaaactggcgggcggcagcattgtcagtatgcgcgatccgcagctttttcatgcgcctgaagatgaaaagcaactgcgtaaaatcgtctggcggttaatgccgggttatgcgctttacgatcgcagtgaatggctggctgaacatcaccaacaacatcccgacatctcgttgctggatgcctggcttgatttcgccgcgattaaatatcaggccgaatctccggctgaagacaacagcgcaaaatgggtataccagccgaagccaatgcctggtttcctggtaccgttaatgtgtggctaccaacgcatttctcctgtttacgcacccggtgaagttgaaaatgcgcgcgacaccgtgacgccatttgcctttgccgaagcggtatatggcattggcgaatggcgcggattacatcgcactacagacttgcaagcgctgatgtggcgttaccacacaacggacactggctactattgttcggcaacgccagttgttgatgattttacctttaatgaatatgacgatttagaataa

SEQ ID NO: 24 - DNA, 1002 bp, Artificial Sequence
  Description of Artificial Sequence Synthetic polynucleotide
atggcaaaggctcctgtagcaataaaaacggcgtcggtactggcatttgagcgcaaactggcaacctctgacgcggtaatgtacgcaggtaactggcaaggaaatgaatggcaacccattgagattcaggagaaagcggtccgcggaaccatctctaatcgcctgaaaaatgccattgtcagcgatcccacaaaactggatgctgaaatccaaaaagccaacctgcaacgggtggatgtggcttcgcttccggtagatacggatacgttaaaggtggtgtttacgctgcgtgtgctgggtaatttatccacaccgtcagtatgtaatgatatggcttaccaggaggcgctttccggggtcattgaaggttacatcagcgaacatggctttaaagagctggcgcttcgctatgcgctgaatttggcaaatggtcgttttctgtggcgcaaccgcattggtgccgaacagatccaggtgaaagtgactgctaacgagacttcatggacttttaacagccatgacttctctttacgtcaatttgaccagggacaggcagcggtcacgcagctggcggccattatagagcaaggattaagcgcaaaagagtgggtgatgcttaccgttgaagcgcaggttcgtcttggtgccgggcaagaagtttttccgtcgcaggagctggtactcgacagcaacagtagtaaaagtcgcgtgttataccaggtggcaggcattgccggtattcactctcagaagattggcaatgctttacgtaccattgatacctggcatccaaaggttgatgaattgggggctatagccgtggaaccttacggttctgtaaccagtcgcggtgtggcctgccgtcaaccgaaagaaaaactcgacttttataccttactggacaattgggtgactaaagggatgaaaccagacgttaagcagcagcactacgtgatggctgtgctgatccgtggcggtgtctttggtgagaaatcggaataa

SEQ ID NO: 25 - DNA, 555 bp, Artificial Sequence
  Description of Artificial Sequence Synthetic polynucleotide
atggatcactaccttgagatccgcgttctgccagatcctgaattttcgagcgaaatgttgatggcggcgttatttgcaaaactgcatcgggtattaggtgcaagagggcaaggcgatatcggcgtgagtttcccggacgtaaatgttatgcctggaacgcatttacgcttgcatggtagcgcccaggcattgcaagaacttgaagcctcgacgtggcgtaaagggctgacagattactgccaatgttcgcctgtcacgccagttcctgaaattaaaggctggcgagtcgtaagtcgtgtacaggtaaaaagtaaccctcagcgtctgttaaggcgttcagtgaaaaaaggctggctaactgaagaacaggcaattgaacgactggcgacgcaggctgaacagcggaccgatttaccctttcttaatatgaagagtttgtcgagtcagcaacagttcaagttgtttatccgtcatggtgatttgctcaaagaaccggttaaaggggaatttagtagttatggattaagcgcaaccgccactattccctggttttga

SEQ ID NO: 26 - protein, 1077 aa, Artificial Sequence
  Description of Artificial Sequence Synthetic polypeptide
MNVLIISRCTKNARVESCRIIDQFAERTGDAAWQTVITLEGVNTLRRLLRKTARRNTAVACHWLKKNGQTELMWIVGNIRRFNAQGRVPTNRTTQISLVNKEENRWQCAESIALLAAIAGLFHDFGKSGRSFQQSLTQKNTRSYQPYRHEWISLRLFQAFVGDRPDDVWLAKLGELTADDESAILERLQKDTPQFSNSPFSSLQPLAKTVGWLILSHHRMPENLSPQSEQSCKGCESWLEKQLNADWNAVNHRRDDWSQQDFAHVWQFPSGTPLQSASWREKARQIARRASNALGLKTYGQMEQLFPLHMARFCLMLADHYYSSSDAQQKWQDPDYIVWANSDRQSGTLKQRVDEHLVGVAHHAYLLGRQLMHTRASLPAIARHKGFSQRAIDVNYRWQDKAWDVARALRERSREEGFFGVNMASTGCGKTFANARIMYALADEREGCRFTVALGLRTLTLQTGDALRSRLGLGEDELAVLIGSAAVNQLWQQEKIDNDCGSASQESPAEEQQFVKYEGSLHTGTLEKWLKDDSKLKELVSAPILVSTIDHLISATEGVRGGKQLPAMLRLLTSDLVLDEPDDFDIADLHAVCRLMNWAGMLGTRVLLSSATLPPGLIQALFAAYLAGRKMWQASCGINGRPVNICCAWFDEKDADATQIYDGPGFRDAHAKFVARRAVMLAEKERLHFGRVASVSSASGAIQDVTESVAQTVHTQMLKLHQAHRQRHESGKTVSLGLVRFANINPLVAVTKALIVIPSPEDVCIHYCVYHSRHPLAVRSDIEKRLDRAFTRHNELDFWNNEDIADALHNRPESHHLFVVLGTSVIEVGRDWDADWGIIEPSSMRSIIQFAGRIQRHRRNVPTSENLVILRSNIKSLQKKAPAFCKPGFETKEHLLNSHDICELIPIADYRTINALPRILSANNANKLAELEHSRLSAELMSSKSKHVVAAQWWRAPLSWNGWMQRQTPFRYSPLPEAVFFLHMDDEESEARFYSRTGDNERKAQGNFRREEVRCASGVVCWGVMDYQHVLLDLAEKQSCEIASVGEKFAEVRVPVSEEDAVDDWRYHPWLGVFRNM

SEQ ID NO: 27 - protein, 435 aa, Artificial Sequence
  Description of Artificial Sequence Synthetic polypeptide
MEKTALIQFITDYIASRRQHKIDAFEKEAAKRVEQGEDASVIAQERQELEARYLPRNWLTDAAKRAGQIKLVTHGAKFSHGDSKASSFYLETSANESYLNTASLANVATDAIGNAAALDVAKLLQTDVNGDSLLASLKRGDYQALSTFAEDKVQLELWVAGFSQAWTTGRPSSHKLAKQIYFPVADGYHLLCPLFSSSLAQAMYEKLTAGRFSEESKAIRDARKAGKWHSQPDIRFPNLAEMHFGGTKPQNISLLNSVRGGRVWLLPSMPPAWGPLDRAPQKMHSIFALRGDFNRAASGIIARMTYLLKVDTNNVHIRTVRAKYIDELIDLLFMQASAFQQEKWQGWSAQSPDLPRHQQLWLDPWRSLSDETFKQEREKNDWQVTVADDFARWLNYRLKKSSFDVGAVEQKEWRSQSLFAQRMREMEAVLQEALK

SEQ ID NO: 28 - protein, 307 aa, Artificial Sequence
  Description of Artificial Sequence Synthetic polypeptide
MSYLLLLPHIRIENANAVSGLTWGFPSMTHFLGYVHALSRKVVDEFGVSFDGCAVVSHEQHIQAYSSGRDFQFALTRNPLTREGKTASFNEEGRMHLTVSLLIECNGEITNGEYGRKVLCDYLKMLCQSHKLAGGSIVSMRDPQLFHAPEDEKQLRKIVWRLMPGYALYDRSEWLAEHHQQHPDISLLDAWLDFAAIKYQAESPAEDNSAKWVYQPKPMPGFLVPLMCGYQRISPVYAPGEVENARDTVTPFAFAEAVYGIGEWRGLHRTTDLQALMWRYHTTDTGYYCSATPVVDDFTFNEYDDLE

SEQ ID NO: 29 - protein, 333 aa, Artificial Sequence
  Description of Artificial Sequence Synthetic polypeptide
MAKAPVAIKTASVLAFERKLATSDAVMYAGNWQGNEWQPIEIQEKAVRGTISNRLKNAIVSDPTKLDAEIQKANLQRVDVASLPVDTDTLKVVFTLRVLGNLSTPSVCNDMAYQEALSGVIEGYISEHGFKELALRYALNLANGRFLWRNRIGAEQIQVKVTANETSWTFNSHDFSLRQFDQGQAAVTQLAAIIEQGLSAKEWVMLTVEAQVRLGAGQEVFPSQELVLDSNSSKSRVLYQVAGIAGIHSQKIGNALRTIDTWHPKVDELGAIAVEPYGSVTSRGVACRQPKEKLDFYTLLDNWVTKGMKPDVKQQHYVMAVLIRGGVFGEKSE

SEQ ID NO: 30 - protein, 184 aa, Artificial Sequence
  Description of Artificial Sequence Synthetic polypeptide
MDHYLEIRVLPDPEFSSEMLMAALFAKLHRVLGARGQGDIGVSFPDVNVMPGTHLRLHGSAQALQELEASTWRKGLTDYCQCSPVTPVPEIKGWRVVSRVQVKSNPQRLLRRSVKKGWLTEEQAIERLATQAEQRTDLPFLNMKSLSSQQQFKLFIRHGDLLKEPVKGEFSSYGLSATATIPWF

SEQ ID NO: 31 - DNA, 97 bp, Artificial Sequence
  Description of Artificial Sequence Synthetic oligonucleotide
acaagcggcacattgtgcctattgcgaattaggcacaatgtgcctaatctaacgtcatgccagccacaacggcgaggcgccaagaaggatagaagcc

SEQ ID NO: 32 - DNA, 34 bp, Artificial Sequence
  Description of Artificial Sequence Synthetic oligonucleotide
agatagtccgggatatcggccggatgcttaacgt

SEQ ID NO: 33 - DNA, 34 bp, Artificial Sequence
  Description of Artificial Sequence Synthetic oligonucleotide
ccgagcgatggtccggttatgcagaaaaagacga

SEQ ID NO: 34 - DNA, 32 bp, Artificial Sequence
  Description of Artificial Sequence Synthetic oligonucleotide
gtcgcgccccgcacgggcgcgtggattgaaac

SEQ ID NO: 35 - DNA, 261 bp, Artificial Sequence
  Description of Artificial Sequence Synthetic polynucleotide
acaagcggcacattgtgcctattgcgaattaggcacaatgtgcctaatctaacgtcatgccagccacaacggcgaggcgccaagaaggatagaagccgtcgcgccccgcacgggcgcgtggattgaaacagatagtccgggatatcggccggatgcttaacgtgtcgcgccccgcacgggcgcgtggattgaaacccgagcgatggtccggttatgcagaaaaagacgagtcgcgccccgcacgggcgcgtggattgaaac

SEQ ID NO: 36 - DNA, 761 bp, Artificial Sequence
  Description of Artificial Sequence Synthetic polynucleotide
tttacagctagctcagtcctagggactgtgctagcattaaagaggagaaaatggtttcaaaaggcgaagaagacaacatggcgattatcaaggaatttatgcgtttcaaggtccacatggaaggcagcgtcaatggtcacgaatttgaaattgaaggcgaaggtgaaggccgtccgtatgaaggcacccagacggcaaaactgaaggtcaccaaaggcggtccgctgccgtttgcttgggatattctgtcaccgcaattcatgtatggttcgaaagcgtacgttaagcatccggccgatatcccggactatctgaaactgtcctttccggaaggcttcaaatgggaacgtgttatgaacttcgaagatggcggtgtggttaccgtcacgcaggatagctctctgcaagacggtgaatttatttataaagtgaagctgcgcggcaccaatttcccgagcgatggtccggttatgcagaaaaagacgatgggctgggaagcgagttccgaacgtatgtacccggaagacggtgccctgaaaggcgaaatcaagcagcgcctgaaactgaaggatggcggtcactatgacgcagaagtgaaaaccacgtacaaggctaaaaagccggtccaactgccgggtgcatacaacgtgaacatcaagctggatatcaccagccataacgaagactatacgatcgttgaacagtacgaacgtgcagaaggccgccactctaccggcggtatggatgaactgtacaaataa

SEQ ID NO: 37 - DNA, 31 bp, Escherichia coli
atattacggattgtcgcataggcgaaattat

SEQ ID NO: 38 - DNA, 34 bp, Escherichia coli
tcctccatccatttttttttaatacgtggaatgt

SEQ ID NO: 39 - DNA, 32 bp, Escherichia coli
tttctgaattactctaatcaaaaaacgtgcat

SEQ ID NO: 40 - DNA, 31 bp, Escherichia coli
tatatcattttcacttccggctccaccggta

SEQ ID NO: 41 - DNA, 34 bp, Escherichia coli
gcgcgctggatggtctgggcattgcactgacggc

SEQ ID NO: 42 - DNA, 32 bp, Escherichia coli
ctggacacgcctgctgcgccgttgcctgaagt

SEQ ID NO: 43 - DNA, 31 bp, Bacteroides fragilis
agttatgcatcgtgcatatggtgcgcaacag

SEQ ID NO: 44 - DNA, 34 bp, Bacteroides fragilis
ctctagctgtatcctggtgctgaatcactttccc

SEQ ID NO: 45 - DNA, 32 bp, Bacteroides fragilis
ttcgattaatagtgagtttaaaggaaatatga

SEQ ID NO: 46 - DNA, 31 bp, Enterococcus faecium
ggtgataaagcacgaacccataaagaggata

SEQ ID NO: 47 - DNA, 34 bp, Enterococcus faecium
gtgctttatcaccggtatagaacatatcaaaaag

SEQ ID NO: 48 - DNA, 32 bp, Enterococcus faecium
ggtttgttgatgaagaagaagtggttgttcaa

(19) United States
(12) Patent Application Publication
(10) Pub. No.: US 2024/0067935 A1
(43) Pub. Date: Feb. 29, 2024

(54) ALTERING THE NORMAL BALANCE OF MICROBIAL POPULATIONS

(71) Applicant: Locus Biosciences, Inc., Morrisville, NC (US)

(72) Inventors: David G. OUSTEROUT, Raleigh, NC (US); Kurt SELLE, Raleigh, NC (US); Cameron PRYBOL, 523 Davis Drive, NC (US)

(21) Appl. No.: 18/269,081

(22) PCT Filed: Dec. 22, 2021
(86) PCT No.: PCT/US2021/064995

§ 371 Date: Jun. 22, 2023

Related U.S. Application Data
(60) Provisional application No. 63/130,229, filed on Dec. 23, 2020.

(51) Int. Cl. C12N 7/00; A61P 31/04; C12N 9/22

(52) U.S. Cl. CPC C12N 7/00; A61P 31/04; C12N 9/22; C12N 2310/20; C12N 2795/00021

(57) ABSTRACT

Disclosed herein are compositions comprising exogenous antimicrobial agents and methods of use.

### CROSS REFERENCE

This application claims the benefit of U.S. Provisional Application No. 63/130,229, filed on Dec. 23, 2020, which is incorporated herein by reference in its entirety.

### SUMMARY

Disclosed herein, in certain embodiments, are phage compositions comprising exogenous microbial systems and methods of use thereof.

In one aspect, provided herein is a recombinant bacteriophage comprising an exogenous antimicrobial agent specific for a first microbe, wherein (a) the first microbe comprises a first microbe of Table 2, column 1, and/or (b) the first microbe comprises a target DNA sequence at least 10 nucleobasesin length and (i) at least 80% identical to a target DNA of Table 2, column 3 and/or 4, and/or (ii) at least 80% identical to any one of SEQ ID NOS: 37-48.

In some embodiments, the first microbe comprises the first microbe of Table 2, column 1.

In some embodiments, the first microbe comprises the target DNA sequence at least 10 nucleobasesin length and at least 80% identical to a target DNA of Table 2, column 3 and/or 4.

In some embodiments, the first microbe comprises the target DNA sequence at least 10 nucleobasesin length and at least 80% identical to a target DNA of Table 3.

In some embodiments, the antimicrobial agent comprises one or more components of a CRISPR system. In some embodiments, CRISPR system and the antimicrobial agent comprise a CRISPR array comprising a spacer sequence at least 80% identical to any one of SEQ ID NOS: 37-48. In some embodiments, the CRISPR system and the antimicrobial agent comprise a CRISPR array comprising a spacer sequence at least 80% identical to at least 10 contiguous nucleobases of the target DNA of Table 2, column 3 and/or 4. In some embodiments, the CRISPR system and the antimicrobial agent comprise a nucleic acid encoding a CRISPR nuclease. In some embodiments, the CRISPR nuclease comprises Cas3, Cas3′ and Cas3″, Cpf1, or Cas9. In some embodiments, the CRISPR system comprises a Type I CRISPR-Cas system, a Type II CRISPR-Cas system, or a Type V CRISPR-Cas system. In some embodiments, the CRISPR system comprises the Type I CRISPR-Cas system. In some embodiments, the Type I CRISPR-Cas system comprises Cas3. In some embodiments, the Type I CRISPR-Cas system is a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system. In some embodiments, the Type I CRISPR-Cas system is an E. coli Type I-F system (e.g., ECIF). In some embodiments, the Type I CRISPR-Cas system is an E. coli Type I-E system (e.g., ECIE). In some embodiments, the Type I CRISPR-Cas system is a P. aeruginosa Type 1-C system (e.g., PAIC).

The recombinant bacteriophage of any one of the embodiments above, prepared by a method comprising introducing into a first bacteriophage the exogeneous antimicrobial agent. In some embodiments, the first bacteriophage is a P1 phage, a M13 phage, a λ phage, a T4 phage, a T7 phage, a T7m phage, a φC2 phage, a φCD27 phage, a φNM1 phage, a Bc431 v3 phage, a φ10 phage, a φ25 phage, a φ151 phage, a A511-like phage, a B054, a 0176-like phage, a Campylobacterphage, p004k (PTA-127149), p00c0 (PTA-127143), p00ex (PTA-127145), p00jc (PTA-127147), p00ke (PTA-127148), p5516 (PTA-127151), p0046-9, p0033s-6, p0071-16, p0033L-10, p00ex-2, p0031-8, p004k-5, p0045-9, p0078-4, p00dd-1, p00E8-3, p00Jc-2, p006008, p006009, p006010, p006012, p006013, p006016, p006018, p006071, p006072, p006098, p006099, p006128, p006129, p5852, p5853, p3854-40-8, p3855-56-3, p4075, p4076, p4077, p4078, p4079, p4082, p4083, p4084, p4085, p4087, p4088, p4090, p4092, p4093, p4094, p5097, p5496, p5497, p5499, p5501, p5503, p5505, p5506, p5507, p5508, p5509, p5511, p5512, or any unmodified bacteriophage. In some embodiments, the recombinant bacteriophage is an obligately lytic bacteriophage. In some embodiments, the recombinant bacteriophage targets the first or the first microbe and a second microbe, wherein the first microbe is a microbe genus, or a microbe species or a microbe sub-species. In some embodiments, the microbe species is E. coli, B. fragilis or Enterococcis sp. In some embodiments, the exogenous antimicrobial agent is not specific for a second microbe. In some embodiments, the first microbe comprises the target DNA sequence and the second microbe does not comprise the target DNA sequence. In some embodiments, the exogeneous antimicrobial agent selectively kills the first microbe and not the second microbe. In some embodiments, the recombinant bacteriophage is capable of infecting the first microbe and the second microbe. In some embodiments, the second microbe is a second microbe of Table 2, column 6.

In some embodiments, the first microbe comprises an adherent-invasive E. coli and the second microbe comprises a commensal E. coli. In some embodiments, the first microbe comprises a pks+E. coli and the second microbe comprises a commensal E. coli. In some embodiments, the first microbe comprises enterotoxigenic B. fragilis and the second microbe comprises a commensal B. fragilis. In some embodiments, the first microbe comprises a vancomycin resistant Enterococcus (VRE) and the second microbe comprises a vancomycin sensitive Enterococcus. In some embodiments, (a) the first and the second microbe are targeted by the bacteriophage, and (b) the exogenous antimicrobial agent preferentially targets the first microbe over the second microbe.

In one aspect, provided herein is a method of microbial killing or restricting the expansion of a microbial population, the method comprising combining the recombinant bacteriophage of any one of the embodiments described above with the first microbe of any one of the embodiments described above.

In one aspect, provided herein is a method of selective microbial killing or selectively restricting the expansion of a microbial population, the method comprising combining the recombinant bacteriophage of any one of the embodiments described above with the first and second microbe of any one of the embodiments described above, wherein the first microbe is killed by the recombinant bacteriophage at a higher efficiency relative to the second microbe; and/or wherein expansion of the first microbe is restricted by the bacteriophage relative to expansion of the second microbe. In some embodiments, the first microbe is killed by the recombinant bacteriophage and the second microbe is not killed by the recombinant bacteriophage; and/or wherein the expansion of the first microbe is restricted by the recombinant bacteriophage and the expansion of the second microbe is not restricted by the recombinant bacteriophage.

In one aspect, provided herein is a method of treating or preventing a disease or condition in a subject in need thereof, the method comprising administering to the subject the recombinant bacteriophage of an embodiment above, wherein disease or condition is inflammatory bowel disease or colorectal cancer. In some embodiments, the first microbe comprises an adherent-invasive E. coli and the second microbe comprises a commensal E. col.

In one aspect, provided herein is a method of treating or preventing a disease or condition in a subject in need thereof, the method comprising administering to the subject the recombinant bacteriophage of an embodiment above, wherein disease or condition is inflammatory bowel disease. In some embodiments, the first microbe comprises a pks+E. coli and the second microbe comprises a commensal E. coli.

In one aspect, provided herein is a method of treating or preventing a disease or condition in a subject in need thereof, the method comprising administering to the subject the recombinant bacteriophage of an embodiment above, wherein disease or condition is colon cancer. In some embodiments, the first microbe comprises a pks+E. coli and the second microbe comprises a commensal E. coli.

In one aspect, provided herein is a method of treating or preventing a disease or condition in a subject in need thereof, the method comprising administering to the subject the recombinant bacteriophage of an embodiment above, wherein disease or condition is inflammatory bowel disease. In some embodiments, the first microbe comprises enterotoxigenic B. fragilis and the second microbe comprises a commensal B. fragilis.

In one aspect, provided herein is a method of treating or preventing a disease or condition in a subject in need thereof, the method comprising administering to the subject the recombinant bacteriophage of an embodiment above, wherein disease or condition is colon cancer. In some embodiments, the first microbe comprises enterotoxigenic B. fragilis and the second microbe comprises a commensal B. fragilis.

In one aspect, provided herein is a method of treating or preventing a disease or condition in a subject in need thereof, the method comprising administering to the subject the recombinant bacteriophage of an embodiment above, wherein disease or condition is an opportunistic bacterial infection caused by vancomycin-resistant enterococci (VRE) in patients undergoing chemotherapy or surgery or in patients at high risk of developing a VRE infection. In some embodiments, the first microbe comprises a vancomycin resistant Enterococcus (VRE) and the second microbe comprises a vancomycin sensitive Enterococcus.

In certain aspects, disclosed herein is a recombinant bacteriophage capable of infecting a first microbial population and a second microbial population, the recombinant bacteriophage comprising a nucleic acid sequence encoding an exogenous antimicrobial agent that targets and kills the first microbial population, wherein the recombinant bacteriophage kills the first microbial population to a greater degree as compared to the second microbial population. In some embodiments, the recombinant bacteriophage is P1 phage, a M13 phage, a λ phage, a T4 phage, a T7 phage, a T7m phage, a φC2 phage, a φCD27 phage, a φNM1 phage, Bc431 v3 phage, φ10 phage, φ25 phage, φ151 phage, A511-like phages, B054, 0176-like phages, or Campylobacter phages (such as NCTC 12676 and NCTC 12677). In some embodiments, the recombinant bacteriophage is a recombinant lytic bacteriophage. In some embodiments, the recombinant bacteriophage comprises a second nucleic acid sequence encoding an operable lytic gene capable of inducing lysis of the first microbial population or the second microbial population during a lytic cycle of the recombinant bacteriophage. In some embodiments, the recombinant bacteriophage is rendered lytic by the removal, replacement, or inactivation of at least one lysogenic gene. In some embodiments, the lysogenic gene is selected from the group consisting of cI repressor, cII, lexA, and int. In some embodiments, the recombinant bacteriophage is replication competent. In some embodiments, the exogenous antimicrobial agent comprises an endonuclease or an exonuclease or an antibiotic peptide or a biologically active fragment thereof. In some embodiments, the exogenous antimicrobial agent is an endonuclease. In some embodiments, the endonuclease is a Cas polypeptide or a biologically active fragment thereof. In some embodiments, the Cas polypeptide is selected from the group consisting of: a Type I Cas polypeptide, a Type II Cas polypeptide, a Type III Cas polypeptide, a Type IV Cas polypeptide, a Type V Cas polypeptide, and a Type VI Cas polypeptide. In some embodiments, the Cas polypeptide is a Type I Cas polypeptide. In some embodiments, the Type I polypeptide is Cas3. In some embodiments, the recombinant bacteriophage further comprises a third nucleic acid sequence. In some embodiments, the third nucleic acid sequence comprises a spacer sequence or a crRNA that is complementary to a target nucleotide sequence in the first microbial population.

In certain aspects, disclosed herein is a method comprising contacting a population of the first microbial population and the second microbial population with the recombinant bacteriophage disclosed herein. In some embodiments, the second microbial population replicates or survives at an increased rate compared to the first microbial population. In some embodiments, the first microbial population and the second microbial population are different strains of bacteria. In some embodiments, the genome of the first microbial population and the second microbial population are at least about 85% identical, 90% identical, 95% identical, 97.5% identical, or 99% identical. In some embodiments, the first microbial population comprises a bacterial species selected from the group consisting of E. coli, Bacteriodes spp (e.g., Bacteroides fragilis, Bacteroides gingivalis, Bacteroides melaninogenicus Bacteroides thetaiotamicron), Enterococcus spp (e.g., Enterococcus avium, Enterococcus durans, Enterococcus faecalis, Enterococcus faecium, Enterococcus gallinarum, Enterococcus maloratus), Shigella, Hafnia alvei, Pseudomonas aeruginosa, Morganella morganii, Pseudomonas putida, Citrobacter koseri, Klebsiella pneumonia, Campylobacter jejuni, and Mycobacterium avium subspecies paratuberculosis. In some embodiments, the second microbial population comprises a bacterial species selected from the group consisting of E. coli, Bacteroides spp, Enterococcus spp, Shigella, Hafnia alvei, Pseudomonas aeruginosa, Morganella morganii, Pseudomonas putida, Citrobacter koseri, Klebsiella pneumonia, and Campylobacter jejuni. In some embodiments, the first microbial population comprises an adherent-invasive E. coli strain and the second microbial population comprises a commensal E. coli strain. In some embodiments, the first microbial population comprises an E. coli strain comprising pks+sequences and the second microbial population comprises a commensal E. coli strain. In some embodiments, the first microbial population comprises a enterotoxigenic B. fragilis strain and the second microbial population comprises a commensal B. fragilis strain. In some embodiments, the first microbial population is selected from the group consisting of a shiga toxin-producing E. coli (STEC) (e.g., E. coli 0157), verocytotoxin-producing E. coli (VTEC), or enterohemorrhagic E. coli (EHEC) strains, and the second microbial population comprises a commensal E. coli strain. In some embodiments, the first microbial population comprises a Shigella spp. and the second microbial population comprises a commensal E. coli strain. In some embodiments, the first microbial population comprises a Enterococcus spp. and the second microbial population comprises a different Enterococcus spp. In some embodiments, the first Enterococcus spp is E. faecalis or E. faecium. In some embodiments, the E. faecalis or E. faecium is vancomycin-resistant. In some embodiments, the first microbial population comprises a enteropathogenic E. coli (EPEC) strain and the second microbial population comprises a commensal E. coli strain. In some embodiments, the first microbial population comprises a first Bacteroides spp. and the second microbial population comprises a second Bacteriodies spp. In some embodiments, the first Bacteroides spp. is selected from the group consisting of B. fragilis and B. thetaiotomicron. In some embodiments, the first microbial population comprises a species of Cutibacterium acnes that leads to acne and skin inflammation and the second microbial population comprises a species of C. acnes that promotes healthy skin. In some embodiments, the first microbial population comprises a microbial species found in a gut microbiome of a formula-fed infant and the second microbial population comprises a microbial species found in a gut microbiome of a breast-fed infant. In some embodiments, the first microbial population comprises a first species of Campylobacter jejuni associated with anxiety and the second microbial population comprises a second species of Campylobacter jejuni. In some embodiments, the first microbial population causes elevated levels of IgM and IgA relative to the second microbial population in a subject. In some embodiments, the first microbial population comprises a target sequence that is absent in the second microbial population. In some embodiments, the target sequence comprises a sequence selected from the group consisting of an AIEC specific DNA sequence, pks+DNA sequence, a sequence encoding an enterotoxin, a sequence encoding a shiga toxin, and a sequence encoding a verocytotoxin.

In some embodiments, the method comprises treating a disease or condition caused at least in part by the first microbial population. In some embodiments, the disease or condition is selected from the group consisting of inflammatory bowel disease, crohn's disease, ulcerative colitis, gastrointestinal infection, gastroenteritis, dysentery, kidney failure caused by hemolytic uremic syndrome, intra-abdominal infection, food sensitization, allergies, autism, and acne. In some embodiments, the disease or condition is a cancer. In some embodiments, the cancer is selected from the group consisting of melanoma, non-small cell lung cancer, renal cell carcinoma, urothelial carcinoma, colorectal cancer, hepatocellular cancer, stomach cancer, large B cell lymphoma, cervical cancer, small cell lung cancer, esophageal cancer, endometrial carcinoma, cutaneous squamous cell carcinoma, breast cancer, Hodgkin lymphoma, non-Hodgkin lymphoma, head & neck cancer, Merkel cell carcinoma, microsatellite instability (MSI)-high/deficient mismatch repair (dMMR) tumors, tumor mutational burden (TMB)-high tumors, and any other tumor types approved to be treated with immunotherapies (e.g., immune checkpoint inhibitors). In some embodiments, the method further comprises contacting the population with an additional therapeutic. In some embodiments, the additional therapeutic comprises an antibiotic peptide or a biologically active fragment thereof. In some embodiments, the additional therapeutic is selected from the group consisting of oral corticosteroids, oral aminosalicylates, TNF inhibitors, immunosuppressants, and integrin/integrin receptor antagonists. In some embodiments, the additional therapeutic is selected from the group consisting of 5-Fluorouracil, leucovorin, oxaliplatin, bevacizumab, panitumumab, cetuximab, capecitabine, irinotecan, ziv-aflibercept, ramucirumab, regorafenib, trifluridine, tipiracil, pembrolizumab, nivolumab, ipilimumab, atezolizumab, avelumab, durvalumab, cemiplimab-rwlc, trastuzumab, pertuzumab, lapatinib, encorafenib, Larotrectinib, entrectinib, and a biologically active fragment thereof. In some embodiments, the additional therapeutic comprises carbidopa-levodopa. In some embodiments, the additional therapeutic comprises an antidepressant. In some embodiments, the additional therapeutic is selected from the group consisting of salicylic acid, azelaic acid, dapsone (Aczone) gel, retinoids, isotretinoin anti-androgen agents, laser therapy, chemical peels, acne extraction techniques, and steroids. In some embodiments, the recombinant bacteriophage is lytic; wherein (i) the recombinant lytic bacteriophage replicates within the target bacterium prior to a microbial species dying, producing a replicated recombinant lytic bacteriophage; (ii) the replicated recombinant lytic bacteriophage is released from the microbial species following death of the microbial species; and (iii) the replicated recombinant lytic bacteriophage infects a microbial species that is not infected by the recombinant lytic bacteriophage. In some embodiments, the bacteriophage is administered intramuscularly, intravenously, subcutaneously, orally, transmucosally, buccally, sublingually, rectally, intranasally, or topically. In some embodiments, the first microbial population and the second microbial population are located in a region of a body selected from the group consisting of the gastrointestinal tract, skin, intraperitoneal cavity, colon, pancreas, bile duct, liver, and gall bladder.

In some embodiments, described herein is a pharmaceutical composition comprising the recombinant bacteriophage described herein, and a pharmaceutically acceptable excipient.

### BRIEF DESCRIPTION OF DRAWINGS

FIG. 1 shows data demonstrating specific killing of bacterial species Pseudomonas aeruginosa with the engineered bacteriophage. Panel 1 on the left (P1A and P1B) shows wild type bacteria. Panel 2 (middle, P2A, P2B) represent bacteria expressing mCherry and Panel 3 (right, P3A, P3B) represent a population of bacteria of the two types mixed 1:1. P1A, P2A and P3A represent normal bacterial growth in light contrast images without treatment with phage, whereas P1B, P2B and P3B are fluorescent images for detecting mCherry in the presence of indicated phages. Top rows of each, bacteria treated with wild-type bacteriophage (p1772). Middle rows, bacteria treated with phage containing a Cas system targeting the bacterial genome (p1772e005). Bottom row, bacteria treated with phage containing a Cas system targeting mCherry (p1772e081). Solid white arrowheads indicate presence of mCherry positive bacterial cells by observation and color image view. Hollow arrowheads represent reduced mCherry positive bacterial cells by observation and color image view.

FIG. 2 shows data demonstrating specific killing of bacterial species Pseudomonas aeruginosa with the engineered bacteriophage. Panel 1 on the left (P1A and P1B) shows wild type bacteria. Panel 2 (middle, P2A, P2B) represent bacteria expressing mCherry and a carbenicillin resistant gene and Panel 3 (right, P3A, P3B) represent a population of bacteria of the two types mixed 1:1. The bacteria and phage were mixed at an approximate MOI of 1 and plated immediately on either LB or carbenicillin containing plates. P1A, P2A and P3A represent normal bacterial growth in light contrast images without treatment with phage, whereas P1B, P2B and P3B are fluorescent images for detecting mCherry in the presence of indicated phages. Top rows of each, bacteria treated with wild-type bacteriophage (p1772). Middle rows, bacteria treated with phage containing a Cas system targeting the bacterial genome (p1772e005). Bottom row, bacteria treated with phage containing a Cas system targeting mCherry (p1772e081). Solid white arrowheads indicate presence of mCherry positive bacterial cells by observation and color image view. Hollow arrowheads represent reduced mCherry positive bacterial cells by observation and color image view.

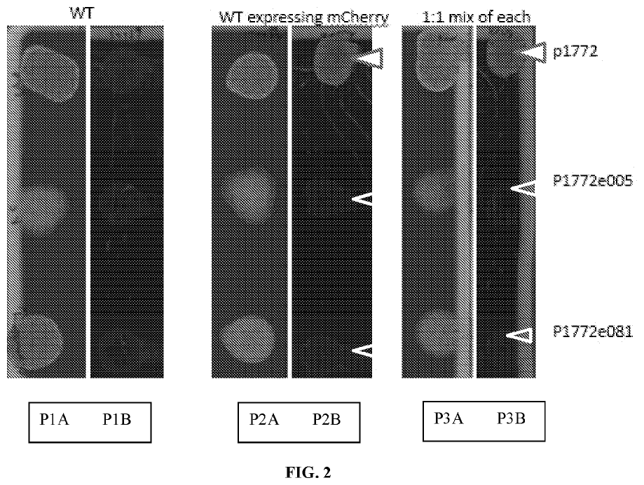

### DETAILED DESCRIPTION

### Certain Terminology

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

Unless the context indicates otherwise, it is specifically intended that the various features of the disclosure described herein are able of being used in any combination. Moreover, the present disclosure also contemplates that in some embodiments, any feature or combination of features set forth herein are excluded or omitted. To illustrate, if the specification states that a composition comprises components A, B and C, it is specifically intended that any of A, B or C, or a combination thereof, are omitted and disclaimed singularly or in any combination.

One of skill in the art will understand the interchangeability of terms designating the various CRISPR-Cas systems and their components due to a lack of consistency in the literature and an ongoing effort in the art to unify such terminology.

As used in the description and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about” as used herein when referring to a measurable value such as a dosage or time period and the like refers to variations of ±20%, ±10%, ±5%, ±1%, ±0.5%, or even ±0.1% of the specified amount. As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise”, “comprises”, and “comprising”, “includes”, “including”, “have” and “having”, as used herein, specify the presence of the stated features, steps, operations, elements, and/or components, but do not preclude the presence or addition of one or more other features, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

The term “consists of” and “consisting of”, as used herein, excludes any features, steps, operations, elements, and/or components not otherwise directly stated. The use of “consisting of” limits only the features, steps, operations, elements, and/or components set forth in that clause and does exclude other features, steps, operations, elements, and/or components from the claim as a whole.

The terms “complementary” or “complementarity”, as used herein, refer to the natural binding of polynucleotides under permissive salt and temperature conditions by base-pairing. For example, the sequence “A-G-T” binds to the complementary sequence “T-C-A.” Complementarity between two single-stranded molecules is “partial,” in which only some of the nucleotides bind, or it is complete when total complementarity exists between the single stranded molecules. The degree of complementarity between nucleic acid strands has significant effects on the efficiency and strength of hybridization between nucleic acid strands.

“Complement” as used herein means 100% complementarity or identity with the comparator nucleotide sequence or it means less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity). Complement or complementable may also be used in terms of a “complement” to or “complementing” a mutation.

As used herein, the phrase “substantially identical,” or “substantial identity” in the context of two nucleic acid molecules, nucleotide sequences or protein sequences, ref ers to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refers to two or more sequences or subsequences that have at least about 70%, at least about 75%, at least about 80%, at least about 85%, at least about 90%, at least about 95, 96, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some instances, “Percent identity” is determined using BLASTX version 2.0 for translated nucleotide sequences and BLASTN version 2.0 for polynucleotide sequences.

As used herein, the term “CRISPR phage”, “CRISPR enhanced phage”, and “crPhage” refers to a bacteriophage particle comprising bacteriophage DNA comprising at least one heterologous polynucleotide that encodes at least one component of a CRISPR-Cas system (e.g., CRISPR array, crRNA; e.g., P1 bacteriophage comprising an insertion of a targeting crRNA). In some embodiments, the polynucleotide encodes at least one transcriptional activator of a CRISPR-Cas system. In some embodiments, the polynucleotide encodes at least one component of an anti-CRISPR polypeptide of a CRISPR-Cas system.

As used herein, a “target nucleotide sequence” refers to the portion of a target gene (i.e., target region in the genome or the “protospacer sequence,” which is adjacent to a protospacer adjacent motif (PAM) sequence) that is fully complementary or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a CRISPR array.

As used herein, the term “protospacer adjacent motif” or “PAM” refers to a DNA sequence present on the target DNA molecule adjacent to the nucleotide sequence matching the spacer sequence. This motif is found in the target gene next to the region to which a spacer sequence binds as a result of being complementary to that region and identifies the point at which base pairing with the spacer nucleotide sequence begins. The exact PAM sequence that is required varies between each different CRISPR-Cas system and is identified through established bioinformatics and experimental procedures. Non-limiting examples of PAMs include CCA, CCT, CCG, CCT, CCA, TTC, AAG, AGG, ATG, GAG, and/or CC. In some instances, in Type I systems, the PAM is located immediately 5′ to the sequence that matches the spacer, and thus is 3′ to the sequence that base pairs with the spacer nucleotide sequence, and is directly recognized by Cascade. In some instances, for B. halodurans Type I-C systems, the PAM is YYC, where Y can be either T or C. In some instances, for the P. aeruginosa Type I-C system, the PAM is TTC.

Once a cognate protospacer and PAM are recognized, Cascade generally recruits the endonuclease Cas3, which then cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a—protospacer adjacent motif recognition domain at the C-terminus of Cas9).

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes include both coding and non-coding regions (e.g., introns, regulatory elements, promoters, enhancers, termination sequences and/or 5′ and 3′ untranslated regions). A gene is “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, the term “CRISPR phage”, “CRISPR enhanced phage”, and “crPhage” refers to a bacteriophage particle comprising bacteriophage DNA comprising at least one heterologous polynucleotide. In some embodiments, the polynucleotide encodes at least one component of a CRISPR-Cas system (e.g., CRISPR array, crRNA, or Cascade; e.g., bacteriophage comprising an insertion of crRNA targeting).

By the terms “treat,” “treating,” or “treatment,” it is intended that the severity of the subject's condition is reduced or at least partially improved or modified and that some alleviation, mitigation or decrease in at least one clinical symptom is achieved, and/or there is a delay in the progression of the disease or condition, and/or delay of the onset of a disease or illness. With respect to an infection, a disease or a condition, the term refers to a decrease in the symptoms or other manifestations of the infection (including bacterial burden in the subject's tissues), disease or condition. In some embodiments, treatment provides a reduction in symptoms or other manifestations of the infection, disease or condition by at least about 5%, e.g., about 10%, 15%, 20%, 25%, 30%, 35%, 40%, 45%, 50% or more.

The terms “prevent,” “preventing,” and “prevention” (and grammatical variations thereof) refer to prevention and/or delay of the onset of an infection, disease, condition and/or a clinical symptom(s) in a subject and/or a reduction in the severity of the onset of the infection, disease, condition and/or clinical symptom(s) relative to what would occur in the absence of carrying out the methods disclosed herein prior to the onset of the disease, disorder and/or clinical symptom(s). Thus, in some embodiments, to prevent infection, food, surfaces, medical tools and devices are treated with compositions and by methods disclosed herein.

In some instances, in Type I systems, the PAM is located immediately 5′ to the sequence that matches the spacer, and thus is 3′ to the sequence that base pairs with the spacer nucleotide sequence, and is directly recognized by Cascade. In some instances, for B. halodurans Type I-C systems, the PAM is YYC, where Y can be either T or C. In some instances, for the P. aeruginosa Type I-C system, the PAM is TTC. Once a cognate protospacer and PAM are recognized, Cascade generally recruits the endonuclease Cas3 is recruited, which then cleaves and degrades the target DNA. For Type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a—protospacer adjacent motif recognition domain at the C-terminus of Cas9).

The terms “individual”, or “subject” as used herein includes any animal that has or is susceptible to an infection, disease or condition involving bacteria. Thus, in some embodiments, subjects are mammals, avians, reptiles, amphibians, fish, crustaceans, or mollusks. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets (e.g., parakeets, parrots, macaws, cockatoos, canaries, and the like). Fish subjects include but are not limited to species used in aquaculture (e.g., tuna, salmon, tilapia, catfish, carp, trout, cod, bass, perch, snapper, and the like). Crustacean subjects include but are not limited to species used in aquaculture (e.g., shrimp, prawn, lobster, crayfish, crab and the like). Mollusk subjects include but are not limited to species used in aquaculture (e.g., abalone, mussel, oyster, clams, scallop and the like). In some embodiments, suitable subjects include both males and females and subjects of any age, including embryonic (e.g., in-utero orin-ovo), infant, juvenile, adolescent, adult and geriatric subjects. In some embodiments, a subject is a human.

As used here the term “isolated” in context of a nucleic acid sequence is a nucleic acid sequence that exists apart from its native environment.

As used herein, “expression cassette” means a recombinant nucleic acid molecule comprising a nucleotide sequence of interest (e.g., the recombinant nucleic acid molecules and CRISPR arrays disclosed herein), wherein the nucleotide sequence is operably associated with at least a control sequence (e.g., a promoter).

As used herein, “chimeric” refers to a nucleic acid molecule or a polypeptide in which at least two components are derived from different sources (e.g., different organisms, different coding regions).

As used herein, “selectable marker” means a nucleotide sequence that when expressed imparts a distinct phenotype to the host cell expressing the marker and thus allows such transformed cells to be distinguished from those that do not have the marker.

As used herein, “vector” refers to a composition for transferring, delivering or introducing a nucleic acid (or nucleic acids) into a cell.

As used herein, “pharmaceutically acceptable” means a material that is not biologically or otherwise undesirable, i.e., the material is administered to a subject without causing any undesirable biological effects such as toxicity.

As used herein the term “biofilm” means an accumulation of microorganisms embedded in a matrix of polysaccharide. Biofilms form on solid biological or non-biological surfaces and are medically important, accounting for over 80 percent of microbial infections in the body.

As used herein, the term “commensal” can mean a microbial species or strain that does not cause an undesirable effect.

As used herein, the term “pathogenic” can mean a microbial species or strain that can cause an undesirable biological effect.

### Target Bacterium

In some embodiments, disclosed herein is a recombinant bacteriophage comprising an exogenous antimicrobial agent. In some embodiments, disclosed herein is a recombinant bacteriophage comprising a first nucleic acid sequence encoding an exogenous antimicrobial agent. In certain aspects, disclosed herein is a recombinant bacteriophage comprising a first nucleic acid sequence encoding an exogenous antimicrobial agent that targets a first microbial population to allow a second microbial population to thrive. In certain aspects disclosed herein is a recombinant bacteriophage encoding an exogenous antimicrobial agent, wherein the exogenous antimicrobial agent targets a first microbial population and not a second microbial population; wherein the first microbial population comprises a genomic sequence associated with a pathogenic response and the second microbial population does not comprise the genomic sequence. In some embodiments, the second microbial population is associated with improved health outcomes.

In some embodiments, a “first microbial population” is a “first microbe”, and vice versa. In some embodiments, a “second microbial population” is a “second microbe”, and vice versa.

In some embodiments, a “population” comprises a number of bacteria of the same group and/or species that reside in a particular niche of the microbiome. In some embodiments, a population of bacterial may not be genetically identical. In some embodiments, a population of bacteria may be at least 80% identical, 85% identical, 90% identical, 95% identical, 97.5% identical, or 99% identical. In some embodiments, a population comprises multiple strains of a microbial species. In some embodiments, a population comprises multiple sub-species, or strains of a microbial species.

In some embodiments, the exogenous antimicrobial agent targets a first microbial population to allow a second microbial population to thrive. In some embodiments, allowing the second microbial population to thrive comprises the second microbial population replicating at an increased rate compared to the first microbial population. In some embodiments, the proportion of the microbiome that is the second microbial population increases following introduction of the antimicrobial agent. In some embodiments, the proportion of the microbiome that is the second microbial population relative to the other microbial population in the microbiome increases by at least about 10%, 20%, 30% 40%, 50%, 60%, 70%, 80%, 90%, 100%, 120%, 140%, 160%, 180%, 200%, or more than 200%.

In certain embodiments, disclosed herein is a recombinant bacteriophage comprising a first nucleic acid sequence encoding an exogenous antimicrobial agent that targets a first microbial population, wherein targeting the first microbial population alters the balance between the first microbial population and the second microbial population. In some embodiments, the population balance is altered by inhibiting the growth of a first microbial population and a second microbial population, wherein the first microbial population is inhibited at a higher rate than the second microbial population. In some embodiments, the population balance is altered by killing the bacteria comprising the first microbial population and the second microbial population, wherein the bacteria comprising first microbial population is killed at a higher rate than the bacteria comprising the second microbial population. In some embodiments, the population balance is altered by completely killing the first microbial population.

In some embodiments, the population balance is altered by preferentially reducing the population of a first microbial population. In some embodiments, preferentially reducing the population of a first microbial population confers a growth advantage on the second microbial population. In some embodiments, preferentially reducing the population of a first microbial population results in competitive exclusion of the first microbial population by the second microbial population. In some embodiments, preferentially reducing the population of a first microbial population results in competitive exclusion of the first microbial population by a third microbial population. In some embodiments, preferentially reducing the population of a first microbial population results in the second microbial population suppressing the first microbial population. In some embodiments, preferentially reducing the population of a first microbial population results in a third microbial population suppressing the first microbial population.

In some embodiments, the population balance is altered by causing the first microbial population to become growth constrained. In some embodiments, this confers a growth advantage to the second microbial population. In some embodiments, this allows the second microbial population to outcompete the first microbial population. In some embodiments, this allows the second microbial population to grow into the first microbial population's niche. In some embodiments, this allows a third microbial population to grow into the first microbial population's niche.

In some embodiments, the rate of growth of the second microbial population increased following introduction of the antimicrobial agent when compared to the rate of growth of the first microbial population. In some embodiments, the rate of growth increases by at least about 10%, 20%, 30% 40, %, 50%, 60%, 70%, 80%, 90%, 100%, 120%, 140%, 160%, 180%, 200%, or more than 200%. In some embodiments, the rate at which the first microbial population is killed by the exogenous antimicrobial agent is greater than the rate at which the second microbial population is killed by the exogenous antimicrobial agent. In some embodiments, the rate at which the first microbial population is killed by the exogenous antimicrobial agent is greater than the rate at which the second microbial population is killed by the exogenous antimicrobial agent is greater by at least about 10%, 20%, 30%40, %, 50%, 60%, 70%, 80%, 90%, 100%, 120%, 140%, 160%, 180%, 200%, or more than 200%.

In some embodiments, the ratio at which the antimicrobial agent kills the first microbial population with respect to the second microbial population is at least about 2:1, 3:1, 4:1, 5:1, 6:1, 7:1, 8:1, 10:1 15:1, 20:1, 30:1, 40:1, 80:1, 100:1, 1000:1, 10,000:1, or more than 100,000:1. In some embodiments, the ratio at which the antimicrobial agent kills the first microbial population with respect to the second microbial population is at most about 2:1, 3:1, 4:1, 5:1, 6:1, 7:1, 8:1, 10:1 15:1, 20:1, 30:1, 40:1, 80:1, 100:1, 1000:1, 10,000:1, or 100,000:1.

In some embodiments, the first microbial population and the second microbial population are the same genus of bacteria. In some embodiments, the first microbial population and the second microbial population are the same species of bacteria. In some embodiments, the first microbial population and the second microbial population are the same strain of bacteria. In some embodiments, the first microbial population and the second microbial population are different sub-strains of bacteria. In some embodiments, the first microbial population and the second microbial population are different strains of bacteria. In some embodiments, the first microbial population and the second microbial population are different. In some embodiments, the first microbial population and the second microbial population are different genus of bacteria.

In some embodiments, the genome of the first microbial population and the second microbial population are at least about 80% identical, 85% identical, 90% identical, 95% identical, 97.5% identical, or 99% identical.

In some embodiments, the first microbial population (or first microbe) comprises a species selected from the group consisting of Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Ehrlichia chaffeensis, Ehrlichia ewingii, Eikenella corrodens, Enterobacter cloacae, Enterococcus avium, Enterococcus durans, Enterococcus faecalis, Enterococcus faecium, Enterococcus gallinarum, Enterococcus maloratus, Escherichia coli, Fusobacterium necrophorum, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Leishmania donovani, Leptospira interrogans, Leptospira noguchii, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Pasteurella multocida, Pasteurella tularensis, Peptostreptococcus, Porphyromonas gingivalis, Prevotella melaninogenica, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Ureaplasma urealyticum, Vibrio cholerae, Vibrio comma, Vibrio parahaemolyticus, Vibrio vulnificus, Yersinia enterocolitica, Yersinia pestis, and Yersinia pseudotuberculosis. In some embodiments, the first microbial population comprises E. coli. In some embodiments, the E. coli comprises an adherent-invasive E. coli (AIEC) strain. In some embodiments, the E. coli comprises apks+genomic sequence. In some embodiments, the E. coli comprises a shiga toxin-producing E. coli (STEC) (e.g., E. coli 0157), verocytotoxin-producing E. coli (VTEC), or enterohemorrhagic E. coli (EHEC) strains. In some embodiments, the E. coli is a multidrug-resistant (MDR) strain. In some embodiments, the E. coli is an extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the first microbial population comprises Bacteroides. In some, embodiments, the Bacteroides species comprises B. fragilis, or B. thetaiotaomicron. In some embodiments, the B. fragilis comprises an enterotoxigenic B. fragilis. In some embodiments, the first microbial population comprises Enterococcus. In some embodiments, the Enterococcus species comprises E. faecalis, or E. faecium. In some embodiments, the Enterococcus species comprises vancomycin-resistant E. faecalis, or vancomycin-resistant E. faecium. In some embodiments, the first microbial population comprises Shigella. In some embodiments, the first microbial population comprises Hafnia alvei. In some embodiments, the first microbial population comprises Pseduomonas aeruginosa. In some embodiments, the first microbial population comprises Mongsnella morganii. In some embodiments, the first microbial population comprises Pseudomonas putida. In some embodiments, the first microbial population comprises Citrobacter koseri. In some embodiments, the first microbial population comprises Klebsiella pneumonia. In some embodiments, the first microbial population comprises Campylobacter jejuni. In some embodiments, the first microbial population comprises Mycobacterium avium subspecies paratuberculosis.

In some embodiments, the second microbial population (or second microbe) comprises a species selected from the group consisting of Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetii, Ehrlichia chaffeensis, Ehrlichia ewingii, Eikenella corrodens, Enterobacter cloacae, Enterococcus avium, Enterococcus durans, Enterococcus faecalis, Enterococcus faecium, Enterococcus gallinarum, Enterococcus maloratus, Escherichia coli, Fusobacterium necrophorum, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Leishmania donovani, Leptospira interrogans, Leptospira noguchii, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Pasteurella multocida, Pasteurella tularensis, Peptostreptococcus, Porphyromonas gingivalis, Prevotella melaninogenica, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Ureaplasma urealyticum, Vibrio cholerae, Vibrio comma, Vibrio parahaemolyticus, Vibrio vulnificus, Yersinia enterocolitica, Yersinia pestis, and Yersinia pseudotuberculosis. In some embodiments, the second microbial population comprises E. coli. In some embodiments, the E. coli is a commensal E. coli. In some embodiments, the second microbial population comprises Bacteroides. In some embodiments, the B. fragilis comprises a commensal B. fragilis. In some embodiments, the second microbial population comprises Enterococcus. In some embodiments, the Enterococcus species is commensal. In some embodiments, the commensal Enterococcus species is sensitive to vancomycin. In some embodiments, the second microbial population compresses Shigella. In some embodiments, the second microbial population comprises Hafnia alvei. In some embodiments, the second microbial population comprises Pseduomonas aeruginosa. In some embodiments, the second microbial population comprises Mongsnella morganii. In some embodiments, the second microbial population comprises Pseudomonas putida. In some embodiments, the second microbial population comprises Citrobacter koseri. In some embodiments, the second microbial population comprises Klebsiella pneumonia. In some embodiments, the second microbial population comprises Campylobacter jejuni. In some embodiments, the second microbial population comprises Mycobacterium avium subspecies paratuberculosis.

In some embodiments, the first microbial population comprises an adherent-invasive E. coli strain and the second microbial population comprises a commensal E. coli strain. In some embodiments, the first microbial population comprises an E. coli strain comprising a nucleic acid comprising pks+sequences, and the second microbial population comprises a commensal E. coli strain. In some embodiments, the first microbial population comprises an enterotoxigenic B. fragilis strain, and the second microbial population comprises a commensal B. fragilis strain. In some embodiments, the first microbial population is selected from the group consisting of a shiga toxin-producing E. coli (STEC) (e.g., E. coli 0157), verocytotoxin-producing E. coli (VTEC), or enterohemorrhagic E. coli (EHEC) strains, and the second microbial population comprises a commensal E. coli strain. In some embodiments, the first microbial population comprises a Shigella spp., and the second microbial population comprises a commensal E. coli strain. In some embodiments, the first microbial population comprises Enterococcus spp., and the second microbial population comprises a different Enterococcus spp. In some embodiments, the first Enterococcus spp. is E. faecalis or E. faecium. In some embodiments, the first Enterococcus spp. is vancomycin-resistant E. faecalis or vancomycin-resistant E. faecium. In some embodiments, the first microbial population comprises a enteropathogenic E. coli (EPEC) strain, and the second microbial population comprises a commensal E. coli strain. In some embodiments, the first microbial population comprises a first Bacteroides spp., and the second microbial population comprises a second Bacteroides spp. In some embodiments, the first Bacteroides spp. is selected from the group consisting of B. fragilis, and B. thetaiotomicron. In some embodiments, the first microbial population comprises a species of Cutibacterium acnes that leads to acne and skin inflammation, and the second microbial population comprises a species of C. acnes that promotes healthy skin. In some embodiments, the first microbial population comprises a microbial species found in a gut microbiome of a formula-fed infant, and the second microbial population comprises a microbial species found in a gut microbiome of a breast-fed infant. In some embodiments, the first microbial population comprises a first species of Campyobacter jejuni, and the second microbial population comprises a second species of Campyobacter jejuni, wherein the first species is associated with anxiety. In some embodiments, the first microbial population is associated with elevated levels of IgM and IgA in a subject, relative to the second microbial population. In some embodiments, the first microbial population causes elevated levels of IgM and IgA in a subject, relative to the second microbial population. The following Table (TABLE 1) summarizes example microbial populations that are selectively targeted using compositions and methods disclosed herein.

TABLE 1
Exemplary enteric bacterial populations

| | Functional | Second Microbial | Functional |
|---|---|---|---|
| First Microbial Population | characteristic | Population | characteristic |
| Adherent-invasive E. coli | Adheres and | Commensal E. coli strain | Does not |
| strain LF82 | invades intestinal | ATCC 10798 | |
| | epithelial cells | | |
| E. coli pks⁺ strain ATCC | Produces | Commensal E. coli strain | Does not |
| 25922 | colibactin | ATCC 10798 | |
| E. coli (STEC) (e.g., E. coli | Produces Shiga | Commensal E. coli strain | Does not |
| O157:H7) ATCC 35150 | toxin | ATCC 10798 | |
| E. coli (VTEC) | Producing | Commensal E. coli strain | Does not |
| | Verocytotoxin | ATCC 10798 | |
| E. coli (EHEC) strain BAA- | Enterohemorrhagic | Commensal E. coli strain | Does not |
| 2326 | | ATCC 10798 | |
| Shigella dysenteriae ATCC | Produces Shiga | Commensal E. coli strain | Does not |
| 13313 | toxin | ATCC 10798 | |
| Enterococcus faecalis | Vancomycin | A probiotic E. faecalis | Does not |
| OG1RF | resistant | strain Symbioflor | |
| Enterococcus faecium C68 | Vancomycin | A commensal E. faecium | Does not |
| | resistant | strain Com12 | |
| Bacteriodes fragilis ATCC | Releases toxin, | Commensal B. fragilis | Does not |
| 43858 | e.g., fragilysin | ATCC 25285 | |
| Cutibacterium acnes strain | Causes acne and | Cutibacterium acnes strain | Does not |
| ATCC 6919 | skin inflammation | ATCC 11828 | cause acne |
| | | | and skin |
| | | | inflammation |

Adherent-invasive E. coli (AIEC) strains are a strain of E. coli that strongly adheres to and invades intestinal epithelial cells. In some embodiments, the first microbial population comprises an AIEC. In some embodiments, the AIEC may be located in the gastrointestinal tract of a subject. In some embodiments, the AIEC may be located in the small or large intestine of a subject. In some embodiments, the AIEC may be located in the ilium of a subject. In some embodiments, the subject has Inflammatory Bowel Disease. In some embodiments, the subject has Crohn's Disease, or ulcerative colitis. In some embodiments, the genomic sequence associated with the pathogenic response is present in an AIEC strain of E. coli and not in a second microbial population. In some embodiments, the second microbial population is a commensal strain of E. coli. In some embodiments, a bacteria comprises a genomic sequence that comprises a protein coding sequence, or a regulatory sequence. In some embodiments, a bacteria comprises a genomic sequence that comprises a gene derived from Salmonella or Yersinia. In some embodiments, a bacteria comprises a genomic sequence that comprises a sequence associated with adherence or invasion. In some embodiments, a bacteria comprises a genomic sequence that comprises long polar fimbriae (LPF).

In some embodiments, the first microbial population comprises apks+strain of E. coli. In some embodiments, the pks+strain of E. coli may be located in the gastrointestinal tract of a subject. In some embodiments, the pks+strain of E. coli may be located in the large or small intestine of a subject. In some embodiments, the subject may have colorectal cancer. In some embodiments, the genomic sequence associated with the pathogenic response is present in a pks+strain of E. coli, and not in a second microbial population. In some embodiments, the second microbial population is a commensal strain of E. coli. In some embodiments, a bacteria comprises a genomic sequence that comprises a protein coding sequence, or a regulatory sequence. In some embodiments, a bacteria comprise a genomic sequence that is apks genomic island, or a gene encoding an enzyme that is involved in the synthesis of colibactin.

In some embodiments, enterotoxigenic Bacteroides fragilis secretes enterotoxins including B. fragilis toxin (BFT) or fragilysin, and is associated with acute inflammatory responses in intestinal mucosa. In some embodiments, the first microbial population comprises an enterotoxigenic B. fragilis. In some embodiments, the enterotoxigenic B. fragilis may be located in the gastrointestinal tract of a subject. In some embodiments, the enterotoxigenic B. fragilis may be located in the large or small intestine of a subject. In some embodiments, the subject has colorectal cancer. In some embodiments, the genomic sequence associated with the pathogenic response is present in an enterotoxigenic B. fragilis, and not in a second microbial population. In some embodiments, the second microbial population comprises a commensal strain of B. fragilis. In some embodiments, a bacteria comprises a genomic sequence that comprises a protein coding sequence, or a regulatory sequence. In some embodiments, a bacteria comprises a genomic sequence that is a sequence encoding an enterotoxin. In some embodiments, a bacteria comprises a genomic sequence that is a sequence encoding BFT or fragilysin.

In some embodiments, the first microbial population comprises a pathogenic E. coli strain. In some embodiments, the pathogenic E. coli strain comprises a shiga toxin-producing E. coli (STEC) (e.g., E. coli 0157), verocytotoxin-producing E. coli (VTEC), or enterohemorrhagic E. coli (EHEC) strains. In some embodiments, the pathogenic strain of E. coli may be located in the gastrointestinal tract of a subject. In some embodiments, the pathogenic strain of E. coli may be located in the large or small intestine of a subject. In some embodiments, the subject may have a gastrointestinal infection, gastroenteritis, dysentery, or kidney failure caused by hemolytic uremic syndrome. In some embodiments, a bacteria comprises a genomic sequence associated with the pathogenic response and is present in a pathogenic strain of E. coli, and not in a second microbial population. In some embodiments, the second microbial population comprises a commensal strain of E. col. In some embodiments, a bacteria comprises a genomic sequence that comprises a protein coding sequence, or a regulatory sequence. In some embodiments, a bacteria comprises a genomic sequence that is a toxin sequence. In some embodiments, a toxin sequence is a shiga toxin. In some embodiments, a toxin sequence is a verocytotoxin sequence.

Shigella is a gram-negative genus of bacteria closely related to E. col. In some embodiments, the first microbial population comprises a Shigella spp. In some embodiments, the Shigella spp. may be located in the gastrointestinal tract of a subject. In some embodiments, the Shigella spp. may be located in the large or small intestine of a subject. In some embodiments, the subject may have gastrointestinal infection, gastroenteritis, dysentery, or kidney failure caused by hemolytic uremic syndrome. In some embodiments, a bacteria comprises a genomic sequence associated with a pathogenic response, and is present in a Shigella spp., and not in a second microbial population. In some embodiments, the second microbial population comprises a commensal strain of E. col. In some embodiments, a bacteria comprises a genomic sequence that comprises a protein coding sequence, or a regulatory sequence.

Enterococcus is a gram-positive genus of the phylum Firmicutes, including E. faecalis and E. faecium, associated with human infections. In some embodiments, the first microbial population comprises Enterococcus spp. In some embodiments, the Enterococcus spp. may be E. faecalis or E. faecium. In some embodiments, the E. faecalis or E. faecium may be resistant to vancomycin. In some embodiments, the Enterococcus spp. may be located in the intraperitoneal cavity, small intestine, colon, pancreas, bile duct, liver, gallbladder, or bloodstream of a subject. In some embodiments, the subject may have an intra-abdominal infection. In some embodiments, the intra-abdominal infection comprises peritonitis, diverticulitis, cholecystitis, cholangitis, pancreatitis, or any combination thereof. In some embodiments, the subject may have a bloodstream infection. In some embodiments, the bloodstream infection comprises bacteremia, sepsis, or endocarditis. In some embodiments, a bacteria comprises a genomic sequence associated with a pathogenic response, and is present in an Enterococcus spp., and not in a second microbial population. In some embodiments, a bacteria comprises a genomic sequence associated with a pathogenic response, and is present in E. faecalis or E. faecium, and not in a second microbial population. In some embodiments, a bacteria comprises a genomic sequence associated with a antibiotic response, and is present in E. faecalis or E. faecium, and not in a second microbial population. In some embodiments, the second microbial population comprises a commensal strain of Enterococcus spp. In some embodiments, a bacteria comprises a genomic sequence that comprises a protein coding sequence, or a regulatory sequence.

In some embodiments, the first microbial population comprises an enteropathogenic E. coli (EPEC) strain. In some embodiments, the EPEC may be located in the gastrointestinal tract of a subject. In some embodiments, the EPEC may be located in the large or small intestine of a subject. In some embodiments, the subject may have gastrointestinal infection, gastroenteritis, or Parkinson's disease. In some embodiments, the genomic sequence associated with the pathogenic response is present in an EPEC, and not in a second microbial population. In some embodiments, the second microbial population comprises a commensal strain of E. coli. In some embodiments, the genomic sequence comprises a protein coding sequence, or a regulatory sequence.

In some embodiments, the first microbial population comprises a Bacteroides spp. In some embodiments, the Bacteroides spp. comprises B. fragilis or B. thetaiotamicron. In some embodiments, the Bacteroides spp. may be located in the gastrointestinal tract of a subject. In some embodiments, the Bacteriodes spp. may be located in the large or small intestine of a subject. In some embodiments, the subject may have an intra-abdominal infection. In some embodiments, the subject is at risk for developing an intra-abdominal infection. In some embodiments, the intra-abdominal infection comprises peritonitis, diverticulitis, cholecystitis, cholangitis, pancreatitis, or any combination thereof. In some embodiments, the subject has a cancer or a tumor. In some embodiments, the subject is at risk for developing a cancer or a tumor. In some embodiments, the cancer or tumor comprises melanoma, non-small cell lung cancer, renal cell carcinoma, urothelial carcinoma, colorectal cancer, hepatocellular cancer, stomach cancer, large B cell lymphoma, cervical cancer, small cell lung cancer, esophageal cancer, endometrial carcinoma, cutaneous squamous cell carcinoma, breast cancer, Hodgkin lymphoma, non-Hodgkin lymphoma, head & neck cancer, Merkel cell carcinoma, microsatellite instability (MSI)-high/deficient mismatch repair (dMMR) tumors, tumor mutational burden (TMB)-high tumors, and any other tumor types approved to be treated with immunotherapies (e.g., immune checkpoint inhibitors). In some embodiments, the genomic sequence associated with the pathogenic response is present in a Bacteroides spp., and not in a second microbial population. In some embodiments, the genomic sequence associated with the pathogenic response is present in a B. fragilis or B. thetaiotamicron, and not in a second microbial population. In some embodiments, the second microbial population comprises a commensal strain of Bacteroides spp. In some embodiments, the genomic sequence comprises a protein coding sequence, or a regulatory sequence.

In some embodiments, the C. acnes may be located on the skin of a subject. In some embodiments, the subject may have acne vulgaris. In some embodiments, the subject may be at risk for developing acne vulgaris. In some embodiments, the first microbial population comprises Cutibacterium acnes. In some embodiments, the first microbial population comprises C. acnes that contributes to acne or skin inflammation in a subject. In some embodiments, the genomic sequence associated with the pathogenic response is present in the C. acnes that contributes to acne or skin inflammation in a subject, and not in a second microbial population. In some embodiments, the second microbial population comprises a commensal strain of C. acnes. In some embodiments, the second microbial population comprises a commensal species that is not C. acnes. In some embodiments, the genomic sequence comprises a protein coding sequence, or a regulatory sequence.

The development of the gut microbiome early in life has been found to impact many health conditions, including allergies and autism. Breast-fed infants and formula-fed infants have different compositions of their microbiome. For example, breast-fed infants were found to have lower levels of gut microbe diversity, and a higher representation of infant-type Bipdobacteria compared to formula-fed infants prior to 6 months of age. Bifdobacteria have been associated with a diminished risk of allergic diseases, excessive weight gain, lactose intolerance, and rotavirus infections, while also being correlated with improved immune response to vaccines. Breast-fed infants also were found to have lower levels of Veillonellaceae, Enterococcaceae, Streptococcaceae, and Lachnospiraceae bacterial species relative to formula-fed infants. A higher level of Streptococcus sp. has been found in patients suffering from type 1 diabetes. In some embodiments, the first microbial population comprises a microbial species found in the microbiome of a formula-fed infant. In some embodiments, the first microbial population may be located in the gastrointestinal tract of the subject. In some embodiments, the microbial species found in the microbiome of a formula-fed infant may be located in the large or small intestine of a subject. In some embodiments, the subject has or may develop food allergies or autism. In some embodiments, the genomic sequence associated with the microbial species found in the microbiome of a formula-fed infant is present in a first microbial population, and not in a second microbial population. In some embodiments, the second microbial population comprises a microbial species found in the gut microbiome of a breast-fed infant. In some embodiments, the genomic sequence comprises a protein coding sequence, or a regulatory sequence.

Certain strains of Campylobacter jejuni are associated with anxiety. In some embodiments, the first microbial population comprises a Campylobacter jejuni strain associated with anxiety. In some embodiments, the Campylobacter jejuni strain associated with anxiety may be located in the gastrointestinal tract of a subject. In some embodiments, the Campylobacter jejuni strain associated with anxiety may be located in the large or small intestine of a subject. In some embodiments, the subject may have anxiety, depression, or cognitive dysfunction. In some embodiments, the genomic sequence associated with the pathogenic response is present in a Campylobacter jejuni strain associated with anxiety, and not in a second microbial population. In some embodiments, the second microbial population comprises a commensal strain of Campylobacter jejuni. In some embodiments, the second microbial population comprises a commensal species that is not Campylobacter jejuni. In some embodiments, the genomic sequence comprises a protein coding sequence, or a regulatory sequence.

In some embodiments, patients with depression show increased IgM and IgA response in the presence of gram-negative commensals. In some embodiments, the first microbial population comprises a microbial species associated with elevated levels of IgM or IgA in a subject. In some embodiments, the second microbial population comprises a microbial species that is not associated with elevated levels of IgM or IgA in a subject. In some embodiments, the second microbial population comprises a microbial species that is associated with decreased levels of IgM or IgA in a subject. In some embodiments, the microbial species associated with elevated levels of IgM and IgA in a subject may be located in the gastrointestinal tract of the subject. In some embodiments, the microbial species associated with elevated levels of IgM and IgA in a subject may be located in the large or small intestine of the subject. In some embodiments, the subject may have depression or cognitive dysfunction. In some embodiments, the genomic sequence associated with the pathogenic response is present in a microbial species associated with elevated levels of IgM and IgA in a subject, and not in a second microbial population. In some embodiments, the second microbial population comprises a commensal strain of the first microbial population. In some embodiments, the genomic sequence comprises a protein coding sequence, or a regulatory sequence.

In some embodiments, Mycobacterium avium subspecies paratuberculosis is associated with Crohn's disease in humans. In some embodiments, the first microbial population comprises a Mycobacterium avium subspecies paratuberculosis (MAP). In some embodiments, Mycobacterium avium subspecies paratuberculosis (MAP) may be located in the gastrointestinal tract of a subject. In some embodiments, the Mycobacterium avium subspecies paratuberculosis (MAP) may be located in the large or small intestine of a subject. In some embodiments, the subject may have Crohn's Disease. In some embodiments, the subject may have an elevated risk of developing Crohn's Disease. In some embodiments, a bacteria comprises a genomic sequence associated with a pathogenic response and is present in a Mycobacterium avium subspecies paratuberculosis (MAP), and not in a second microbial population. In some embodiments, the second microbial population comprises a commensal strain. In some embodiments, a bacteria comprises a genomic sequence that comprises a protein coding sequence, or a regulatory sequence.

Table 2 provides example microbial populations for use with the compositions and methods of the present application. For instance, the bacteriophage of the present application selectively target and kill the first microbe, while not killing the second microbe. In some cases, the first microbe comprises a target DNA sequence that is lacking in the second microbe. Non-limiting example target DNA are provided in Table 2 and Table 3.

TABLE 2

| | | | | Example | | |
|---|---|---|---|---|---|---|
| | | | | condition/ | | |
| | | | | disease | | |
| | First | | | associated | | Second |
| First | microbe | | | with the | Second | microbe |
| microbe | sample | Example | Target | first | microbe | sample |
| examples | strain(s) | target gene | DNA | microbe | examples | strains |
| Adherent- | Strain LF82 | E. coli | ipaA | Inflammatory | Commensal E. | e.g., K12 |
| invasive E. | | invasion genes | STL77042 | bowel disease | coli | (ATCC |
| coli | | | ipaB | (IBD) | | 10798); |
| | | | AAM89568 | | | MG1655(K12 |
| | | | ipaD | | | derived F + |
| | | | ANY58568 | | | cured ATCC |
| | | | | | | 700926). |
| pks + E. coli | ATCC 25922 | Colibactin | clbR | IBD, colon | Commensal E. | |
| | | | QLD56020 | cancer | coli | |
| Enterotoxigenic | ATCC 43858 | B. fragilis | bft1 | Colon cancer, | Commensal | |
| B. fragilis | | toxin genes | AB026625 | IBD | B. fragilis | |
| | | | bft2 | | | |
| | | | AB026625 | | | |
| | | | bft3 | | | |
| | | | AB026624 | | | |
| Enterococcus | BAA-2573 | Vanomycin | vanA | UTI, soft | Commensal | Symbioflor 1, |
| faecalis | | resistance gene | ADM24920 | tissue | Enterococcus | e.g. some |
| | | | | infection, | faecalis | probiotic |
| | | | | bacteremia, | | strains |
| | | | | endocarditis | | |
| Enterococcus | C68, and/or | Vancomycin | vanA | UTI, soft | Commensal | Cylactin, |
| faecium | V583 (PMID: | resistance gene | UBU71938 | tissue | Enterococcus | Fargo688, |
| | 24141120) | | | infection, | faecium | Commensals |
| | | | | bacteremia, | | Com 12, |
| | | | | endocarditis | | Com 15; e.g., |
| | | | | | | some probiotic |
| | | | | | | strains |
| Cutibacterium | ATCC 6919 | | | Acne vulgaris | Cutibacterium | ATCC 11828 |
| acnes (type Ia) | (PMID: | | | | acnes (type II | |
| | 31086023) | | | | strain) | |

### Antimicrobial Agents and Peptides

In some embodiments, a bacteriophage disclosed herein can be and/or is further genetically modified to express an antibacterial peptide, a functional fragment of an antibacterial peptide, or a lytic gene. In some embodiments, a bacteriophage disclosed herein expresses at least one antimicrobial agent or peptide disclosed herein. In some embodiments, a bacteriophage disclosed herein comprises a nucleic acid sequence that encodes an enzybiotic, where the protein product of the nucleic acid sequence targets phage resistant bacteria. In some embodiments, the bacteriophage comprises nucleic acids, which encode enzymes which assist in breaking down or degrading biofilm matrix. In some embodiments, a bacteriophage disclosed herein comprises nucleic acids encoding Dispersin D aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, xylanase, or lyase. In some embodiments, the enzyme is selected from the group consisting of cellulases, such as glycosyl hydroxylase family of cellulases, such as glycosyl hydroxylase 5 family of enzymes also called cellulase A; polyglucosamine (PGA) depolymerases; colanic acid depolymerases, such as 1,4-L-fucodise hydrolase characterization of a 1,4-beta-fucoside hydrolase degrading colanic acid; depolymerizing alginase; DNase I; a biologically active fragment of any one of the above, or combinations thereof. In some embodiments, a bacteriophage disclosed herein secretes an enzyme, or a biologically active fragment of an enzyme disclosed herein.

In some embodiments, a bacteriophage disclosed herein induces the target bacterium to express and/or secrete an antimicrobial agent or peptide. In some embodiments, a bacteriophage disclosed herein induces the target bacterium to secrete and/or expresses an antibiotic such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, pazufloxacin, or any antibiotic disclosed herein, a biologically active fragment of any of these, or a combination thereof. In some embodiments, a bacteriophage disclosed herein comprises a nucleic acid sequence encoding an antibacterial peptide, expresses an antibacterial peptide, or secretes a peptide that aids or enhances killing of a target bacterium. In some embodiments, a bacteriophage disclosed herein comprises a nucleic acid sequence encoding a peptide, a nucleic acid sequence encoding an antibacterial peptide, induces the target bacterium to express an antibacterial peptide, or secretes a peptide that aids or enhances the activity of a CRISPR-Cas system.

### CRISPR/CAS Systems

In some embodiments, the antimicrobial agent comprises one or more components of a CRISPR-Cas system. CRISPR-Cas systems are naturally adaptive immune systems found in bacteria and archaea. The CRISPR system is a nuclease system involved in defense against invading phages and plasmids that provides a form of acquired immunity. There is a diversity of CRISPR-Cas systems based on the set of cas genes, and their phylogenetic relationship. There are at least six different types (I through VI) where Type I represents over 50% of all identified systems in both bacteria and archaea. In some embodiments, a Type I, Type II, Type II, Type IV, Type V, or Type VI CRISPR-Cas system is used herein.

Type I systems are divided into seven subtypes including: Type I-A, Type I-B, Type I-C, Type I-D, Type I-E, Type I-F, and Type I-U. Type I CRISPR-Cas systems include a multi-subunit complex called Cascade (for complex associated with antiviral defense), Cas3 (a protein with nuclease, helicase, and exonuclease activity that is responsible for degradation of the target DNA), and CRISPR array encoding crRNA (stabilizes Cascade complex and directs Cascade and Cas3 to DNA target). Cascade forms a complex with the crRNA, and the protein-RNA pair recognizes its genomic target by complementary base pairing between the 5′ end of the crRNA sequence and a predefined protospacer. This complex is directed to homologous loci of pathogen DNA via regions encoded within the crRNA, and protospacer-adjacent motifs (PAMs) within the pathogen genome. Base pairing occurs between the crRNA and the target DNA sequence, leading to a conformational change. In the Type I-E system, the PAM is recognized by the CasA protein within Cascade, which then unwinds the flanking DNA to evaluate the extent of base pairing between the target and the spacer portion of the crRNA. Sufficient recognition leads Cascade to recruit and activate Cas3. Cas3 then nicks the non-target strand, and begins degrading the strand in a 3′-to-5′ direction.

In the Type I-C system, the proteins Cas5, Cas8c, and Cas7 form the Cascade effector complex. Cas5 processes the pre-crRNA (which can take the form of a multi-spacer array, or a single spacer between two repeats) to produce individual crRNA(s) made up of a hairpin structure formed from the remaining repeat sequence and a linear spacer. The effector complex then binds to the processed crRNA, and scans DNA to identify PAM sites. In the Type I-C system, the PAM is recognized by the Cas8c protein, which then acts to unwind the DNA duplex. If the sequence 3′ of the PAM matches the crRNA spacer that is bound to the effector complex, a conformational change in the complex occurs, and Cas3 is recruited to the site. Cas3 then nicks the non-target strand, and begins degrading the DNA.

In some embodiments, the CRISPR-Cas system is endogenous to the target bacterium. In some embodiments, the CRISPR-Cas system is exogenous to the target bacterium. In some embodiments, the CRISPR-Cas system is a Type I CRISPR-Cas system. In some embodiments, the CRISPR-Cas system is a Type I-A CRISPR-Cas system. In some embodiments, the CRISPR-Cas system is a Type I-B CRISPR-Cas system. In some embodiments, the CRISPR-Cas system is a Type I-C CRISPR-Cas system. In some embodiments, the CRISPR-Cas system is a Type I-C CRISPR-Cas system from Pseudomonas aeruginosa (PAIC). In some embodiments, the CRISPR-Cas system is a Type I-D CRISPR-Cas system. In some embodiments, the CRISPR-Cas system is a Type I-E CRISPR-Cas system. In some embodiments, the CRISPR-Cas system is a Type I-E CRISPR-CAS system from Escherichia coli (ECIE). In some embodiments, the CRISPR-Cas system is a Type I-F CRISPR-Cas system. In some embodiments, the CRISPR-Cas system is a Type I-E CRISPR-CAS system from Escherichia coli (ECIF). In some embodiments, the CRISPR-Cas system is a Type I-U CRISPR-Cas system. In some embodiments, the CRISPR-Cas system is a Type II CRISPR-Cas system. In some embodiments, the CRISPR-Cas system is a Type III CRISPR-Cas system.

In some embodiments, the bacteriophage comprises an additional CRISPR-Cas class defined by a signature effector, wherein the signature effector comprises Cas9, Cas10, Cas11, Cas12, Cas13, Cas14, Cas3, Cpf1, Cas9, CasΦ, Cas12b, or Cas12c. In some embodiments, the signature effector comprises as subtype of Cas9, Cas10, Cas11, Cas12, Cas13, Cas14, Cas3, Cpf1, Cas9, CasΦ, Cas12b, or Cas12c.

In some embodiments, processing of a CRISPR-array disclosed herein includes, but is not limited to, the following processes: 1) transcription of the nucleic acid encoding a pre-crRNA; 2) recognition of the pre-crRNA by Cascade and/or specific members of Cascade, such as Cas6; and (3) processing of the pre-crRNA by Cascade or members of Cascade, such as Cas6, into mature crRNAs. In some embodiments, the mode of action for a Type I CRISPR system includes, but is not limited to, the following processes: 4) mature crRNA complexation with Cascade; 5) target recognition by the complexed mature crRNA/Cascade complex; and 6) nuclease activity at the target leading to DNA degradation.

### CRISPR Phages

Disclosed herein, in certain embodiments, are bacteriophage compositions comprising CRISPR-Cas systems and methods of use thereof.

Bacteriophages or “phages” represent a group of bacterial viruses and are engineered or sourced from environmental sources. Individual bacteriophage host ranges are usually narrow, meaning, phages are highly specific to one strain or few strains of a bacterial species, and this specificity makes them unique in their antibacterial action. Bacteriophages are bacterial viruses that rely on the host's cellular machinery to replicate. Bacteriophages are generally classified as virulent or temperate phages, depending on their lifestyle. Virulent bacteriophages, also known as lytic bacteriophages, can only undergo lytic replication. Lytic bacteriophages infect a host cell, undergo numerous rounds of replication, and trigger cell lysis to release newly made bacteriophage particles. In some embodiments, the lytic bacteriophages disclosed herein retain their replicative ability. In some embodiments, the lytic bacteriophages disclosed herein retain their ability to trigger cell lysis. In some embodiments, the lytic bacteriophages disclosed herein retain both they replicative ability and the ability to trigger cell lysis. In some embodiments, the bacteriophages disclosed herein comprise a CRISPR array. In some embodiments, the CRISPR array does not affect the bacteriophages ability to replicate and/or trigger cell lysis. Temperate or lysogenic bacteriophages can undergo lysogeny in which the phage stops replicating and stably resides within the host cell, either integrating into the bacterial genome, or being maintained as an extrachromosomal plasmid. Temperate phages can also undergo lytic replication similar to their lytic bacteriophage counterparts. Whether a temperate phage replicates lytically or undergoes lysogeny upon infection depends on a variety of factors including growth conditions, and the physiological state of the cell. A bacterial cell that has a lysogenic phage integrated into its genome is referred to as a lysogenic bacterium, or lysogen. Exposure to adverse conditions may trigger reactivation of the lysogenic phage, termination of the lysogenic state, and resumption of lytic replication by the phage. This process is called induction. Adverse conditions which favor the termination of the lysogenic state include desiccation, exposure to UV or ionizing radiation, and exposure to mutagenic chemicals. This leads to the expression of the phage genes, reversal of the integration process, and lytic multiplication. In some embodiments, the temperate bacteriophages disclosed herein are rendered lytic. The term “lysogeny gene” refers to any gene whose gene product promotes lysogeny of a temperate phage. Lysogeny genes can directly promote lysogeny, as in the case of integrase proteins that facilitate integration of the bacteriophage into the host genome. Lysogeny genes can also indirectly promote lysogeny, as in the case of CI transcriptional regulators which prevent transcription of genes required for lytic replication, and thus favor maintenance of lysogeny.

Bacteriophages package and deliver synthetic DNA using three general approaches. Under the first approach, the synthetic DNA is recombined into the bacteriophage genome in a targeted manner, which usually involves a selectable marker. Under the second approach, restriction sites within the phage are used to introduce synthetic DNA in-vitro. Under the third approach, a plasmid generally encoding the phage packaging sites and lytic origin of replication is packaged as part of the assembly of the bacteriophage particle. The resulting plasmids have been coined “phagemids.”

Phages are limited to a given bacterial strain for evolutionary reasons. In some cases, injecting their genetic material into an incompatible strain is counterproductive. Phages have therefore evolved to specifically infect a limited cross-section of bacterial strains. However, some phages have been discovered that inject their genetic material into a wide range of bacteria. The classic example is the P1 phage, which has been shown to inject DNA in a range of gram-negative bacteria.

Disclosed herein, in some embodiments, are bacteriophages comprising a first nucleic acid sequence encoding a first spacer sequence or a crRNA transcribed therefrom, wherein the first spacer sequence is complementary to a target nucleotide sequence from a target gene in a target bacterium. In some embodiments, the bacteriophage comprises a first nucleic acid sequence encoding a first spacer sequence or a crRNA transcribed therefrom, wherein the first spacer sequence is complementary to a target nucleotide sequence from a target gene in a target bacterium, provided that the bacteriophage is rendered lytic. In some embodiments, the bacteriophage is a temperate bacteriophage. In some embodiments, the bacteriophage is rendered lytic by removal, replacement, or inactivation of a lysogenic gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the bacteriophage is rendered lytic by the removal of a regulatory element of a lysogeny gene. In some embodiments, the bacteriophage is rendered lytic by the removal of a promoter of a lysogeny gene. In some embodiments, the bacteriophage is rendered lytic by the removal of a functional element of a lysogeny gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, the bacteriophage is rendered lytic via a second CRISPR array comprising a second spacer directed to a lysogenic gene. In some embodiments, the bacteriophage is rendered lytic by the insertion of one or more lytic genes. In some embodiments, the bacteriophage is rendered lytic by the insertion of one or more genes that contribute to the induction of a lytic cycle. In some embodiments, the bacteriophage is rendered lytic by altering the expression of one or more genes that contribute to the induction of a lytic cycle. In some embodiments, the bacteriophage phenotypically changes from a lysogenic bacteriophage to a lytic bacteriophage. In some embodiments, the phenotypic change is via a self-targeting CRISPR-Cas system to render a bacteriophage lytic since it is incapable of lysogeny. In some embodiments, the self-targeting CRISPR-Cas comprises a self-targeting crRNA from the prophage genome and kills lysogens. In some embodiments, the bacteriophage is rendered lytic by environmental alterations. In some embodiments, environmental alterations include, but are not limited to, alterations in temperature, pH, or nutrients; exposure to antibiotics, hydrogen peroxide, foreign DNA, or DNA damaging agents; presence of organic carbon, presence of heavy metal (e.g. in the form of chromium (VI)), or any combination thereof. In some embodiments, the bacteriophage that is rendered lytic is prevented from reverting to lysogenic state. In some embodiments, the bacteriophage that is rendered lytic is prevented from reverting back to lysogenic state by way of introducing an additional CRISPR array. In some embodiments, the bacteriophage does not confer any new properties onto the target bacterium beyond cellular death caused by lytic activity of the bacteriophage and/or the activity of the CRISPR array. Further disclosed herein, in some embodiments, are temperate bacteriophages comprising a first nucleic acid sequence encoding a first spacer sequence or a crRNA transcribed therefrom, wherein the first spacer sequence is complementary to a target nucleotide sequence from a target gene in a target bacterium, provided the bacteriophage is rendered lytic. In some embodiments, the bacteriophage infects multiple bacterial strains. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the target bacterium. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, glnS, dnaE, rpoA, rpoB, pheT, infB, rpsC, rplF, alaS, leuS, serS, rplD, gyrA, or metK. In some embodiments, the target nucleotide sequence is in a non-essential gene. In some embodiments, the target nucleotide sequence comprises at a portion of a gene that is unique to the first microbe over the second microbe, wherein the first microbe is either (i) a different genus (ii) a different species (iii) a different subspecies from the second microbe; or (iv) is in any way distinct from the second microbe by a functional parameter. In some embodiments, the functional parameter is a functional characteristic that relates to a functional outcome associated with, e.g., causing a disease, a syndrome, a condition, or a cause for discomfort to a subject, wherein the subject is a human or a non-human organism. In some embodiments, the subject is human. In some embodiments, the first microbe is associated with the disease, syndrome, condition, or cause for discomfort to the subject, whereas the second microbe is not. In some embodiments, the first microbe comprises a gene or a gene sequence that is associated with increased virulence or pathogenicity, that is absent in the second microbe. In some embodiments, the target nucleotide sequence comprising at a gene sequence that is unique to the first microbe with respect to the second microbe. In some embodiments, the first microbe comprises a gene or portion thereof that is associated with increased virulence or pathogenicity, that is absent in the second microbe. In some embodiments, the target nucleotide sequence comprising at a gene sequence that is unique to the first microbe with respect to the second microbe is a gene sequence that is associated with increased virulence or pathogenicity. In some embodiments, the target nucleotide sequence comprising at a gene sequence that is unique to the first microbe with respect to the second microbe is a gene sequence associated with the multiplication, growth, or survival of the first microbe. In some embodiments, the target nucleotide sequence comprising at a gene sequence that is unique to the first microbe with respect to the second microbe is a gene sequence that influences multiplication, growth, or survival of the first microbe, and does not influence the multiplication, growth, or survival of the second microbe. In some embodiments, the target nucleotide sequence comprising at a gene sequence that is unique to the first microbe with respect to the second microbe is a gene sequence that influences multiplication, growth, or survival of the first microbe with a higher efficiency than that of the second microbe. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a target bacterium. In some embodiments, the spacer sequence is complementary to a target nucleotide sequence of a sequence present in the target bacterium. In some embodiments, the spacer sequence is complementary to a target nucleotide sequence that comprises all or a part of a promoter sequence of the essential gene. In some embodiments, the first nucleic acid sequence comprises a first CRISPR array comprising at least one repeat sequence. In some embodiments, the at least one repeat sequence is operably linked to the first spacer sequence at either its 5′ end, or its 3′ end. In some embodiments, the target bacterium is C. difficile.

In some embodiments, the bacteriophage orphagemid DNA is from a lysogenic or temperate bacteriophage. In some embodiments, the bacteriophages or phagemids include but are not limited to P1 phage, a M13 phage, a λ phage, a T4 phage, a T7 phage, a T7m phage, a φC2 phage, a φCD27 phage, a φNM1 phage, Bc431 v3 phage, 410 phage, φ25 phage, 4φ151 phage, A511-like phages, B054, 0176-like phages, or Campylobacter phages (such as NCTC 12676 and NCTC 12677). In some embodiments, the bacteriophage is $CD146 C. difficile bacteriophage. In some embodiments, the bacteriophage is $CD24-2 C. difficile bacteriophage.

In some embodiments, a plurality of bacteriophages are used together. In some embodiments, the plurality of bacteriophages used together targets the same or different bacteria within a sample or subject. In some embodiments, the plurality of bacteriophages used together comprises T4 phage, T7 phage, T7m phage, or any combination of bacteriophages described herein.

In some embodiments, bacteriophages of interest are obtained from environmental sources, or commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

In some embodiments, the nucleic acid is inserted into the bacteriophage genome. In some embodiments, the nucleic acid comprises a crArray, a Cas system, or a combination thereof. In some embodiments, the nucleic acid is inserted into the bacteriophage genome at a transcription terminator site at the end of an operon of interest. In some embodiments, the nucleic acid is inserted into the bacteriophage genome as a replacement for one or more removed non-essential genes. In some embodiments, the nucleic acid is inserted into the bacteriophage genome as a replacement for one or more removed lysogenic genes. In some embodiments, the replacement of non-essential and/or lysogenic genes with the nucleic acid enhances the lytic activity of the bacteriophage. In some embodiments, the replacement of non-essential and/or lysogenic genes with the nucleic acid renders a lysogenic bacteriophage lytic.

In some embodiments, the nucleic acid is introduced into the bacteriophage genome at a first location while one or more non-essential and/or lysogenic genes are separately removed and/or inactivated from the bacteriophage genome at a separate location. In some embodiments, the removal of one or more non-essential and/or lysogenic genes renders a lysogenic bacteriophage into a lytic bacteriophage. Similarly, in some embodiments, one or more lytic genes are introduced into the bacteriophage so as to render a non-lytic, lysogenic bacteriophage into a lytic bacteriophage.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

In some embodiments, the bacteriophage is φCD146 C. difficile bacteriophage. In some embodiments, the bacteriophage is φCD24-2 C. difficile bacteriophage.

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle. In some embodiments, the non-essential gene to be removed and/or replaced from the bacteriophage is gp49 from φCD146 C. difficile bacteriophage. In some embodiments, the non-essential gene to be removed and/or replaced from the bacteriophage is gp75 from φCD24-2 C. difficile bacteriophage.

Disclosed herein, in certain embodiments, are bacteriophages comprising a complete exogenous CRISPR-Cas system. In some embodiments, the CRISPR-Cas system is Type I CRISPR-Cas system, Type II CRISPR-Cas system, Type III CRISPR-Cas system, Type IV CRISPR-Cas system, Type V CRISPR-Cas system, or Type VI CRISPR-Cas system. Disclosed herein, in certain embodiments, are bacteriophages comprising a nucleic acid sequence encoding a Type I CRISPR-Cas system comprising: (a) a CRISPR array; (b) a Cascade polypeptide; and (c) a Cas3 polypeptide.

### CRISPR Array

In some embodiments, the CRISPR array (crArray) comprises a spacer sequence and at least one repeat sequence. In some embodiments, the CRISPR array encodes a processed, mature crRNA. In some embodiments, the mature crRNA is introduced into a phage or a target bacterium. In some embodiments, an endogenous or exogenous Cas6 processes the CRISPR array into mature crRNA. In some embodiments, an exogenous Cas6 is introduced into the phage. In some embodiments, the phage comprises an exogenous Cas6. In some embodiments, an exogenous Cas6 is introduced into a target bacterium.

In some embodiments, the CRISPR array comprises a spacer sequence. In some embodiments, the CRISPR array further comprises at least one repeat sequence. In some embodiments, the at least one repeat sequence is operably linked to the spacer sequence at either its 5′ end or its 3′ end. In some embodiments, the CRISPR array is of any length, and comprises any number of spacer nucleotide sequences alternating with repeat nucleotide sequences necessary to achieve the desired level of killing of a target bacterium by targeting one or more essential genes. In some embodiments, the CRISPR array comprises, consists essentially of, or consists of 1 to about 100 spacer nucleotide sequences, each linked on its 5′ end and its 3′ end to a repeat nucleotide sequence. In some embodiments, the CRISPR array comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

### Spacer Sequence

In some embodiments, the spacer sequence is complementary to a target nucleotide sequence in a target bacterium. In some embodiments, the target nucleotide sequence is a coding region. In some embodiments, the coding region is an essential gene. In some embodiments, the coding region is a nonessential gene. In some embodiments, the target nucleotide sequence is a noncoding sequence. In some embodiments, the noncoding sequence is an intergenic sequence. In some embodiments, the spacer sequence is complementary to a target nucleotide sequence of a highly conserved sequence in a target bacterium. In some embodiments, the spacer sequence is complementary to a target nucleotide sequence of a sequence present in a first microbial population and not present in a second microbial population in the target bacterium. In some embodiments, the spacer sequence is complementary to a target nucleotide sequence that comprises all or a part of a promoter sequence of the essential gene. In some embodiments, the spacer sequence is complementary to a target nucleotide sequence of a sequence present in a first microbial population, and not present in a second microbial population. In some embodiments, the spacer sequence comprises one, two, three, four, or five mismatches as compared to the target nucleotide sequence. In some embodiments, the mismatches are contiguous. In some embodiments, the mismatches are noncontiguous. In some embodiments, the spacer sequence has 70% complementarity to a target nucleotide sequence. In some embodiments, the spacer sequence has 80% complementarity to a target nucleotide sequence. In some embodiments, the spacer sequence is 85%, 90%, 95%, 96%, 97%, 98%, 99%, or 100% complementarity to a target nucleotide sequence. In some embodiments, the spacer sequence has 100% complementarity to the target nucleotide sequence. In some embodiments, the spacer sequence has complete complementarity or substantial complementarity over a region of a target nucleotide sequence that are at least about 8 nucleotides to about 150 nucleotides in length. In some embodiments, a spacer sequence has complete complementarity or substantial complementarity over a region of a target nucleotide sequence that is at least about 20 nucleotides to about 100 nucleotides in length. In some embodiments, the 5′ region of the spacer sequence is 100% complementary to a target nucleotide sequence while the 3′ region of the spacer is substantially complementary to the target nucleotide sequence and therefore the overall complementarity of the spacer sequence to the target nucleotide sequence is less than 100%. For example, in some embodiments, the first 7, 8, 9, 10, 11, 12, 13, 14, 15, or 16 nucleotides in the 3′ region of a 20 nucleotide spacer sequence (seed region) are 100% complementary to the target nucleotide sequence, while the remaining nucleotides in the 5′ region of the spacer sequence are substantially complementary (e.g., at least about 70% complementary) to the target nucleotide sequence. In some embodiments, the first 7 to 12 nucleotides of the 3′ end of the spacer sequence are 100% complementary to the target nucleotide sequence, while the remaining nucleotides in the 5′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleotide sequence. In some embodiments, the first 7 to 10 nucleotides in the 3′ end of the spacer sequence are 75%-99% complementary to the target nucleotide sequence, while the remaining nucleotides in the 5′ region of the spacer sequence are at least about 50% to about 99% complementary to the target nucleotide sequence. In some embodiments, the first 7 to 10 nucleotides in the 3′ end of the spacer sequence are 100% complementary to the target nucleotide sequence, while the remaining nucleotides in the 5′ region of the spacer sequence are substantially complementary (e.g., at least about 70% complementary) to the target nucleotide sequence. In some embodiments, the first 10 nucleotides (within the seed region) of the spacer sequence are 100% complementary to the target nucleotide sequence, while the remaining nucleotides in the 5′ region of the spacer sequence are substantially complementary (e.g., at least about 70% complementary) to the target nucleotide sequence. In some embodiments, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) have about 75% complementarity or more (75% to about 100% complementarity) to the target nucleotide sequence, while the remainder of the spacer sequence has about 50% or more complementarity to the target nucleotide sequence. In some embodiments, the first 8 nucleotides at the 5′ end of the spacer sequence have 100% complementarity to the target nucleotide sequence, or have one or two mutations, and therefore the spacer sequence is about 88% complementary or about 75% complementary to the target nucleotide sequence, respectively, while the remainder of the spacer nucleotide sequence is at least about 50% or more complementary to the target nucleotide sequence.

In some embodiments, the spacer sequence is about 15 nucleotides to about 150 nucleotides in length. In some embodiments, the spacer nucleotide sequence is about 15 nucleotides to about 100 nucleotides in length (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides, or more). In some embodiments, the spacer nucleotide sequence is a length of about 8 to about 150 nucleotides, about 8 to about 100 nucleotides, about 8 to about 50 nucleotides, about 8 to about 40 nucleotides, about 8 to about 30 nucleotides, about 8 to about 25 nucleotides, about 8 to about 20 nucleotides, about 10 to about 150 nucleotides, about 10 to about 100 nucleotides, about 10 to about 80 nucleotides, about 10 to about 50 nucleotides, about 10 to about 40, about 10 to about 30, about 10 to about 25, about 10 to about 20, about 15 to about 150, about 15 to about 100, about 15 to about 50, about 15 to about 40, about 15 to about 30, about 20 to about 150 nucleotides, about 20 to about 100 nucleotides, about 20 to about 80 nucleotides, about 20 to about 50 nucleotides, about 20 to about 40, about 20 to about 30, about 20 to about 25, at least about 8, at least about 10, at least about 15, at least about 20, at least about 25, at least about 30, at least about 32, at least about 35, at least about 40, at least about 44, at least about 50, at least about 60, at least about 70, at least about 80, at least about 90, at least about 100, at least about 110, at least about 120, at least about 130, at least about 140, at least about 150 nucleotides in length, or more, and any value or range therein. In some embodiments, the P. aeruginosa Type I-C Cas system has a spacer length of about 30 to 39 nucleotides, about 31 to about 38 nucleotides, about 32 to about 37 nucleotides, about 33 to about 36 nucleotides, about 34 to about 35 nucleotides, or about 35 nucleotides. In some embodiments, the P. aeruginosa Type I-C Cas system has a spacer length of about 34 nucleotides. In some embodiments, the P. aeruginosa Type I-C Cas system has a spacer length of at least about 10, at least about 15, at least about 20, at least about 21, at least about 22, at least about 23, at least about 24, at least about 25, at least about 26, at least about 27, at least about 29, at least about 29, at least about 30, at least about 31, at least about 32, at least about 33, at least about 34, at least about, at least about 35, at least about 36, at least about 37, at least about 38, at least about 39, at least about 20, at least about 41, at least about 42, at least about 43, at least about 44, at least about 45, or more than about 45 nucleotides.

Non-limiting example spacer sequences are provided in Table 3. In some cases, a spacer sequence is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 37-48. In some cases, the spacer sequence comprises any one of SEQ ID NOS: 37-48.

In some embodiments, a spacer sequence is substantially complementary to a target sequence in an adherent-invasive E. coli (AIEC). As a non-limiting example, the spacer sequence is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 37-39. For instance, the spacer sequence is SEQ ID NO: 37, 38, or 39. In some cases, a Type I CRISPR-Cas array is provided comprising a spacer sequence at least 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 37. This array may be used with a Type IF CRISPR-Cas system, such as ECIF. In some cases, a Type I CRISPR-Cas array is provided comprising a spacer sequence at least 80%, 81%, 82%, 83%, 84%, 85%, 86%, 87%, 88%, 89%, 90, 91%, 92%, 93%, 94%, 95%, 96%, 97%, 98%, or 99% identical SEQ ID NO: 38. This array may be used with a Type IC CRISPR-Cas system, such as PAIC. In some cases, a Type I CRISPR-Cas array is provided comprising a spacer sequence at least 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 39. This array may be used with a Type IE CRISPR-Cas system, such as ECIE.

In some embodiments, a spacer sequence is substantially complementary to a target sequence in a pks+E. coli (PKS+). As a non-limiting example, the spacer sequence is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 40-42. For instance, the spacer sequence is SEQ ID NO: 40, 41 or 42. In some cases, a Type I CRISPR-Cas array is provided comprising a spacer sequence at least 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 40. This array may be used with a Type IF CRISPR-Cas system, such as ECIF. In some cases, a Type I CRISPR-Cas array is provided comprising a spacer sequence at least 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 41. This array may be used with a Type IC CRISPR-Cas system, such as PAIC. In some cases, a Type I CRISPR-Cas array is provided comprising a spacer sequence at least 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 42. This array may be used with a Type IE CRISPR-Cas system, such as ECIE.

In some embodiments, a spacer sequence is substantially complementary to Bacteroides fragilis toxin (BFT). As a non-limiting example, the spacer sequence is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 43-45. For instance, the spacer sequence is SEQ ID NO: 43, 44, or 45. In some cases, a Type I CRISPR-Cas array is provided comprising a spacer sequence at least 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 43. This array may be used with a Type IF CRISPR-Cas system, such as ECIF. In some cases, a Type I CRISPR-Cas array is provided comprising a spacer sequence at least 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 44. This array may be used with a Type IC CRISPR-Cas system, such as PAIC. In some cases, a Type I CRISPR-Cas array is provided comprising a spacer sequence at least 80% 81% 82%, 83%, 84%, 85%, 86%, 87%, 88%, 89%, 90%, 91%, 92%, 93%, 94%, 95%, 96%, 97%, 98%, or 99% identical SEQ ID NO: 45. This array may be used with a Type IE CRISPR-Cas system, such as ECIE.

In some embodiments, a spacer sequence is substantially complementary to vancomycin-resistant Enterococcus sp. As a non-limiting example, the spacer sequence is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOS: 46-48. For instance, the spacer sequence is SEQ ID NO: 46, 47, or 48. In some cases, a Type I CRISPR-Cas array is provided comprising a spacer sequence at least 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 46. This array may be used with a Type IF CRISPR-Cas system, such as ECIF. In some cases, a Type I CRISPR-Cas array is provided comprising a spacer sequence at least 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 47. This array may be used with a Type IC CRISPR-Cas system, such as PAIC. In some cases, a Type I CRISPR-Cas array is provided comprising a spacer sequence at least 80%, 81%, 82%, 83%, 84%, 85%, 86%, 87%, 88%, 89%, 90%, 91%, 92%, 93%, 94%, 95%, 96%, 97%, 98%, or 99% identical SEQ ID NO: 48. This array may be used with a Type IE CRISPR-Cas system, such as ECIE.

In some embodiments, a CRISPR-Cas system comprises a spacer sequence targeting an AIEC gene from an E. coli invasion gene.

In some embodiments, a CRISPR-Cas system comprises a spacer sequence targeting an PKS+E. coli gene, e.g., Colibactin.

In some embodiments, a CRISPR-Cas system comprises a spacer sequence targeting an Enterotoxigenic B. fragilis gene, e.g., B. fragilis toxin.

In some embodiments, a CRISPR-Cas system comprises a spacer sequence targeting a Vancomycin-resistant Enterococcus sp gene, e.g., Vancomycin resistance genes

Some exemplary genes and sequences targeted are provided below:

AIEC
ECIF -
(SEQ ID NO: 37)
ATATTACGGATTGTCGCATAGGCGAAATTAT
(ibeA) invasion protein IbeA [Ibes (VF0237)][Escherichia coli
O18:K1:H7 str. RS218]
PAIC -
(SEQ ID NO: 38)
TCCTCCATCCATTTTTTTTTAATACGTGGAATGT
(neuE) polysialic acid biosynthesis protein [K1 capsule (VF0239)]
[Escherichia coli O45:K1:H7 str. S88]
ECIE -
(SEQ ID NO: 39)
TTTCTGAATTACTCTAATCAAAAAACGTGCAT
(neuE) polysialic acid biosynthesis protein [K1 capsule (VF0239)]
[Escherichia coli O45:K1:H7 str. S88]
PKS+
ECIF -
(SEQ ID NO: 40)
TATATCATTTTCACTTCCGGCTCCACCGGTA
(clbK) colibactin hybrid non-ribosomal peptide synthetase/type I
polyketide synthase ClbK [Colibactin (VF0573)]
PAIC -
(SEQ ID NO: 41)
GCGCGCTGGATGGTCTGGGCATTGCACTGACGGC
(clbK) colibactin hybrid non-ribosomal peptide synthetase/type I
polyketide synthase ClbK [Colibactin (VF0573)]
ECIE -
(SEQ ID NO: 42)
CTGGACACGCCTGCTGCGCCGTTGCCTGAAGT
(clbK) colibactin hybrid non-ribosomal peptide synthetase/type I
polyketide synthase ClbK [Colibactin (VF0573)]
BFT
ECIF -
(SEQ ID NO: 43)
AGTTATGCATCGTGCATATGGTGCGCAACAG
(astA) heat-stable enterotoxin 1 [EAST1 (VF0216)]
PAIC -
(SEQ ID NO: 44)
CTCTAGCTGTATCCTGGTGCTGAATCACTTTCCC
(nheB) non-hemolytic enterotoxin B [Nhe (VF0533)]
ECIE -
(SEQ ID NO: 45)
TTCGATTAATAGTGAGTTTAAAGGAAATATGA
(nheC) non-hemolytic enterotoxin C [Nhe (VF0533)]
Vancomycin-resistant Enterococcus
ECIF -
(SEQ ID NO: 46)
GGTGATAAAGCACGAACCCATAAAGAGGATA
VanM-type vancomycin resistance histidine kinase VanS
[organism = Enterococcus faecium][GeneID = 66455786]
PAIC -
(SEQ ID NO: 47)
GTGCTTTATCACCGGTATAGAACATATCAAAAAG
VanM-type vancomycin resistance histidine kinase VanS
[organism = Enterococcus faecium][GeneID = 66455786]
ECIE -
(SEQ ID NO: 48)
GGTTTGTTGATGAAGAAGAAGTGGTTGTTCAA
VanM-type vancomycin resistance histidine kinase VanS
[organism = Enterococcus faecium][GeneID = 66455786]

TABLE 3
Example spacer sequences targeting adherent-invasive E. coli (AIEC), PKS+
E.coli (PKS+), Bacteroides fragilis toxin (BFT) and Enterococcus sp.

| | SEQ | |
|---|---|---|
| | ID | |
| | NO | |
| | | Adherent-invasive E. coli (AIEC) |
| Type | 37 | ATATTACGGATTGTCGCATAGGCGAAATTAT |
| 1F | | |
| (ECIF) | | |
| Type | 38 | TCCTCCATCCATTTTTTTTTAATACGTGGAATGT |
| (PAIC) | | |
| Type | 39 | TTTCTGAATTACTCTAATCAAAAAACGTGCAT |
| 1E | | |
| (ECIE) | | |
| | | PKS+ E.coli (PKS+) |
| Type | 40 | TATATCATTTTCACTTCCGGCTCCACCGGTA |
| 1F | | |
| (ECIF) | | |
| Type | 41 | GCGCGCTGGATGGTCTGGGCATTGCACTGACGGC |
| (PAIC) | | |
| Type | 42 | CTGGACACGCCTGCTGCGCCGTTGCCTGAAGT |
| 1E | | |
| (ECIE) | | |
| | | Enterotoxigenic B. fragilis |
| Type | 43 | AGTTATGCATCGTGCATATGGTGCGCAACAG |
| 1F | | |
| (ECIF) | | |
| Type | 44 | CTCTAGCTGTATCCTGGTGCTGAATCACTTTCCC |
| (PAIC) | | |
| Type | 45 | TTCGATTAATAGTGAGTTTAAAGGAAATATGA |
| 1E | | |
| (ECIE) | | |
| | | Vancomycin-resistant Enterococcus sp |
| Type | 46 | GGTGATAAAGCACGAACCCATAAAGAGGATA |
| 1F | | |
| (ECIF) | | |
| Type | 47 | GTGCTTTATCACCGGTATAGAACATATCAAAAAG |
| (PAIC) | | |
| Type | 48 | GGTTTGTTGATGAAGAAGAAGTGGTTGTTCAA |
| 1E | | |
| (ECIE) | | |

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The term “homology” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

In some embodiments, the identity of two or more spacer sequences of the CRISPR array is the same. In some embodiments, the identity of two or more spacer sequences of the CRISPR array is different. In some embodiments, the identity of two or more spacer sequences of the CRISPR array is different, but are complementary to one or more target nucleotide sequences. In some embodiments, the identity of two or more spacer nucleotide sequences of the CRISPR array is different, and are complementary to one or more target nucleotide sequences that are overlapping sequences. In some embodiments, the identity of two or more spacer nucleotide sequences of the CRISPR array is different, and are complementary to one or more target nucleotide sequences that are not overlapping sequences. In some embodiments, the target nucleotide sequence is about 10 to about 40 consecutive nucleotides in length located immediately adjacent to a PAM sequence (PAM sequence located immediately 3′ of the target region) in the genome of the organism. In some embodiments, a target nucleotide sequence is located adjacent to, or flanked by, a PAM (protospacer adjacent motif).

In some embodiments, the target nucleotide sequence in the bacterium to be killed is any essential target nucleotide sequence of interest. In some embodiments, the target nucleotide sequence is a non-essential sequence. In some embodiments, a target nucleotide sequence comprises, consists essentially of, or consist of, all or a part of a nucleotide sequence encoding a promoter, or a complement thereof, of the essential gene. In some embodiments, the spacer nucleotide sequence is complementary to a promoter, or a part thereof, of the essential gene. In some embodiments, the target nucleotide sequence comprises all, or a part of, a nucleotide sequence located on a coding or a non-coding strand of the essential gene. In some embodiments, the target nucleotide sequence comprises all, or a part of, a nucleotide sequence located on a coding of a transcribed region of the essential gene.

In some embodiments, the essential gene is any gene of an organism that is critical for its survival. However, being essential is highly dependent on the circumstances in which an organism lives. For instance, a gene required to digest starch is only essential if starch is the only source of energy. In some embodiments, the target nucleotide sequence comprises all or a part of a promoter sequence for the target gene. In some embodiments, the target nucleotide sequence comprises all, or a part of, a nucleotide sequence located on a coding strand of a transcribed region of the target gene. In some embodiments, the target nucleotide sequence comprises at least a portion of an essential gene that is needed for survival of the target bacterium. In some embodiments, the essential gene is Tsf, acpP, gapA, infA, secY, csrA, trmD, ftsA, fusA, glyQ, eno, nusG, dnaA, dnaS, pheS, rplB, gltX, hisS, rplC, aspS, gyrB, ginS, dnaE, rpoA, rpoB, pheT, infB, rpsC, rplF, alaS, leuS, serS, rplD, gyrA, or metK. In some embodiments, the essential genes may be shared between the first microbe and the second microbe and therefore the antimicrobial agent does not distinguish between the two. In some embodiments, a gene sequence is targeted in the first microbe that is not present in the second microbe, wherein the gene sequence referred to may contain a mutation, i.e., a mutated gene sequence. In some embodiments, the first microbe may comprise a mutation with respect to a wild-type sequence. In some embodiments, the second microbe may comprise a mutation with respect to a wild-type sequence. In some embodiments, the first microbe comprises a gene or portion thereof that is associated with increased virulence or pathogenicity to the subject, that is absent in the second microbe. In some embodiments, the target nucleotide sequence comprising at a gene sequence that is unique to the first microbe with respect to the second microbe is a gene sequence that is associated with increased virulence or pathogenicity. In some embodiments, the target nucleotide sequence comprising at a gene sequence that is unique to the first microbe with respect to the second microbe is a gene sequence associated with the multiplication, growth or survival of the first microbe. In some embodiments, the target nucleotide sequence comprising at a gene sequence that is unique to the first microbe with respect to the second microbe is a gene sequence that influences multiplication, growth or survival of the first microbe, and does not influence the multiplication, growth or survival of the second microbe. In some embodiments, the target nucleotide sequence comprising at a gene sequence that is unique to the first microbe with respect to the second microbe is a gene sequence that influences multiplication, growth or survival of the first microbe with a higher efficiency than that of the second microbe. In some embodiments the essential genes are not targeted by the antimicrobial agent. In some embodiments, a non-essential gene is any gene of an organism that is not critical for survival. However, being non-essential is highly dependent on the circumstances in which an organism lives.

In some embodiments, the target nucleic acid sequence is an AIEC-specific sequence. In some embodiments, the target nucleic acid sequence is a pks+DNA sequence.

In some embodiments, the target nucleic acid sequence is an AIEC-specific sequence. In some embodiments, the target nucleic acid sequence is a pks+DNA sequence, such as a pks genomic island, or a gene that encodes colibactin. In some embodiments, the target nucleic acid sequence is a sequence encoding an enterotoxin. In some embodiments, the target nucleic acid sequence is a sequence encoding BFT or fragilysin. In some embodiments other BFTs may be considered. There may be 11 other toxins, and genes encodings such toxins in B. fragilis. In some embodiments, the target nucleic acid sequence is a toxin sequence. In some embodiments, the toxin sequence is a shiga toxin. In some embodiments, the toxin sequence is a verocytotoxin sequence. In some embodiments, the target nucleic acid sequence is a sequence and is present in a Shigella spp., and not in a commensal E. coli species. In some embodiments, the target nucleic acid sequence is present in E. faecalis or E. faecium, and not in a commensal Enterococcus spp.

In some embodiments, the target nucleic acid sequence is present in an EPEC, and not in a commensal E. coli. In some embodiments, the target nucleic acid sequence is present in a B. fragilis or B. thetaiotamicron, and not in a commensal strain of E. coli. In some embodiments, the subject may have acne vulgaris. In some embodiments, the target nucleic acid sequence is present in a C. acnes that contributes to acne or skin inflammation in a subject, and not in a commensal strain of C. acnes. In some embodiments, the genomic sequence associated with the microbial species found in the microbiome of a formula-fed infant is present in a first microbial population, and not in a microbial species found in the gut microbiome of a breast-fed infant. In some embodiments, the target nucleic acid sequence is present in a Campylobacter jejuni strain associated with anxiety, and not in a commensal strain of Campylobacter jejuni. In some embodiments, the target nucleic acid sequence is present in a microbial species associated with elevated levels of IgM and IgA in a subject, and not in a commensal strain of the microbial species. In some embodiments, the target nucleic acid sequence is present in a Mycobacterium avium subspecies paratuberculosis (MAP), and not in a commensal strain.

In some embodiments, non-limiting examples of the target nucleotide sequence of interest include a target nucleotide sequence encoding a transcriptional regulator, a translational regulator, a polymerase gene, a metabolic enzyme, a transporter, an RNase, a protease, a DNA replication enzyme, a DNA modifying or degrading enzyme, a regulatory RNA, a transfer RNA, or a ribosomal RNA. In some embodiments, the target nucleotide sequence is from a gene involved in cell-division, cell structure, metabolism, motility, pathogenicity, virulence, or antibiotic resistance. In some embodiments, the target nucleotide sequence is from a hypothetical gene whose function is not yet characterized. Thus, for example, these genes are any genes from any bacterium.

The appropriate spacer sequences for a full-construct phage may be identified by locating a search set of representative genomes, searching the genomes with relevant parameters, and determining the quality of a spacer for use in a CRISPR engineered phage.

First, a suitable search set of representative genomes is located and acquired for the organism/species/target of interest. The set of representative genomes may be found in a variety of databases, including without limitations the NCBI genbank or the PATRIC database. NCBI genbank is one of the largest databases available, and contains a mixture of reference and submitted genomes for nearly every organism sequenced to date. Specifically, for pathogenic prokaryotes, the PATRIC (Pathosystems Resource Integration Center) database provides an additional comprehensive resource of genomes, and provides a focus on clinically relevant strains and genomes relevant to a drug product. Both of the above databases allow for bulk downloading of genomes via FTP (File Transfer Protocol) servers, enabling rapid and programmatic dataset acquisition.

Next, the genomes are searched with relevant parameters to locate suitable spacer sequences. Genomes may be read from start to end, in both the forward and reverse complement orientations, to locate contiguous stretches of DNA that contain a PAM (Protospacer Adjacent Motif) site. The spacer sequence will be the N-length DNA sequence 3′ or 5′ adjacent to the PAM site (depending on the CRISPR system type), where N is specific to the Cas system of interest and is generally known ahead of time. Characterizing the PAM sequence and spacer sequences maybe performed during the discovery and initial research of a Cas system. Every observed PAM-adjacent spacer may be saved to a file and/or database for downstream use. The exact PAM sequence that is required varies between each different CRISPR-Cas system and is identified through established bioinformatics and experimental procedures.

Next, the quality of a spacer for use in a CRISPR engineered phage is determined.

Each observed spacer may be evaluated to determine how many of the evaluated genomes they are present in. The observed spacers may be evaluated to see how many times they may occur in each given genome. Spacers that occur in more than one location per genome may be advantageous because the Cas system may not be able to recognize the target site if a mutation occurs, and each additional “backup” site increases the likelihood that a suitable, non-mutated target location will be present. The observed spacers may be evaluated to determine whether they occur in functionally annotated regions of the genome. If such information is available, the functional annotations may be further evaluated to determine whether those regions of the genome are “essential” for the survival and function of the organism. By focusing on spacers that occur in all, or nearly all, evaluated genomes of interest (>=99%), the spacer selection may be broadly applicable to many targeted genomes. Provided a large selection pool of conserved spacers exists, preference may be given to spacers that occur in regions of the genome that have known function, with higher preference given if those genomic regions are “essential” for survival and occur more than 1 time per genome.

The spacer sequences for a full construct phage, in some embodiments, are validated. In some embodiments, a first step comprises identifying a plasmid that replicates in the organism, species, or target of interest. In some embodiments, the plasmid has a selectable marker. In some embodiments, the selectable marker is an antibiotic-resistance gene. In some embodiments, an expression cassette includes a nucleotide sequence for a selectable marker. In some embodiments, the selectable marker is adenine deaminase (ada), blasticidin S deaminases (Bsr, BSD), bleomycin-bindingprotein (Ble), Neomycin phosphotransferase (neo), histidinol dehydrogenase (hisD), glutamine synthetase (GS), dihydrofolate reductase (dhfr), cytosine deaminase (codA), puromycin N-acetyltransferase (Pac), hygromycin B phosphotransferase (Hph), ampicillin, chloramphenicol, kanamycin, tetracycline, polymyxin B, erythromycin, carbenicillin, streptomycin, spectinomycin, puromycin N-acetyltransferase (Pac), or zeocin (Sh bla). In some embodiments, the selectable marker is a gene involved in thymidylate synthase, thymidine kinase, dihydrofolate reductase, or glutamine synthetase. In some embodiments, the selectable marker is a gene encoding a fluorescent protein.

In some embodiments, a second step comprises inserting the genes encoding the Cas system into the plasmid such that they will be expressed in the organism, species, or target of interest. In some embodiments, a promoter is provided upstream of the Cas system. In some embodiments, the promoter is recognized by the organism, species, or target of interest to drive the expression of the Cas system. Exemplary promoters include, but are not limited to, L-arabinose inducible (araBAD, P_{BAD}) promoter, any lac promoter, L-rhamnose inducible (rhaPBAD) promoter, T7 RNA polymerase promoter, trc promoter, tac promoter, lambda phage promoter (p_{L}p_{L}-9G-50), anhydrotetracycline-inducible (tetA) promoter, trp, Ipp, phoA, recA, pro U, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, α-amylase (Pamy), Ptms, P43 (comprised of two overlapping RNA polymerase 6 factor recognition sites, 6A, GB), Ptms, P43, rplK-rplA, ferredoxin promoter, and/or xylose promoter. In some embodiments, the promoter is a BBa_J23102, BBa_J23104, or BBa_J23109. In some embodiments the promoter is derived from the organism, species, or target bacterium, such as endogenous CRISPR promoter, endogenous Cas operon promoter, p16, plpp, or ptat. In some embodiments, the promoter is a phage promoter, such as the promoter for gp105 or gp245. In some embodiments, a ribosomal binding site (RBS) is provided between the promoter and the Cas system. In some embodiments, the RBS is recognized by the organism, species, or target of interest.

In some embodiments, a third step comprises providing genome-targeting spacers into the plasmid. In some embodiments, the genome-targeting spacers are identified using bioinformatics. In some embodiments, the genome-targeting spacers are provided upstream of the repeat-spacer-repeat. In some embodiments, a promoter is provided. In some embodiments, the promoter is recognized by the organism, species, or target of interest to drive the expression of the crRNA. In some embodiments, the cloning for the third step comprises using an organism or species that is not targeted by the spacer being cloned.

In some embodiments, a fourth step comprises providing a non-target spacer into the plasmid that expresses the Cas system. In some embodiments, the non-target spacer comprises a sequence is random. In some embodiments, the non-target spacer comprises a sequence that does not comprise targeting sites in the genome of the organism, species, or target of interest. In some embodiments, the non-target spacer sequence is determined using bioinformatics to not comprise targeting sites in the genome of the organism, species, or target of interest. In some embodiments, the non-target spacer sequence is provided upstream of the repeat-spacer-repeat. In some embodiments, a promoter is provided. In some embodiments, the promoter is recognized by the organism, species, or target of interest to drive the expression of the crRNA.

In some embodiments, a fifth step comprises determining an efficacy of each spacer generated. In some embodiments, the killing efficacy is determined. In some embodiments, the efficacy of each spacer at targeting the bacterial genome is determined. In some embodiments, the plasmids comprising the spacer comprises about 0.5-fold, about 1-fold, 5-fold, 10-fold, 20-fold, 40-fold, 60-fold, 80-fold, or up to about 100 fold reduction in transfer rate as compared to a plasmid that comprises the non-targeting spacer.

### Repeat Nucleotide Sequences

In some embodiments, a repeat nucleotide sequence of the CRISPR array comprises a nucleotide sequence of any known repeat nucleotide sequence of a CRISPR-Cas system. In some embodiments, a repeat nucleotide sequence is of a synthetic sequence comprising the secondary structure of a native repeat from a CRISPR-Cas system (e.g., an internal hairpin). In some embodiments, the repeat nucleotide sequences are distinct from one another based on the known repeat nucleotide sequences of a CRISPR-Cas system. In some embodiments, the repeat nucleotide sequences are each composed of distinct secondary structures of a native repeat from a CRISPR-Cas system (e.g., an internal hairpin). In some embodiments, the repeat nucleotide sequences are a combination of distinct repeat nucleotide sequences operable with a CRISPR-Cas system.

In some embodiments, the spacer sequence is linked at its 5′ end to the 3′ end of a repeat sequence. In some embodiments, the spacer sequence is linked at its 5′ end to about 1 to about 8, about 1 to about 10, or about 1 to about 15 nucleotides of the 3′ end of a repeat sequence. In some embodiments, the about 1 to about 8, about 1 to about 10, about 1 to about 15 nucleotides of the repeat sequence are a portion of the 3′ end of a repeat sequence. In some embodiments, the spacer nucleotide sequence is linked at its 3′ end to the 5′ end of a repeat sequence. In some embodiments, the spacer is linked at its 3′ end to about 1 to about 8, about 1 to about 10, or about 1 to about 15 nucleotides of the 5′ end of a repeat sequence. In some embodiments, the about 1 to about 8, about 1 to about 10, about 1 to about 15 nucleotides of the repeat sequence are a portion of the 5′ end of a repeat sequence.

In some embodiments, the spacer nucleotide sequence is linked at its 5′ end to a first repeat sequence and linked at its 3′ end to a second repeat sequence to form a repeat-spacer-repeat sequence. In some embodiments, the spacer sequence is linked at its 5′ end to about 1 to about 8, about 1 to about 10, or about 1 to about 15 nucleotides of the 3′ end of a first repeat sequence and is linked at its 3′ end to about 1 to about 8, ab out 1 to about 10, or about 1 to about 15 nucleotides of the 5′ end of a second repeat sequence. In some embodiments, the about 1 to about 8, about 1 to about 10, about 1 to about 15 nucleotides of the first repeat sequence are a portion of the 3′ end of the first repeat sequence. In some embodiments, the about 1 to about 8, about 1 to about 10, about 1 to about 15 nucleotides of the second repeat sequence are a portion of the 3′ end of the second repeat sequence. In some embodiments, the spacer sequence is linked at its 5′ end to the 3′ end of a first repeat sequence and is linked at its 3′ end to the 5′ of a second repeat sequence where the spacer sequence and the second repeat sequence are repeated to form a repeat-(spacer-repeat)n sequence such that n is any integer from 1 to 100. In some embodiments, a repeat-(spacer-repeat)n sequence comprises, consists essentially of, or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

In some embodiments, the repeat sequence is identical to or substantially identical to a repeat sequence from a wild-type CRISPR Type I loci. In some embodiments, the repeat sequence comprises a portion of a wild type repeat sequence (e.g., 1, 2, 3, 4, 5, 6, 7, 8, 9, 10, 11, 12, 13, 14, 15 or more contiguous nucleotides of a wild type repeat sequence). In some embodiments, the repeat sequence comprises, consists essentially of, or consists of at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, or more nucleotides, or any range therein). In some embodiments, the repeat sequence comprises, consists essentially of, or consists of no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 nucleotides. In some embodiments, the repeat sequence comprises about 20 to 40, 21 to 40, 22 to 40 23 to 40, 24 to 40, 25 to 40, 26 to 40, 27 to 40, 28 to 40, 29 to 40, 30 to 30, 31 to 40, 32 to 40, 33 to 40, 34 to 40, 35 to 40, 36 to 40, 37 to 40, 38 to 40, 39 to 40, 20 to 39, 20 to 38, 20 to 37, 20 to 36, 20 to 35, 20 to 34, 20 to 33, 20 to 32, 20 to 31, 20 to 30, 20 to 29, 20 to 28, 20 to 26, 20 to 25, 20 to 24, 20 to 23, 20 to 22, or 20 to 21 nucleotides. In some embodiments, the repeat sequence comprises about 20 to 35, 21 to 35, 22 to 3523 to 35, 24 to 35, 25 to 35, 26 to 35, 27 to 35, 28 to 35, 29 to 35, 30 to 30, 31 to 35, 32 to 35, 33 to 35, 34 to 35, 25 to 40, 25 to 39, 25 to 38, 25 to 37, 25 to 36, 25 to 35, 25 to 34, 25 to 33, 25 to 32, 25 to 31, 25 to 30, 25 to 29, 25 to 28, 25 to 26 nucleotides. In some embodiments, the system is a P. aeruginosa Type I-C Cas system. In some embodiments, the P. aeruginosa Type I-C Cas system has a repeat length of about 25 to 38 nucleotides.

### Transcriptional Activators

In some embodiments, the nucleic acid sequence further comprises a transcriptional activator. In some embodiments, the transcriptional activator encoded regulates the expression of genes of interest within the target bacterium. In some embodiments, the transcriptional activator activates the expression of genes of interest within the target bacterium whether exogenous or endogenous. In some embodiments, the transcriptional activator activates the expression genes of interest within the target bacterium by disrupting the activity of one or more inhibitory elements within the target bacterium. In some embodiments, the inhibitory element comprises a transcriptional repressor. In some embodiments, the inhibitory element comprises a global transcriptional repressor. In some embodiments the inhibitory element is a histone-like nucleoid-structuring (H-NS) protein or homologue or functional fragment thereof. In some embodiments, the inhibitory element is a leucine responsive regulatory protein (LRP). In some embodiments, the inhibitory element is a CodY protein.

In some bacteria, the CRISPR-Cas system is poorly expressed and considered silent under most environmental conditions. In these bacteria, the regulation of the CRISPR-Cas system is the result of the activity of transcriptional regulators, for example histone-like nucleoid-structuring (H-NS) protein which is widely involved in transcriptional regulation of the host genome. H-NS exerts control over host transcriptional regulation by multimerization along AT-rich sites resulting in DNA bending. In some bacteria, such as E. coli, the regulation of the CRISPR-Cas3 operon is regulated by H-NS.

Similarly, in some bacteria, the repression of the CRISPR-Cas system is controlled by an inhibitory element, for example the leucine responsive regulatory protein (LRP). LRP has been implicated in binding to upstream and downstream regions of the transcriptional start sites. Notably, the activity of LRP in regulating expression of the CRISPR-Cas system varies from bacteria to bacteria. Unlike, H-NS which has broad inter-species repression activity, LRP has been shown to differentially regulate the expression of the host CRISPR-Cas system. As such, in some instances, LRP reflects a host-specific means of regulating CRISPR-Cas system expression in different bacteria.

In some instances, the repression of CRISPR-Cas system is also controlled by inhibitory element CodY. CodY is a GTP-sensing transcriptional repressor that acts through DNA binding. The intracellular concentration of GTP acts as an indicator for the environmental nutritional status. Under normal culture conditions, GTP is abundant and binds with CodY to repress transcriptional activity. However, as GTP concentrations decreases, CodY becomes less active in binding DNA, thereby allowing transcription of the formerly repressed genes to occur.

As such, CodY acts as a stringent global transcriptional repressor.

In some embodiments, the transcriptional activator is a LeuO polypeptide, any homolog or functional fragment thereof, a leuO coding sequence, or an agent that upregulates LeuO. In some embodiments, the transcriptional activator comprises any ortholog or functional equivalent of LeuO. In some bacteria, LeuO acts in opposition to H-NS by acting as a global transcriptional regulator that responds to environmental nutritional status of a bacterium. Under normal conditions, LeuO is poorly expressed. However, under amino acid starvation and/or reaching of the stationary phase in the bacterial life cycle, LeuO is upregulated. Increased expression of LeuO leads to it antagonizing H-NS at overlapping promoter regions to effect gene expression. Overexpression of LeuO upregulates the expression of the CRISPR-Cas system. In E coil and S. typhimurium, LeuO drives increased expression of the casABCDE operon which has predicted LeuO and H-NS binding sequences upstream of CasA.

In some embodiments, the expression of LeuO leads to disruption of an inhibitory element. In some embodiments, the disruption of an inhibitory element due to expression of LeuO removes the transcriptional repression of a CRISPR-Cas system. In some embodiments, the expression of LeuO removes transcriptional repression of a CRISPR-Cas system due to activity of H-NS. In some embodiments, the disruption of an inhibitory element due to the expression of LeuO causes an increase in the expression of a CRISPR-Cas system. In some embodiments, the increase in the expression of a CRISPR-Cas system due to the disruption of an inhibitory element caused by the expression of LeuO causes an increase in the CRISPR-Cas processing of a nucleic acid sequence comprising a CRISPR array. In some embodiments, the increase in the expression of a CRISPR-Cas system due to the disruption of an inhibitory element by the expression of LeuO causes an increase in the CRISPR-Cas processing of a nucleic acid sequence comprising a CRISPR array so as to increase the level of lethality of the CRISPR array against a bacterium. In some embodiments, transcriptional activator causes increase activity of a bacteriophage and/or the CRISPR-Cas system.

### Regulatory Elements

In some embodiments, the nucleic acid sequences disclosed herein are operatively associated with a variety of promoters, terminators and other regulatory elements for expression in various organisms or cells. In some embodiments, the nucleic acid sequence further comprises a leader sequence. In some embodiments, the nucleic acid sequence further comprises a promoter sequence. In some embodiments, at least one promoter and/or terminator is operably linked the CRISPR array. Any promoter useful with this disclosure is used and includes, for example, promoters functional with the organism of interest as well as constitutive, inducible, developmental regulated, tissue-specific/preferred-promoters, and the like, as disclosed herein. A regulatory element as used herein is endogenous or heterologous. In some embodiments, an endogenous regulatory element derived from the subject organism is inserted into a genetic context in which it does not naturally occur (e.g. a different position in the genome than as found in nature), thereby producing a recombinant or non-native nucleic acid.

In some embodiments, expression of the nucleic acid sequence is constitutive, inducible, temporally regulated, developmentally regulated, or chemically regulated. In some embodiments, the expression of the nucleic acid sequence is made constitutive, inducible, temporally regulated, developmentally regulated, or chemically regulated by operatively linking the nucleic acid sequence to a promoter functional in an organism of interest. In some embodiments, repression is made reversible by operatively linking the nucleic acid sequence to an inducible promoter that is functional in an organism of interest. The choice of promoter disclosed herein varies depending on the quantitative, temporal and spatial requirements for expression, and also depending on the host cell to be transformed.

Exemplary promoters for use with the methods, bacteriophages and compositions disclosed herein include promoters that are functional in bacteria. For example, L-arabinose inducible (araBAD, P_{BAD}) promoter, any lac promoter, L-rhamnose inducible (rhaPBAD) promoter, T7 RNA polymerase promoter, trc promoter, tac promoter, lambda phage promoter (p_{L}p_{L}-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, α-amylase (Pamy), Ptms, P43 (comprised of two overlapping RNA polymerase 6 factor recognition sites, (A, 6B), Ptms, P43, rplK-rplA, ferredoxin promoter, and/or xylose promoter. In some embodiments, the promoter is a BBa_J23102 promoter.

In some embodiments, the promoter works in abroad range of bacteria, such as BBa_J23104, BBa_J23109. In some embodiments the promoter is derived from the target bacterium, such as endogenous CRISPR promoter, endogenous Cas operon promoter, p16, plpp, or ptat. In some embodiments, the promoter is a phage promoter, such as the promoter for gp105 or gp245.

In some embodiments, inducible promoters are used. In some embodiments, chemical-regulated promoters are used to modulate the expression of a gene in an organism through the application of an exogenous chemical regulator. The use of chemically regulated promoters enables RNAs and/or the polypeptides encoded by the nucleic acid sequence to be synthesized only when, for example, an organism is treated with the inducing chemicals. In some embodiments where a chemical-inducible promoter is used, the application of a chemical induces gene expression. In some embodiments wherein a chemical-repressible promoter is used, the application of the chemical represses gene expression. In some embodiments, the promoter is a light-inducible promoter, where application of specific wavelengths of light induces gene expression. In some embodiments, a promoter is a light-repressible promoter, where application of specific wavelengths of light represses gene expression.

### Expression Cassette

In some embodiments, the nucleic acid sequence is an expression cassette or in an expression cassette. In some embodiments, the expression cassettes are designed to express the nucleic acid sequence disclosed herein. In some embodiments, the nucleic acid sequence is an expression cassette encoding components of a CRISPR-Cas system. In some embodiments, the nucleic acid sequence is an expression cassette encoding components of a Type I CRISPR-Cas system. In some embodiments, the nucleic acid sequence is an expression cassette encoding an operable CRISPR-Cas system. In some embodiments, the nucleic acid sequence is an expression cassette encoding the operable components of a Type I CRISPR-Cas system, including Cascade and Cas3. In some embodiments, the nucleic acid sequence is an expression cassette encoding the operable components of a Type I CRISPR-Cas system, including a crRNA, Cascade and Cas3.

In some embodiments, an expression cassette comprising a nucleic acid sequence of interest is chimeric, meaning that at least one of its components is heterologous with respect to at least one of its other components. In some embodiments, an expression cassette is naturally occurring but has been obtained in a recombinant form useful for heterologous expression.

In some embodiments, an expression cassette includes a transcriptional and/or translational termination region (i.e. termination region) that is functional in the selected host cell. In some embodiments, termination regions are responsible for the termination of transcription beyond the heterologous nucleic acid sequence of interest and for correct mRNA polyadenylation. In some embodiments, the termination region is native to the transcriptional initiation region, is native to the operably linked nucleic acid sequence of interest, is native to the host cell, or is derived from another source (i.e., foreign or heterologous to the promoter, to the nucleic acid sequence of interest, to the host, or any combination thereof). In some embodiments, terminators are operably linked to the nucleic acid sequence disclosed herein.

In some embodiments, an expression cassette includes a nucleotide sequence for a selectable marker. In some embodiments, the nucleotide sequence encodes either a selectable or a screenable marker, depending on whether the marker confers a trait that is selected f or by chemical means, such as by using a selective agent (e.g. an antibiotic), or on whether the marker is simply a trait that one identifies through observation or testing, such as by screening (e.g., fluorescence).

### Vectors

In some embodiments, the nucleic acid sequences disclosed herein (e.g. nucleic acid sequence comprising a CRISPR array and/or encoding a peptide or a biologically active fragment thereof) are used in connection with vectors. A vector comprises a nucleic acid molecule comprising the nucleotide sequence(s) to be transferred, delivered or introduced. Non-limiting examples of general classes of vectors include, but are not limited to, a viral vector, a plasmid vector, a phage vector, a phagemid vector, a cosmid vector, a fosmid vector, a bacteriophage, an artificial chromosome, or an agrobacterium binary vector in double or single stranded linear or circular form which may or may not be self-transmissible or mobilizable. In some embodiments, a vector transforms prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms. In some embodiments, a shuttle vector replicates in actinomycetes and bacteria and/or eukaryotes. In some embodiments, the nucleic acid in the vector are under the control of, and operably linked to, an appropriate promoter or other regulatory elements for transcription in a host cell. In some embodiments, the vector is a bi-functional expression vector which functions in multiple hosts.

### Codon Optimization

In some embodiments, the nucleic acid sequences disclosed herein are codon optimized for expression in any species of interest. Codon optimization involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. Codon optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original nucleotide sequence. In some embodiments, the nucleic acid sequences of this disclosure are codon optimized for expression in the organism/species of interest.

### Transformation

In some embodiments, the nucleic acid sequences, and/or expression cassettes disclosed herein are expressed transiently and/or stably incorporated into the genome of a host organism. In some embodiments, a the nucleic acid sequence and/or expression cassettes disclosed herein is introduced into a cell by any method known to those of skill in the art. Exemplary methods of transformation include transformation via electroporation of competent cells, passive uptake by competent cells, chemical transformation of competent cells, as well as any other electrical, chemical, physical (mechanical) and/or biological mechanism that results in the introduction of nucleic acid into a cell, including any combination thereof. In some embodiments, transformation of a cell comprises nuclear transformation. In some embodiments, transformation of a cell comprises plasmid transformation and conjugation.

In some embodiments, when more than one nucleic acid sequence is introduced, the nucleotide sequences are assembled as part of a single nucleic acid construct, or as separate nucleic acid constructs, and are located on the same or different nucleic acid constructs. In some embodiments, nucleotide sequences are introduced into the cell of interest in a single transformation event, or in separate transformation events.

### Type I CRISPR-Cas System

In some embodiments, the Type I CRISPR-Cas system disclosed herein is a Type I-A system, Type I-B system, Type I-C system, Type I-D system, Type I-E system, or Type I-F system. In some embodiments, the Type I CRISPR-Cas system is a Type I-A system. In some embodiments, the Type I CRISPR-Cas system is a Type I-B system. In some embodiments, the Type I CRISPR-Cas system is a Type I-C system. In some embodiments, the Type I CRISPR-Cas system is a Type I-D system. In some embodiments, the Type I CRISPR-Cas system is a Type I-E system. In some embodiments, the Type I CRISPR-Cas system is a Type I-F system. In some embodiments, the Type I CRISPR-Cas system is endogenous to the target bacterium. In some embodiments, the second Type I CRISPR-Cas system is endogenous to the target bacterium. In some embodiments, the endogenous Type I CRISPR-Cas system comprises Cascade polypeptides. In some embodiments, Type I Cascade polypeptides process CRISPR arrays to produce a processed RNA that is then used to bind the complex to a target sequence that is complementary to the spacer in the processed RNA. In some embodiments, the Type I Cascade complex is a Type I-A Cascade polypeptides, a Type I-B Cascade polypeptides, a Type I-C Cascade polypeptides, a Type I-D Cascade polypeptides, a Type I-E Cascade polypeptides, a Type I-F Cascade polypeptides, or a Type I-U Cascade polypeptides.

In some embodiments, the Type I Cascade complex comprises: (a) a nucleotide sequence encoding a Cas7 (Csa2) polypeptide, a nucleotide sequence encoding a Cas8a1 (Csx13) polypeptide or a Cas8a2 (Csx9) polypeptide, a nucleotide sequence encoding a Cas5 polypeptide, a nucleotide sequence encoding a Csa5 polypeptide, a nucleotide sequence encoding a Cas6a polypeptide, a nucleotide sequence encoding a Cas3′ polypeptide, and a nucleotide sequence encoding a Cas3″ polypeptide having no nuclease activity (Type I-A); (b) a nucleotide sequence encoding a Cas6b polypeptide, a nucleotide sequence encoding a Cas8b (Csh1) polypeptide, a nucleotide sequence encoding a Cas7 (Csh2) polypeptide, and a nucleotide sequence encoding a Cas5 polypeptide (Type I-B); (c) a nucleotide sequence encoding a Cas5d polypeptide, a nucleotide sequence encoding a Cas8c (Csd1) polypeptide, and a nucleotide sequence encoding a Cas7 (Csd2) polypeptide (Type I-C); (d) a nucleotide sequence encoding a Cas10d (Csc3) polypeptide, a nucleotide sequence encoding a Csc2 polypeptide, a nucleotide sequence encoding a Csc1 polypeptide, and a nucleotide sequence encoding a Cas6d polypeptide (Type I-D); (e) a nucleotide sequence encoding a Cse1 (CasA) polypeptide, a nucleotide sequence encoding a Cse2 (CasB) polypeptide, a nucleotide sequence encoding a Cas7 (CasC) polypeptide, a nucleotide sequence encoding a Cas5 (CasD) polypeptide, and a nucleotide sequence encoding a Cas6e (CasE) polypeptide (Type I-E); and/or (f) a nucleotide sequence encoding a Cys1 polypeptide, a nucleotide sequence encoding a Cys2 polypeptide, a nucleotide sequence encoding a Cas7 (Cys3) polypeptide, and a nucleotide sequence encoding a Cas6f polypeptide (Type I-F).

In some embodiments, the Type I CRISPR-Cas system is exogenous to the target bacterium.

In some embodiments, the Type I CRISPR-Cas system comprises one or more of the components of Table 4. In some cases, the Type I CRISPR-Cas system comprises a Cas protein encoded by a polynucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of the sequences set forth in SEQ ID NOs: 1-4, 9-14, or 21-25.

In some cases, the Type I CRISPR-Cas system comprises a Cas protein comprising an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or is 100% identical to any one of the sequences set forth in SEQ ID NOs: 5-8, 15-20, or 26-30.

In some embodiments, the CRISPR-Cas system is a Type I-C CRISPR-Cas system from Pseudomonas aeruginosa (PAIC). In some cases, the PAIC system comprises a Cas3, Cas5c, Cas8c, or Cas7C, or any combination thereof. In some cases, the PAIC system comprises a Cas3 having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 5. In some cases, the PAIC system comprises a Cas3 having SEQ ID NO: 5. For example, the PAIC system comprises a Cas3 having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. In some cases, the PAIC system comprises a Cas3 having SEQ ID NO: 1. In some cases, the PAIC system comprises a Cas5c having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 6. In some cases, the PAIC system comprises a Cas5c having SEQ ID NO: 6. For example, the PAIC system comprises a Cas5c having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2. In some cases, the PAIC system comprises a Cas5c having SEQ ID NO: 2. In some cases, the PAIC system comprises a Cas8c having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 7. In some cases, the PAIC system comprises a Cas8c having SEQ ID NO: 7. For example, the PAIC system comprises a Cas8c having a sequence at least 80% 81% 82%, 83%, 84%, 85%, 86%, 87%, 88%, 89%, 90%, 91%, 92%, 93%, 94%, 95%, 96%, 97%, 98%, or 99% identical to SEQ ID NO: 3. In some cases, the PAIC system comprises a Cas8c having SEQ ID NO: 3. In some cases, the PAIC system comprises a Cas7c having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 8. In some cases, the PAIC system comprises a Cas7c having SEQ ID NO: 8. For example, the PAIC system comprises a Cas7c having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 4. In some cases, the PA1C system comprises a Cas7c having SEQ ID NO: 4.

In some embodiments, the CRISPR-Cas system is a Type I-E CRISPR-Cas system from Escherichia coli (ECIE). In some cases, the ECIE system comprises a Cas3, Cse1/CasA, Cse2/CasB, Cas7/Cse4/CasC, Cas5/CasD, or Cas6/Cse3/CasE, or any combination thereof. In some cases, the ECIE system comprises a Cas3 having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 15. In some cases, the ECIE system comprises a Cas3 having SEQ ID NO: 15. For example, the ECIE system comprises a Cas3 having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 9. In some cases, the ECIE system comprises a Cas3 having SEQ ID NO: 9. In some cases, the ECIE system comprises a Cse1/CasA having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 16. In some cases, the ECIE system comprises a Cse1/CasA having SEQ ID NO: 16. For example, the ECIE system comprises a Cse1/CasA having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 10. In some cases, the ECIE system comprises a Cse1/CasA having SEQ ID NO: 10. In some cases, the ECIE system comprises a Cse2/CasB having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 17. In some cases, the ECIE system comprises a Cse2/CasB having SEQ ID NO: 17. For example, the ECIE system comprises a Cse2/CasB having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 11. In some cases, the ECIE system comprises a Cse2/CasB having SEQ ID NO: 11. In some cases, the ECIE system comprises a Cas7/Cse4/CasC having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 18. In some cases, the ECIE system comprises a Cas7/Cse4/CasC having SEQ ID NO: 18. For example, the ECIE system comprises a Cas7/Cse4/CasC having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 12. In some cases, the ECIE system comprises a Cas7/Cse4/CasC having SEQ ID NO: 12. In some cases, the ECIE system comprises a Cas5/CasD having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 19. In some cases, the ECIE system comprises a Cas5/CasD having SEQ ID NO: 19. For example, the ECIE system comprises a Cas5/CasD having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 13. In some cases, the ECIE system comprises a Cas5/CasD having SEQ ID NO: 13. In some cases, the ECIE system comprises a Cas6/Cse3/CasE having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 20. In some cases, the ECIE system comprises a Cas6/Cse3/CasE having SEQ ID NO: 20. For example, the ECIE system comprises a Cas6/Cse3/CasE having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 14. In some cases, the ECIE system comprises a Cas6/Cse3/CasE having SEQ ID NO: 14.

In some embodiments, the CRISPR-Cas system is a Type I-F CRISPR-Cas system from Escherichia coli (ECIF). In some cases, the ECIF system comprises a Cas3, Cas8f/Csy1, Cas5/Csy2, Cas7/Csy3, or Cas6f, or any combination thereof. In some cases, the ECIF system comprises a Cas3 having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 26. In some cases, the ECIF system comprises a Cas3 having SEQ ID NO: 26. For example, the ECIF system comprises a Cas3 having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 21. In some cases, the ECIF system comprises a Cas3 having SEQ ID NO: 21. In some cases, the ECIF system comprises a Cas8f/Csy1 having a sequence at least 80%, 81%, 82%, 83%, 84%, 85%, 86%, 87%, 88%, 89%, 90%, 91%, 92%, 93% 94%, 95%, 96%, 97%, 98%, or 99% identical to SEQ ID NO: 27. In some cases, the ECIF system comprises a Cas8f/Csy1 having SEQ ID NO: 27. For example, the ECIF system comprises a Cas8f/Csy1 having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 22. In some cases, the ECIF system comprises a Cas8f/Csy1 having SEQ ID NO: 22. In some cases, the ECIF system comprises a Cas5/Csy2 having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 28. In some cases, the ECIF system comprises a Cas5/Csy2 having SEQ ID NO: 28. For example, the ECIF system comprises a Cas5/Csy2 having a sequence at least 80%, 81%, 82%, 83%, 84%, 85%, 86%, 87%, 88%, 89%0, 90%, 91%, 92%, 93%, 94%, 95%, 96%, 97%, 98%, or 99% identical to SEQ ID NO: 23. In some cases, the ECIF system comprises a Cas5/Csy2 having SEQ ID NO: 23. In some cases, the ECIF system comprises a Cas7/Csy3 having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 29. In some cases, the ECIF system comprises a Cas7/Csy3 having SEQ ID NO: 29. For example, the ECIF system comprises a Cas7/Csy3 having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 24. In some cases, the ECIF system comprises a Cas7/Csy3 having SEQ ID NO: 24. In some cases, the ECIF system comprises a Cas6f having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 30. In some cases, the ECIF system comprises a Cas6f having SEQ ID NO: 30. For example, the ECIF system comprises a Cas6f having a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 25. In some cases, the ECIF system comprises a Cas6f having SEQ ID NO: 25.

TABLE 4
Example Type I CRISPR-Cas system components

| | SEQ | |
|---|---|---|
| | ID | |
| | NO | SEQUENCE (5′-3′) |
| Cas3 | 1 | ATGGACGCGGAGGCTAGCGATACTCACTTTTTTGCTCACTCCACCTTAAAGGCA |
| | | GATCGCAGCGATTGGCAGCCTCTGGTCGAGCATCTACAGGCTGTTGCCCGTTTG |
| | | GCAGGAGAGAAGGCTGCCTTCTTCGGCGGCGGTGAATTAGCTGCTCTTGCTGGT |
| | | CTGTTGCATGACTTGGGTAAATACACTGACGAGTTTCAGCGGCGTATTGCGGGT |
| | | GATGCCATCCGTGTCGATCACTCTACTCGCGGGGCCATACTGGCGGTAGAACGC |
| | | TATGGCGCGCTAGGTCAATTGCTAGCCTACGGCATCGCTGGCCACCATGCCGGG |
| | | TTGGCCAATGGCCGCGAGGCTGGTGAGCGAACTGCCTTGGTCGACCGCCTGAA |
| | | AGGGGTTGGGCTGCCACGGTTATTGGAGGGGTGGTGCGTGGAAATCGTGCTAC |
| | | CCGAGCGCCTTCAACCACCGCCACTAAAAGCGCGCCTGGAAAGAGGTTTCTTTC |
| | | AGTTGGCCTTTCTTGGCCGGATGCTCTTTTCCTGCTTGGTTGATGCGGATTATCT |
| | | AGATACCGAAGCCTTCTACCACCGCGTCGAAGGACGGCGCTCCCTTCGCGAGC |
| | | AAGCGCGGCCGACCTTGGCCGAGTTACGCGCAGCCCTTGATCGGCATCTGACTG |
| | | AGTTCAAGGGAGATACGCCGGTCAACCGCGTTCGCGGGGAGATATTGGCCGGC |
| | | GTGCGCGGCAAGGCGAGCGAACTTCCCGGGCTGTTTTCTCTCACAGTGCCCACA |
| | | GGAGGCGGCAAGACCCTGGCCTCTCTGGCTTTCGCCCTGGATCACGCTCTAGCT |
| | | CATGGGCTGCGCCGGGTGATCTACGTGATTCCCTTCACTAGCATCGTCGAGCAG |
| | | AACGCTGCGGTATTCCGTCGTGCACTCGGGGCCTTAGGCGAAGAGGCGGTGCT |
| | | GGAGCATCACAGCGCCTTCGTTGATGACCGCCGGCAGAGCCTGGAGGCCAAGA |
| | | AGAAACTGAACCTAGCGATGGAGAACTGGGACGCGCCTATCGTGGTGACCACT |
| | | GCAGTGCAGTTCTTCGAAAGCCTGTTTGCCGACCGTCCAGCCCAGTGCCGCAAG |
| | | CTACACAACATCGCCGGCAGCGTGGTGATTCTTGACGAGGCACAGACCCTACC |
| | | GCTCAAGCTGTTGCGGCCCTGCGTTGCCGCCCTTGATGAACTGGCGCTCAACTA |
| | | CCGTTGTAGCCCAGTTCTCTGTACTGCCACGCAGCCAGCGCTTCAATCGCCGGA |
| | | TTTCATCGGTGGGCTGCAGGACGTACGTGAGCTGGCGCCCGAGCCGCAGCGGC |
| | | TGTTCCGGGAGTTGGTGCGGGTACGAATACGGACATTGGGCCCGCTCGAAGAT |
| | | GCGGCCTTGACTGAGCAGATCGCCAGGCGTGAACAAGTGCTGTGCATCGTCAA |
| | | CAATCGACGCCAGGCCCGTGCGCTCTATGAGTCGCTTGCCGAGTTGCCCGGTGC |
| | | CCGCCATCTCACCACCCTGATGTGCGCCAAGCACCGTAGCAGCGTGCTGGCCGA |
| | | GGTGCGCCAGATGCTCAAAAAGGGGGAGCCCTGTCGCCTGGTGGCCACCTCGC |
| | | TGATCGAGGCCGGTGTGGATGTGGATTTTCCCGTGGTACTGCGTGCCGAGGCTG |
| | | GATTGGATTCCATCGCCCAGGCCGCGGGACGCTGCAATCGCGAAGGCAAGCGG |
| | | CCGCTGGCCGAAAGCGAGGTGCTGGTGTTCGCCGCGGCCAATTCTGACTGGGC |
| | | GCCACCCGAGGAACTCAAGCAGTTCGCCCAGGCCGCCCGCGAAGTGATGCGCC |
| | | TGCACCCGGATGATTGCCTGTCCATGGCGGCCATCGAGCGGTATTTTCGCATAC |
| | | TGTACTGGCAGAAGGGCGCGGAGGAGTTGGATGCGGGTAACCTGCTCGGCCTG |
| | | ATTGAGAGAGGCCGGCTCGATGGCCTGCCCTACGAGACTTTGGCCACCAAGTTC |
| | | CGCATGATCGACAGCCTTCAACTGCCGGTGATCATCCCATTTGATGACGAGGCC |
| | | AGAGCAGCCCTGCGCGAGCTGGAGTTCGCCGACGGCTGCGCCGCCATCGCCCG |
| | | TCGCCTGCAGCCATATCTGGTGCAGATGCCACGCAAGGGTTATCAGGCATTGCG |
| | | GGAAGCCGGTGCGATCCAGGCGGCGGCAGGTACGCGTTATGGTGAGCAGTTTA |
| | | TGGCGTTGGTCAACCCTGATCTGTATCACCACCAATTCGGGTTGCACTGGGATA |
| | | ATCCGGCCTTTGTCAGCAGCGAGCGGCTATGTTGGTAG |
| Cas5c | 2 | ATGGCCTACGGAATTCGCTTAATGGTCTGGGGCGAGCGTGCCTGCTTCACCCGC |
| | | CCGGAAATGAAGGTGGAACGCGTCTCTTACGATGCGATCACGCCGTCCGCCGC |
| | | GCGCGGCATTCTCGAGGCTATCCACTGGAAGCCGGCGATTCGCTGGGTGGTGG |
| | | ATCGCATTCAAGTGCTTAAGCCGATCCGCTTCGAATCCATCCGGCGCAACGAGG |
| | | TCGGCGGCAAGCTGTCCGCTGTCAGCGTCGGTAAGGCAATGAAGGCCGGGCGT |
| | | ACTAATGGTCTGGTGAATCTGGTCGAGGAGGATCGCCAGCAGCGCGCGACTAC |
| | | TCTGCTGCGCGATGTCTCCTATGTCATCGAGGCGCATTTCGAGATGACTGACAG |
| | | GGCTGGCGCCGACGATACGGTGGGCAAGCATCTGGATATCTTCAACCGTCGCG |
| | | CACGGAAGGGGCAGTGCTTCCATACACCCTGCCTAGGCGTGCGCGAGTTTCCGG |
| | | CCAGTTTTCGGTTGCTGGAAGAGGGCAGTGCCGAGCCTGAAGTCGATGCCTTTC |
| | | TGCGCGGCGAGCGTGATCTGGGCTGGATGCTGCATGACATTGACTTCGCCGATG |
| | | GCATGACCCCGCACTTCTTCCGTGCCCTGATGCGCGATGGGCTGATCGAGGTGC |
| | | CGGCCTTCAGGGCGGCAGAGGACAAGGCATGA |
| Cas8c | 3 | ATGATCCTTTCGGCCCTCAATGACTATTATCAGCGACTGCTGGAGCGGGGTGAA |
| | | GCGAATATCTCACCCTTCGGCTACAGCCAAGAAAAGATCAGTTACGCCCTGCTG |
| | | CTGTCCGCACAAGGAGAGTTGCTGGACGTGCAGGACATTCGCTTGCTCTCTGGC |
| | | AAGAAGCCTCAACCCAGGCTTATGAGTGTGCCGCAGCCGGAGAAGCGCACCTC |
| | | GGGCATCAAGTCCAACGTACTGTGGGACAAGACCAGCTATGTGCTGGGTGTTA |
| | | GTGCCAAGGGCGGAGAGCGTACTCAGCAGGAGCACGAGTCCTTCAAGACGCTG |
| | | CACCGGCAGATCTTGGTTGGGGAAGGCGACCCCGGTCTGCAGGCCTTGCTCCAG |
| | | TTCCTCGACTGTTGGCAGCCGGAGCAGTTCAAGCCCCCGCTGTTCAGCGAAGCA |
| | | ATGCTCGACAGCAACTTAGTGTTCCGCCTAGACGGCCAACAACGCTATCTGCAC |
| | | GAGACTCCGGCGGCCCTGGCGTTGCGTACCCGGCTGTTGGCCGACGGCGACAG |
| | | CCGCGAGGGGCTGTGCCTAGTCTGCGGCCAACGTCAGCCGTTGGCGCGCCTGCA |
| | | TCCAGCGGTCAAGGGCGTCAATGGTGCCCAGAGTTCGGGGGCTTCCATCGTCTC |
| | | CTTCAACCTCGACGCTTTTTCCTCCTACGGCAAGAGCCAGGGGGAAAATGCTCC |
| | | GGTCTCCGAACAGGCCGCCTTTGCCTACACCACGGTGCTCAACCATTTGTTGCG |
| | | TCGCGACGAGCACAACCGCCAGCGCCTGCAGATTGGCGACGCGAGTGTGGTGT |
| | | TCTGGGCGCAGGCGGATACTCCTGCTCAGGTGGCCGCCGCCGAGTCGACCTTCT |
| | | GGAACCTGCTGGAGCCACCCGCAGATGATGGTCAGGAAGCGGAAAAGCTGCGC |
| | | GGCGTGCTGGATGCTGTGGCCACGGGGCGGCCCTTGCATGAGCTCGACTCGCTA |
| | | ATGGAGGAAGGTACCCGCATTTTTGTGTTAGGGCTGGCGCCCAATACCTCGCGA |
| | | CTGTCCATTCGGTTCTGGGCAGTCGATAGCCTTGCGGTATTCACCCAGCATCTG |
| | | GCCGAGCATTTCCGGGATATGCACCTTGAGCCTCTGCCCTGGAAGACGGAGCCG |
| | | GCCATCTGGCGCTTGCTCTATGCTACCGCGCCCAGTCGTGACGGCAGAGCCAAG |
| | | ACCGAAGACGTACTCCCACAACTGGCCGGTGAAATGACCCGCGCCATCCTGAC |
| | | CGGCAGCCGCTATCCGCGCAGTTTGCTAGCCAACCTGATCATGCGCATGCGTGC |
| | | CGACGGCGACGTCTCTGGCATACGCGTCGCGCTGTGCAAGGCCGTGCTCGCTCG |
| | | CGAGGCACGCCTGAGCGGCAAAATTCACCAAGAGGAGCTACCTATGAGTCTCG |
| | | ACAAGGACGCCAGCAACCCCGGCTATCGCTTGGGGAGGCTGTTCGCCGTGTTG |
| | | GAAGGCGCCCAGCGCGCAGCCCTGGGCGACAGGGTCAATGCCACTATCCGTGA |
| | | CCGCTACTACGGTGCCGCGTCCAGCACGCCAGCCACGGTTTTCCCGATACTGCT |
| | | GCGCAACACACAAAACCACTTGGCCAAGCTGCGCAAGGAGAAGCCCGGACTAG |
| | | CAGTGAACCTAGAGCGCGATATAGGCGAAATCATTGACGGTATGCAGAGCCAA |
| | | TTCCCGCGTTGCCTGCGCCTGGAGGACCAGGGACGCTTTGCTATTGGTTACTAC |
| | | CAACAGGCCCAGGCCCGTTTCAACCGTGGCCCCGATTCCGTCGAGTAA |
| Cas7c | 4 | ATGACCGCCATCTCCAACCGCTACGAGTTCGTTTACCTCTTTGATGTCAGCAAT |
| | | GGCAATCCCAATGGCGACCCGGATGCTGGCAACATGCCGCGTCTCGATCCGGA |
| | | AACCAACCAGGGGTTGGTCACTGACGTTTGCCTCAAGCGCAAGATCCGCAACT |
| | | ACGTCAGCCTGGAGCAGGAAAGTGCCCCCGGCTATGCCATCTATATGCAGGAA |
| | | AAATCCGTGCTGAATAACCAGCACAAACAGGCCTACGAGGCGCTCGGTATCGA |
| | | GTCAGAGGCAAAGAAACTGCCCAAGGACGAAGCCAAGGCGCGCGAACTGACC |
| | | TCTTGGATGTGCAAGAACTTCTTCGATGTGCGTGCTTTCGGGGCGGTGATGACC |
| | | ACCGAGATTAATGCCGGCCAGGTGCGTGGACCGATCCAACTGGCATTCGCCAC |
| | | GTCTATCGACCCGGTATTGCCTATGGAGGTATCCATCACCCGCATGGCGGTGAC |
| | | TAACGAAAAGGATTTGGAGAAGGAACGCACCATGGGACGCAAGCACATCGTGC |
| | | CTTACGGCTTGTACCGCGCCCATGGTTTCATCTCTGCCAAGTTGGCCGAGCGAA |
| | | CCGGCTTTTCCGACGACGACTTGGAACTGCTATGGCGCGCTTTGGCCAATATGT |
| | | TCGAACACGACCGCTCGGCGGCACGTGGCGAGATGGCAGCGCGCAAGTTGATC |
| | | GTCTTCAAGCATGAGCATGCCATGGGCAATGCACCCGCCCATGTGCTGTTCGGC |
| | | AGCGTTAAGGTCGAGCGAGTCGAGGGGGACGCAGTTACACCAGCACGCGGTTT |
| | | CCAGGATTACCGTGTCAGCATCGATGCGGAAGCTCTGCCTCAGGGCGTGAGCGT |
| | | GCGCGAGTACCTCTAG |
| Cas3 | 5 | MDAEASDTHFFAHSTLKADRSDWQPLVEHLQAVARLAGEKAAFFGGGELAALAG |
| | | LLHDLGKYTDEFQRRIAGDAIRVDHSTRGAILAVERYGALGQLLAYGIAGHHAGLA |
| | | NGREAGERTALVDRLKGVGLPRLLEGWCVEIVLPERLQPPPLKARLERGFFQLAFL |
| | | GRMLFSCLVDADYLDTEAFYHRVEGRRSLREQARPTLAELRAALDRHLTEFKGDT |
| | | PVNRVRGEILAGVRGKASELPGLFSLTVPTGGGKTLASLAFALDHALAHGLRRVIY |
| | | VIPFTSIVEQNAAVFRRALGALGEEAVLEHHSAFVDDRRQSLEAKKKLNLAMENW |
| | | DAPIVVTTAVQFFESLFADRPAQCRKLHNIAGSVVILDEAQTLPLKLLRPCVAALDE |
| | | LALNYRCSPVLCTATQPALQSPDFIGGLQDVRELAPEPQRLFRELVRVRIRTLGPLE |
| | | DAALTEQIARREQVLCIVNNRRQARALYESLAELPGARHLTTLMCAKHRSSVLAEV |
| | | RQMLKKGEPCRLVATSLIEAGVDVDFPVVLRAEAGLDSIAQAAGRCNREGKRPLA |
| | | ESEVLVFAAANSDWAPPEELKQFAQAAREVMRLHPDDCLSMAAIERYFRILYWQK |
| | | GAEELDAGNLLGLIERGRLDGLPYETLATKFRMIDSLQLPVIIPFDDEARAALRELEF |
| | | ADGCAAIARRLQPYLVOMPRKGYQALREAGAIQAAAGTRYGEQFMALVNPDLYH |
| | | HQFGLHWDNPAFVSSERLCW* |
| Cas5c | 6 | MAYGIRLMVWGERACFTRPEMKVERVSYDAITPSAARGILEAIHWKPAIRWVVDRI |
| | | QVLKPIRFESIRRNEVGGKLSAVSVGKAMKAGRTNGLVNLVEEDRQQRATTLLRD |
| | | VSYVIEAHFEMTDRAGADDTVGKHLDIFNRRARKGQCFHTPCLGVREFPASFRLLE |
| | | EGSAEPEVDAFLRGERDLGWMLHDIDFADGMTPHFFRALMRDGLIEVPAFRAAED |
| | | KA* |
| Cas8c | 7 | MILSALNDYYQRLLERGEANISPFGYSQEKISYALLLSAQGELLDVQDIRLLSGKKP |
| | | QPRLMSVPQPEKRTSGIKSNVLWDKTSYVLGVSAKGGERTQQEHESFKTLHRQILV |
| | | GEGDPGLQALLQFLDCWQPEQFKPPLFSEAMLDSNLVFRLDGQQRYLHETPAALA |
| | | LRTRLLADGDSREGLCLVCGQRQPLARLHPAVKGVNGAQSSGASIVSFNLDAFSSY |
| | | GKSQGENAPVSEQAAFAYTTVLNHLLRRDEHNRQRLQIGDASVVFWAQADTPAQ |
| | | VAAAESTFWNLLEPPADDGQEAEKLRGVLDAVATGRPLHELDSLMEEGTRIFVLG |
| | | LAPNTSRLSIRFWAVDSLAVFTQHLAEHFRDMHLEPLPWKTEPAIWRLLYATAPSR |
| | | DGRAKTEDVLPQLAGEMTRAILTGSRYPRSLLANLIMRMRADGDVSGIRVALCKA |
| | | VLAREARLSGKIHQEELPMSLDKDASNPGYRLGRLFAVLEGAQRAALGDRVNATI |
| | | RDRYYGAASSTPATVFPILLRNTQNHLAKLRKEKPGLAVNLERDIGEIIDGMQSQFP |
| | | RCLRLEDQGRFAIGYYQQAQARFNRGPDSVE* |
| Cas7c | 8 | MTAISNRYEFVYLFDVSNGNPNGDPDAGNMPRLDPETNQGLVTDVCLKRKIRNYV |
| | | SLEQESAPGYAIYMQEKSVLNNQHKQAYEALGIESEAKKLPKDEAKARELTSWMC |
| | | KNFFDVRAFGAVMTTEINAGQVRGPIQLAFATSIDPVLPMEVSITRMAVTNEKDLE |
| | | KERTMGRKHIVPYGLYRAHGFISAKLAERTGFSDDDLELLWRALANMFEHDRSAA |
| | | RGEMAARKLIVFKHEHAMGNAPAHVLFGSVKVERVEGDAVTPARGFQDYRVSID |
| | | AEALPQGVSVREYL* |
| Cas3 | 9 | ATGGAACCTTTTAAATATATATGCCATTACTGGGGAAAATCCTCAAAAAGCTTG |
| | | ACGAAAGGAAATGATATTCATCTGTTAATTTATCATTGCCTTGATGTTGCTGCTG |
| | | TTGCAGATTGCTGGTGGGATCAATCAGTCGTACTGCAAAATACTTTTTGCCGAA |
| | | ATGAAATGCTATCAAAACAGAGGGTGAAGGCCTGGCTGTTATTTTTCATTGCTC |
| | | TTCATGATATTGGAAAGTTTGATATACGATTCCAATATAAATCAGCAGAAAGTT |
| | | GGCTGAAATTAAATCCTGCAACGCCATCACTTAATGGTCCATCAACACAAATGT |
| | | GCCGTAAATTTAATCATGGTGCAGCCGGTCTGTATTGGTTTAACCAGGATTCAC |
| | | TTTCAGAGCAATCTCTCGGGGATTTTTTCAGTTTTTTTGATGCCGCTCCTCATCC |
| | | TTATGAGTCCTGGTTTCCATGGGTAGAGGCCGTTACAGGACATCATGGTTTTAT |
| | | ATTACATTCCCAGGATCAAGATAAGTCGCGTTGGGAAATGCCAGCTTCTCTGGC |
| | | ATCTTATGCTGCGCAAGATAAACAGGCTCGTGAGGAGTGGATATCTGTACTGGA |
| | | AGCATTATTTTTAACGCCAGCGGGGTTATCTATAAACGATATACCACCTGATTG |
| | | TTCATCACTGTTAGCAGGTTTTTGCTCGCTTGCTGACTGGTTAGGCTCCTGGACT |
| | | ACAACGAATACCTTTCTGTTTAATGAGGATGCGCCTTCCGACATAAATGCTCTG |
| | | AGAACGTATTTCCAGGACCGACAGCAGGATGCGAGCCGGGTATTGGAGTTGAG |
| | | TGGACTTGTATCAAATAAGCGATGTTATGAAGGTGTTCATGCACTACTGGACAA |
| | | TGGCTATCAACCCAGACAATTACAGGTGTTAGTTGATGCTCTTCCAGTAGCTCC |
| | | CGGGCTGACGGTAATAGAGGCACCTACAGGCTCCGGTAAAACGGAAACAGCGC |
| | | TGGCCTATGCTTGGAAACTTATTGATCAACAAATTGCGGATAGTGTTATTTTTGC |
| | | CCTCCCAACACAAGCTACCGCGAATGCTATGCTTACGAGAATGGAAGCGAGCG |
| | | CGAGCCACTTATTTTCATCCCCAAATCTTATTCTTGCTCATGGCAATTCACGGTT |
| | | TAACCACCTCTTTCAATCAATAAAATCACGCGCGATTACTGAACAGGGGCAAG |
| | | AAGAAGCGTGGGTTCAGTGTTGTCAGTGGTTGTCACAAAGCAATAAGAAAGTG |
| | | TTTCTTGGGCAAATCGGCGTTTGCACGATTGATCAGGTGTTGATATCGGTATTG |
| | | CCAGTTAAACACCGCTTTATCCGTGGTTTGGGAATTGGTCGAAGTGTTTTAATT |
| | | GTTGATGAAGTTCATGCTTACGACACCTATATGAACGGCTTGCTGGAGGCAGTG |
| | | CTCAAGGCTCAGGCTGATGTGGGAGGGAGTGTTATTCTTCTTTCCGCAACCCTA |
| | | CCAATGAAACAAAAACAGAAACTTCTGGATACTTATGGTCTGCATACAGATCC |
| | | AGTGGAAAATAACTCCGCATATCCACTCATTAACTGGCGAGGTGTGAATGGTGC |
| | | GCAACGTTTTGATCTGCTAGCTCATCCAGAACAACTCCCGCCCCGCTTTTCGATT |
| | | CAGCCAGAACCTATTTGTTTAGCTGACATGTTACCTGACCTTACGATGTTAGAG |
| | | CGAATGATCGCAGCGGCAAACGCGGGTGCACAGGTCTGTCTTATTTGCAATTTG |
| | | GTTGACGTTGCACAAGTATGCTACCAACGGCTAAAGGAGCTAAATAACACGCA |
| | | AGTAGATATAGATTTGTTTCATGCGCGCTTTACGCTGAACGATCGTCGTGAAAA |
| | | AGAGAATCGAGTTATTAGCAATTTCGGCAAAAATGGGAAGCGAAATGTTGGAC |
| | | GGATACTTGTCGCAACCCAGGTCGTGGAACAATCACTCGACGTTGATTTTGATT |
| | | GGTTAATTACTCAGCATTGTCCTGCAGATTTGCTTTTCCAACGATTGGGCCGTTT |
| | | ACATCGCCATCATCGCAAATATCGTCCCGCTGGTTTTGAGATTCCTGTTGCCACC |
| | | ATTTTGCTGCCTGATGGCGAGGGTTACGGACGACATGAGCATATTTATAGCAAC |
| | | GTTAGAGTCATGTGGCGGACGCAGCAACATATTGAGGAGCTTAATGGAGCATC |
| | | CTTATTTTTCCCTGATGCTTACCGGCAATGGCTGGATAGCATTTACGATGATGCG |
| | | GAAATGGATGAGCCAGAATGGGTCGGCAATGGCATGGATAAATTTGAAAGCGC |
| | | CGAGTGTGAAAAAAGGTTCAAGGCTCGCAAGGTCCTGCAGTGGGCTGAAGAAT |
| | | ATAGCTTGCAGGATAACGATGAAACCATTCTTGCGGTAACGAGGGATGGGGAA |
| | | ATGAGCCTGCCATTATTGCCTTATGTACAAACGTCTTCAGGTAAACAACTGCTC |
| | | GATGGCCAGGTCTACGAGGACCTAAGTCATGAACAGCAGTATGAGGCGCTTGC |
| | | ACTTAATCGCGTCAATGTACCCTTCACCTGGAAACGTAGTTTTTCTGAAGTAGT |
| | | AGATGAAGATGGGTTACTTTGGCTGGAAGGGAAACAGAATCTGGATGGATGGG |
| | | TCTGGCAGGGTAACAGTATTGTTATTACCTATACAGGGGATGAAGGGATGACC |
| | | AGAGTCATCCCTGCAAATCCCAAATAA |
| Cse1/ | 10 | ATGAATTTGCTTATTGATAACTGGATCCCTGTACGCCCGCGAAACGGGGGGAAA |
| CasA | | GTCCAAATCATAAATCTGCAATCGCTATACTGCAGTAGAGATCAGTGGCGATTA |
| | | AGTTTGCCCCGTGACGATATGGAACTGGCCGCTTTAGCACTGCTGGTTTGCATT |
| | | GGGCAAATTATCGCCCCGGCAAAAGATGACGTTGAATTTCGACATCGCATAAT |
| | | GAATCCGCTCACTGAAGATGAGTTTCAACAACTCATCGCGCCGTGGATAGATAT |
| | | GTTCTACCTTAATCACGCAGAACATCCCTTTTATGCAGACCAAAGGTGTCAAAGC |
| | | AAATGATGTGACTCCAATGGAAAAACTGTTGGCTGGGGTAAGCGGCGCGACGA |
| | | ATTGTGCATTTGTCAATCAACCGGGGCAGGGTGAAGCATTATGTGGTGGATGCA |
| | | CTGCGATTGCGTTATTCAACCAGGCGAATCAGGCACCAGGTTTTGGTGGTGGTT |
| | | TTAAAAGCGGTTTACGTGGAGGAACACCTGTAACAACGTTCGTACGTGGGATC |
| | | GATCTTCGTTCAACGGTGTTACTCAATGTCCTCACATTACCTCGTCTTCAAAAAC |
| | | AATTTCCTAATGAATCACATACGGAAAACCAACCTACCTGGATTAAACCTATCA |
| | | AGTCCAATGAGTCTATACCTGCTTCGTCAATTGGGTTTGTCCGTGGTCTATTCTG |
| | | GCAACCAGCGCATATTGAATTATGCGATCCCATTGGGATTGGTAAATGTTCTTG |
| | | CTGTGGACAGGAAAGCAATTTGCGTTATACCGGTTTTCTTAAGGAAAAATTTAC |
| | | CTTTACAGTTAATGGGCTATGGCCCCATCCGCATTCCCCTTGTCTGGTAACAGTC |
| | | AAGAAAGGGGAGGTTGAGGAAAAATTTCTTGCTTTCACCACCTCCGCACCATCA |
| | | TGGACACAAATCAGCCGAGTTGTGGTAGATAAGATTATTCAAAATGAAAATGG |
| | | AAATCGCGTGGCGGCGGTTGTGAATCAATTCAGAAATATTGCGCCGCAAAGTC |
| | | CTCTTGAATTGATTATGGGGGGATATCGTAATAATCAAGCATCTATTCTTGAAC |
| | | GGCGTCATGATGTGTTGATGTTTAATCAGGGGTGGCAACAATACGGCAATGTGA |
| | | TAAACGAAATAGTGACTGTTGGTTTGGGATATAAAACAGCCTTACGCAAGGCG |
| | | TTATATACCTTTGCAGAAGGGTTTAAAAATAAAGACTTCAAAGGGGCCGGAGT |
| | | CTCTGTTCATGAGACTGCAGAAAGGCATTTCTATCGACAGAGTGAATTATTAAT |
| | | TCCCGATGTACTGGCGAATGTTAATTTTTCCCAGGCTGATGAGGTAATAGCTGA |
| | | TTTACGAGACAAACTTCATCAATTGTGTGAAATGCTATTTAATCAATCTGTAGC |
| | | TCCCTATGCACATCATCCTAAATTAATAAGCACATTAGCGCTTGCCCGCGCCAC |
| | | GCTATACAAACATTTACGGGAGTTAAAACCGCAAGGAGGGCCATCAAATGGCT |
| | | GA |
| Cse2/ | 11 | ATGGCTGATGAAATTGATGCAATGGCTTTATATCGAGCCTGGCAACAACTGGAT |
| CasB | | AATGGATCATGTGCGCAAATTAGACGTGTTTCAGAACCTGATGAATTACGCGAT |
| | | ATCCCTGCGTTTTATAGGCTGGTGCAACCTTTTGGTTGGGAAAACCCACGTCAC |
| | | CAGCAGGCTCTTTTGCGCATGGTGTTTTGCCTGAGCGCAGGAAAGAATGTCATC |
| | | CGACATCAGGACAAAAAATCGGAGCAAACAACAGGTATCTCGTTGGGAAGAGC |
| | | TTTAGCCAATAGTGGAAGAATTAACGAGCGCCGTATCTTTCAATTAATTCGGGC |
| | | TGACAGAACAGCCGATATGGTCCAGTTACGTCGATTACTTACTCACGCCGAACC |
| | | CGTACTTGACTGGCCATTAATGGCCAGGATGTTGACCTGGTGGGGAAAGCGCG |
| | | AACGCCAGCAACTTCTGGAAGATTTTGTATTGACCACAAACAAAAATGCGTAA |
| Cas7/ | 12 | ATGTCTAACTTTATCAATATTCATGTTCTGATCTCTCACAGCCCTTCATGTCTGA |
| Cse4/ | | ACCGCGACGATATGAACATGCAGAAAGACGCTATTTTCGGCGGCAAAAGACGA |
| CasC | | GTAAGAATTTCAAGTCAAAGCCTTAAACGTGCGATGCGTAAAAGTGGTTATTAC |
| | | GCACAAAATATTGGTGAATCCAGTCTCAGAACCATTCATCTTGCACAATTACGT |
| | | GATGTTCTTCGGCAAAAACTTGGTGAACGTTTTGACCAAAAAATCATCGATAAG |
| | | ACATTAGCGCTGCTCTCCGGTAAATCAGTTGATGAAGCCGAAAAGATTTCTGCC |
| | | GATGCGGTTACTCCCTGGGTTGTGGGAGAAATAGCCTGGTTCTGTGAGCAGGTT |
| | | GCAAAAGCAGAGGCTGATAATCTGGATGATAAAAAGCTGCTCAAAGTTCTTAA |
| | | GGAAGATATTGCCGCCATACGTGTGAATTTACAGCAGGGTGTTGATATTGCGCT |
| | | TAGTGGAAGAATGGCAACCAGCGGCATGATGACTGAGTTGGGAAAAGTTGATG |
| | | GTGCAATGTCCATTGCGCATGCGATCACTACTCATCAGGTTGATTCTGATATTG |
| | | ACTGGTTCACCGCTGTAGATGATTTACAGGAACAAGGTTCTGCACATCTGGGAA |
| | | CTCAGGAATTTTCATCGGGTGTTTTTTATCGTTATGCCAACATTAACCTCGCTCA |
| | | ACTTCAGGAAAATTTAGGTGGTGCCTCCAGGGAGCAGGCTCTGGAAATTGCAA |
| | | CCCATGTTGTTCATATGCTGGCAACAGAGGTCCCTGGAGCAAAACAGCGTACTT |
| | | ATGCCGCTTTTAACCCTGCGGATATGGTAATGGTTAATTTCTCCGATATGCCACT |
| | | TTCTATGGCAAATGCTTTTGAAAAAGCGGTTAAAGCGAAAGATGGCTTTTTGCA |
| | | ACCGTCTATACAGGCGTTTAATCAATATTGGGATCGCGTTGCCAATGGATATGG |
| | | TCTGAACGGAGCTGCTGCGCAATTCAGCTTATCTGATGTAGACCCAATTACTGC |
| | | TCAAGTTAAACAAATGCCTACTTTAGAACAGTTAAAATCCTGGGTTCGTAATAA |
| | | TGGCGAGGCGTGA |
| Cas5/ | 13 | ATGAGATCTTATTTGATCTTGCGGCTTGCTGGGCCAATGCAAGCCTGGGGGCAG |
| CasD | | CCGACCTTTGAAGGAACGCGACCTACCGGAAGATTTCCGACCCGAAGCGGGTT |
| | | ATTAGGGCTACTCGGGGCTTGTCTTGGGATCCAACGTGATGATACTTCTTCATT |
| | | ACAGGCGTTATCAGAGAGTGTGCAATTTGCAGTGCGCTGCGATGAACTCATTCT |
| | | TGACGATCGTCGTGTGTCTGTAACGGGGTTGCGTGATTACCATACAGTCCTTGG |
| | | AGCGCGAGAAGATTACCGTGGTTTGAAAAGTCATGAAACGATTCAAACATGGC |
| | | GCGAATATTTATGTGATGCCTCCTTTACCGTCGCTCTCTGGTTAACACCCCATGC |
| | | AACGATGGTTATCTCAGAACTTGAAAAAGCAGTATTAAAGCCTCGGTATACACC |
| | | TTACCTGGGGCGGAGAAGTTGCCCACTAACACACCCGCTTTTTTTGGGGACATG |
| | | TCAGGCATCGGATCCTCAGAAGGCGCTATTAAATTATGAGCCCGTTGGCGGCGA |
| | | TATATATAGTGAGGAATCAGTTACAGGGCATCATTTAAAATTTACGGCGCGCGA |
| | | CGAACCGATGATCACCTTGCCTCGACAATTTGCTTCCCGAGAATGGTATGTGAT |
| | | TAAAGGAGGTATGGATGTATCTCAGTAA |
| Cas6/ | 14 | ATGTATCTCAGTAAAGTCATCATTGCCAGGGCCTGGAGCAGGGATCTTTACCAA |
| Cse3/ | | CTTCACCAGGGATTATGGCATTTATTTCCAAACAGACCGGATGCTGCTCGTGAT |
| CasE | | TTTCTTTTTCATGTTGAGAAGCGAAACACACCAGAAGGCTGTCATGTTTTATTGC |
| | | AGTCAGCGCAAATGCCTGTTTCAACTGCCGTTGCGACAGTCATTAAAACTAAAC |
| | | AGGTTGAATTTCAACTTCAGGTTGGTGTTCCACTCTATTTTCGGCTTCGGGCAAA |
| | | TCCGATCAAAACTATTCTCGACAATCAAAAGCGCCTGGACAGTAAAGGGAATA |
| | | TTAAACGCTGTCGGGTTCCGTTAATAAAAGAAGCAGAACAAATCGCGTGGTTG |
| | | CAACGTAAATTGGGCAATGCGGCGCGCGTTGAAGATGTGCATCCCATATCGGA |
| | | ACGGCCACAGTATTTTTCTGGTGATGGTAAAAGTGGAAAGATCCAAACGGTTTG |
| | | CTTTGAAGGTGTGCTCACCATCAACGACGCGCCAGCGTTAATAGATCTTGTACA |
| | | GCAAGGTATTGGGCCAGCTAAATCGATGGGATGTGGCTTGCTATCTTTGGCTCC |
| | | ACTGTGA |
| Cas3 | 15 | MEPFKYICHYWGKSSKSLTKGNDIHLLIYHCLDVAAVADCWWDQSVVLQNTFCR |
| | | NEMLSKQRVKAWLLFFIALHDIGKFDIRFQYKSAESWLKLNPATPSLNGPSTQMCR |
| | | KFNHGAAGLYWFNQDSLSEQSLGDFFSFFDAAPHPYESWFPWVEAVTGHHGFILH |
| | | SQDQDKSRWEMPASLASYAAQDKQAREEWISVLEALFLTPAGLSINDIPPDCSSLLA |
| | | GFCSLADWLGSWTTTNTFLFNEDAPSDINALRTYFQDRQQDASRVLELSGLVSNKR |
| | | CYEGVHALLDNGYQPRQLQVLVDALPVAPGLIVIEAPTGSGKTETALAYAWKLID |
| | | QQIADSVIFALPTQATANAMLTRMEASASHLFSSPNLILAHGNSRFNHLFQSIKSRAI |
| | | TEQGQEEAWVQCCQWLSQSNKKVFLGQIGVCTIDQVLISVLPVKHRFIRGLGIGRS |
| | | VLIVDEVHAYDTYMNGLLEAVLKAQADVGGSVILLSATLPMKQKQKLLDTYGLH |
| | | TDPVENNSAYPLINWRGVNGAQRFDLLAHPEQLPPRFSIQPEPICLADMLPDLTMLE |
| | | RMIAAANAGAQVCLICNLVDVAQVCYQRLKELNNTQVDIDLFHARFTLNDRREKE |
| | | NRVISNFGKNGKRNVGRILVATQVVEQSLDVDFDWLITQHCPADLLFQRLGRLHR |
| | | HHRKYRPAGFEIPVATILLPDGEGYGRHEHIYSNVRVMWRTQQHIEELNGASLFFP |
| | | DAYRQWLDSIYDDAEMDEPEWVGNGMDKFESAECEKRFKARKVLQWAEEYSLQ |
| | | DNDETILAVTRDGEMSLPLLPYVQTSSGKQLLDGQVYEDLSHEQQYEALALNRVN |
| | | VPFTWKRSFSEVVDEDGLLWLEGKQNLDGWVWQGNSIVITYTGDEGMTRVIPANP |
| | | K* |
| Cse1/ | 16 | MNLLIDNWIPVRPRNGGKVQIINLQSLYCSRDQWRLSLPRDDMELAALALLVCIGQ |
| CasA | | IIAPAKDDVEFRHRIMNPLTEDEFQQLIAPWIDMFYLNHAEHPFMQTKGVKANDVT |
| | | PMEKLLAGVSGATNCAFVNQPGQGEALCGGCTAIALFNQANQAPGFGGGFKSGLR |
| | | GGTPVTTFVRGIDLRSTVLLNVLTLPRLQKQFPNESHTENQPTWIKPIKSNESIPASSI |
| | | GFVRGLFWQPAHIELCDPIGIGKCSCCGQESNLRYTGFLKEKFTFTVNGLWPHPHSP |
| | | CLVTVKKGEVEEKFLAFTTSAPSWTQISRVVVDKIIQNENGNRVAAVVNQFRNIAP |
| | | QSPLELIMGGYRNNQASILERRHDVLMFNQGWQQYGNVINEIVTVGLGYKTALRK |
| | | ALYTFAEGFKNKDFKGAGVSVHETAERHFYRQSELLIPDVLANVNFSQADEVIADL |
| | | RDKLHQLCEMLFNQSVAPYAHHPKLISTLALARATLYKHLRELKPQGGPSNG* |
| Cse2/ | 17 | MADEIDAMALYRAWQQLDNGSCAQIRRVSEPDELRDIPAFYRLVQPFGWENPRHQ |
| CasB | | QALLRMVFCLSAGKNVIRHQDKKSEQTTGISLGRALANSGRINERRIFQLIRADRTA |
| | | DMVQLRRLLTHAEPVLDWPLMARMLTWWGKRERQQLLEDFVLTINKNA* |
| Cas7/ | 18 | MSNFINIHVLISHSPSCLNRDDMNMQKDAIFGGKRRVRISSQSLKRAMRKSGYYAQ |
| Cse4/ | | NIGESSLRTIHLAQLRDVLRQKLGERFDQKIIDKTLALLSGKSVDEAEKISADAVTP |
| CasC | | WVVGEIAWFCEQVAKAEADNLDDKKLLKVLKEDIAAIRVNLQQGVDIALSGRMA |
| | | TSGMMTELGKVDGAMSIAHAITTHQVDSDIDWFTAVDDLQEQGSAHLGTQEFSSG |
| | | VFYRYANINLAQLQENLGGASREQALEIATHVVHMLATEVPGAKQRTYAAFNPAD |
| | | MVMVNFSDMPLSMANAFEKAVKAKDGFLQPSIQAFNQYWDRVANGYGLNGAAA |
| | | QFSLSDVDPITAQVKQMPTLEQLKSWVRNNGEA* |
| Cas5/ | 19 | MRSYLILRLAGPMQAWGQPTFEGTRPTGRFPTRSGLLGLLGACLGIQRDDTSSLQA |
| CasD | | LSESVQFAVRCDELILDDRRVSVTGLRDYHTVLGAREDYRGLKSHETIQTWREYLC |
| | | DASFTVALWLTPHATMVISELEKAVLKPRYTPYLGRRSCPLTHPLFLGTCQASDPQ |
| | | KALLNYEPVGGDIYSEESVTGHHLKFTARDEPMITLPRQFASREWYVIKGGMDVSQ* |
| Cas6/ | 20 | MYLSKVIIARAWSRDLYQLHQGLWHLFPNRPDAARDFLFHVEKRNTPEGCHVLLQ |
| Cse3/ | | SAQMPVSTAVATVIKTKQVEFQLQVGVPLYFRLRANPIKTILDNQKRLDSKGNIKR |
| CasE | | CRVPLIKEAEQIAWLQRKLGNAARVEDVHPISERPQYFSGDGKSGKIQTVCFEGVLT |
| | | INDAPALIDLVQQGIGPAKSMGCGLLSLAPL* |
| Cas3 | 21 | ATGAATGTCCTAATTATCTCACGCTGTACGAAAAATGCTCGCGTGGAAAGCTGC |
| | | CGTATTATTGATCAGTTTGCCGAACGCACTGGTGACGCAGCATGGCAAACGGTG |
| | | ATTACTCTGGAAGGCGTTAATACACTCCGCCGTTTACTGCGCAAAACCGCCAGG |
| | | CGAAATACCGCAGTGGCTTGTCACTGGCTGAAGAAGAATGGGCAAACGGAGCT |
| | | GATGTGGATTGTCGGCAATATTCGTCGCTTTAATGCTCAAGGCCGCGTTCCCAC |
| | | AAATCGAACAACGCAAATCAGCCTGGTGAACAAAGAAGAAAATCGCTGGCAGT |
| | | GCGCGGAAAGTATTGCACTGCTGGCGGCTATCGCTGGGCTATTTCATGATTTTG |
| | | GTAAATCAGGACGCAGCTTTCAGCAAAGCCTGACTCAAAAGAATACCCGCAGT |
| | | TATCAACCTTACCGACATGAATGGATCTCACTGCGGCTCTTTCAGGCGTTTGTC |
| | | GGTGATCGGCCCGACGATGTGTGGCTGGCGAAGCTTGGCGAACTCACGGCAGA |
| | | TGATGAATCTGCGATTCTTGAGCGCCTGCAAAAAGACACTCCTCAATTTTCAAA |
| | | TAGCCCGTTCAGTTCGCTGCAACCATTAGCCAAAACGGTAGGCTGGCTGATCCT |
| | | TTCCCATCATCGAATGCCGGAGAATCTTTCTCCACAGTCGGAACAATCATGTAA |
| | | AGGGTGTGAAAGTTGGCTGGAGAAGCAACTTAATGCTGACTGGAATGCCGTTA |
| | | ACCACCGCAGAGATGACTGGTCACAACAGGATTTTGCGCACGTCTGGCAGTTTC |
| | | CATCAGGAACGCCGCTTCAAAGCGCGAGCTGGCGGGAAAAAGCGCGTCAGATA |
| | | GCCCGCCGGGCAAGCAACGCGCTAGGTTTAAAGACATATGGGCAGATGGAACA |
| | | GTTGTTCCCCTTACATATGGCGCGTTTTTGTCTGATGCTTGCCGATCATTACTAT |
| | | TCCTCAAGCGATGCGCAGCAAAAATGGCAGGACCCGGATTATATCGTCTGGGC |
| | | AAATAGCGATCGCCAAAGCGGCACGCTCAAACAACGGGTAGACGAACACCTTG |
| | | TGGGCGTCGCACATCATGCGTATCTGCTTGGGCGGCAACTGATGCACACACGGG |
| | | CATCGCTTCCGGCAATTGCCCGACATAAAGGTTTTAGCCAGCGGGCCATAGATG |
| | | TGAATTATCGCTGGCAAGATAAAGCCTGGGATGTGGCGCGAGCGTTACGTGAA |
| | | CGAAGTCGTGAAGAGGGATTTTTCGGCGTCAATATGGCCTCGACGGGGTGTGGT |
| | | AAAACCTTTGCGAACGCCCGCATTATGTATGCACTTGCCGACGAGCGAGAAGG |
| | | GTGCCGCTTTACCGTTGCGCTGGGATTGCGCACGCTGACTTTACAAACCGGCGA |
| | | TGCTCTGCGCAGTCGGCTTGGGCTGGGTGAGGATGAGCTGGCGGTACTGATTGG |
| | | TTCGGCGGCAGTTAATCAACTGTGGCAACAGGAAAAAATAGACAATGATTGCG |
| | | GAAGTGCTTCGCAAGAGTCGCCGGCAGAGGAGCAACAGTTCGTTAAGTACGAA |
| | | GGCAGCCTGCATACCGGGACGCTGGAAAAATGGTTGAAGGATGACAGCAAACT |
| | | TAAGGAGTTAGTAAGCGCGCCGATTCTGGTCTCGACGATTGATCATCTTATTTC |
| | | CGCGACCGAGGGCGTGCGAGGCGGGAAACAGCTTCCGGCGATGTTGCGCCTGT |
| | | TAACCAGCGATTTAGTGCTCGACGAACCTGATGACTTTGATATTGCCGATCTTC |
| | | ATGCGGTATGTCGATTGATGAACTGGGCCGGGATGCTGGGAACGCGGGTATTA |
| | | CTTTCTTCCGCGACGTTACCGCCAGGTTTGATTCAGGCGCTGTTCGCGGCTTATC |
| | | TTGCTGGGCGAAAAATGTGGCAGGCGTCTTGTGGTATCAACGGCAGGCCCGTC |
| | | AATATATGCTGCGCCTGGTTTGATGAAAAAGATGCTGATGCCACGCAAATTTAC |
| | | GACGGACCTGGGTTTCGTGATGCTCATGCCAAATTTGTTGCACGACGAGCGGTA |
| | | ATGCTGGCTGAAAAAGAACGATTGCATTTCGGGCGTGTAGCTTCGGTTTCATCA |
| | | GCATCAGGCGCGATACAAGATGTTACCGAAAGTGTGGCCCAGACGGTGCATAC |
| | | GCAAATGCTGAAGTTGCATCAGGCGCATCGCCAGCGACACGAAAGCGGTAAAA |
| | | CCGTTTCGCTGGGCCTGGTGCGCTTCGCGAATATCAATCCACTGGTTGCGGTGA |
| | | CCAAAGCCCTGATTGTCATTCCTTCACCAGAAGATGTCTGCATTCATTACTGTGT |
| | | TTACCACTCCCGACACCCGCTGGCGGTGCGTTCAGATATTGAGAAGCGACTGGA |
| | | TCGCGCTTTTACTCGTCATAACGAACTCGATTTCTGGAACAACGAAGATATCGC |
| | | AGATGCCTTACATAACCGTCCGGAATCGCACCATCTGTTTGTGGTGTTGGGGAC |
| | | ATCGGTTATAGAAGTGGGGCGTGACTGGGATGCGGACTGGGGAATTATCGAAC |
| | | CAAGCTCCATGCGTTCGATAATTCAATTTGCCGGACGTATTCAGCGCCACCGTA |
| | | GAAATGTGCCGACCAGTGAGAATCTGGTTATTTTGCGTAGCAATATCAAATCAT |
| | | TGCAGAAAAAAGCCCCCGCATTTTGTAAGCCTGGTTTCGAAACAAAAGAACAC |
| | | TTGCTGAATAGCCACGATATTTGCGAACTGATTCCGATCGCGGACTACCGGACG |
| | | ATTAACGCCTTGCCGCGGATCCTGTCTGCCAATAATGCCAACAAACTGGCGGAG |
| | | CTAGAACATAGTCGTTTATCCGCCGAATTGATGAGCAGTAAAAGCAAACATGT |
| | | GGTGGCGGCGCAATGGTGGCGTGCACCGCTAAGCTGGAACGGCTGGATGCAGC |
| | | GCCAGACGCCATTTCGTTATTCCCCTTTGCCGGAGGCGGTGTTTTTTCTGCATAT |
| | | GGATGATGAAGAAAGCGAAGCGCGTTTCTATTCCCGCACGGGCGACAACGAGC |
| | | GTAAAGCGCAAGGGAATTTCAGGCGAGAAGAGGTGAGATGCGCCAGTGGCGTC |
| | | GTTTGCTGGGGAGTGATGGATTACCAACACGTTTTGTTGGATTTGGCTGAAAAG |
| | | CAGAGTTGTGAGATAGCAAGCGTTGGGGAAAAGTTTGCGGAAGTGAGAGTACC |
| | | CGTCAGTGAAGAAGATGCGGTCGATGACTGGCGTTATCACCCCTGGTTAGGTGT |
| | | GTTTCGCAACATGTGA |
| Cas8f/ | 22 | ATGGAAAAAACGGCGCTAATACAATTTATTACTGACTACATCGCTTCGCGAAGG |
| Csy 1 | | CAACACAAAATCGATGCCTTTGAAAAAGAAGCCGCAAAGCGAGTGGAACAGG |
| | | GCGAGGACGCCAGCGTTATCGCTCAGGAGCGGCAGGAACTCGAAGCACGCTAC |
| | | TTGCCACGTAACTGGTTAACGGATGCGGCAAAGCGTGCCGGGCAAATCAAATT |
| | | AGTCACTCATGGCGCTAAATTCAGTCATGGTGATTCTAAAGCCAGTAGCTTTTA |
| | | TCTGGAAACCTCCGCTAACGAAAGCTATTTAAACACAGCATCGTTGGCGAATGT |
| | | CGCTACTGACGCGATTGGTAACGCCGCTGCACTTGACGTAGCAAAGTTGCTACA |
| | | AACCGATGTGAACGGAGATTCACTGCTTGCCAGCCTGAAGCGTGGTGATTATCA |
| | | GGCATTAAGTACGTTTGCAGAGGATAAAGTGCAACTGGAGCTGTGGGTAGCTG |
| | | GGTTTAGCCAGGCATGGACTACTGGACGGCCTTCCAGCCATAAACTGGCGAAA |
| | | CAAATCTATTTCCCTGTTGCGGATGGTTATCACCTGTTATGCCCACTGTTTTCCA |
| | | GTTCACTGGCCCAGGCGATGTATGAAAAGCTAACCGCCGGGCGCTTTAGTGAG |
| | | GAATCGAAAGCCATTCGCGATGCCCGCAAAGCAGGAAAATGGCACTCCCAGCC |
| | | AGATATCCGGTTTCCTAATCTTGCCGAAATGCATTTCGGCGGCACAAAACCGCA |
| | | GAATATTTCTTTGCTAAACAGTGTGCGTGGTGGGCGCGTCTGGCTGTTGCCGTC |
| | | AATGCCGCCTGCCTGGGGGCCGCTGGATCGCGCACCACAAAAAATGCACAGTA |
| | | TTTTTGCCTTACGCGGCGATTTCAATAGGGCGGCTTCAGGCATCATAGCCCGGA |
| | | TGACTTACCTGCTGAAGGTTGACACAAATAATGTTCATATCCGAACTGTGCGGG |
| | | CGAAATATATTGATGAGTTGATCGATTTGCTGTTCATGCAGGCATCCGCTTTTCA |
| | | GCAAGAAAAATGGCAGGGATGGTCCGCACAATCACCAGACTTACCTCGCCACC |
| | | AACAACTTTGGCTGGACCCGTGGCGAAGCCTCTCAGATGAGACTTTCAAACAG |
| | | GAGCGTGAGAAAAACGACTGGCAAGTAACTGTCGCCGATGATTTCGCACGCTG |
| | | GCTGAACTATCGCCTGAAAAAATCCAGTTTTGACGTAGGCGCTGTCGAACAAA |
| | | AAGAGTGGCGCAGCCAGTCTCTGTTCGCTCAACGGATGCGTGAAATGGAAGCT |
| | | GTTTTGCAGGAGGCGCTGAAATGA |
| Cas5/ | 23 | ATGAGTTATCTGCTCTTGTTACCCCATATACGCATCGAAAACGCTAACGCTGTC |
| Csy2 | | TCCGGGTTGACCTGGGGATTCCCGTCGATGACCCATTTTCTCGGTTATGTCCATG |
| | | CGCTTTCTCGCAAAGTCGTGGATGAATTTGGCGTAAGTTTTGATGGTTGTGCGG |
| | | TAGTAAGCCATGAACAGCATATTCAGGCGTACAGCTCTGGGCGCGATTTTCAGT |
| | | TCGCTCTCACCCGTAACCCATTAACCAGAGAAGGTAAAACCGCCTCGTTCAATG |
| | | AAGAGGGCCGCATGCACTTGACGGTTTCTCTACTGATTGAATGTAATGGCGAAA |
| | | TCACCAATGGTGAATATGGCAGAAAAGTTCTTTGTGACTATCTGAAAATGCTTT |
| | | GCCAGAGCCACAAACTGGCGGGCGGCAGCATTGTCAGTATGCGCGATCCGCAG |
| | | CTTTTTCATGCGCCTGAAGATGAAAAGCAACTGCGTAAAATCGTCTGGCGGTTA |
| | | ATGCCGGGTTATGCGCTTTACGATCGCAGTGAATGGCTGGCTGAACATCACCAA |
| | | CAACATCCCGACATCTCGTTGCTGGATGCCTGGCTTGATTTCGCCGCGATTAAA |
| | | TATCAGGCCGAATCTCCGGCTGAAGACAACAGCGCAAAATGGGTATACCAGCC |
| | | GAAGCCAATGCCTGGTTTCCTGGTACCGTTAATGTGTGGCTACCAACGCATTTC |
| | | TCCTGTTTACGCACCCGGTGAAGTTGAAAATGCGCGCGACACCGTGACGCCATT |
| | | TGCCTTTGCCGAAGCGGTATATGGCATTGGCGAATGGCGCGGATTACATCGCAC |
| | | TACAGACTTGCAAGCGCTGATGTGGCGTTACCACACAACGGACACTGGCTACTA |
| | | TTGTTCGGCAACGCCAGTTGTTGATGATTTTACCTTTAATGAATATGACGATTTA |
| | | GAATAA |
| Cas7/ | 24 | ATGGCAAAGGCTCCTGTAGCAATAAAAACGGCGTCGGTACTGGCATTTGAGCG |
| Csy3 | | CAAACTGGCAACCTCTGACGCGGTAATGTACGCAGGTAACTGGCAAGGAAATG |
| | | AATGGCAACCCATTGAGATTCAGGAGAAAGCGGTCCGCGGAACCATCTCTAAT |
| | | CGCCTGAAAAATGCCATTGTCAGCGATCCCACAAAACTGGATGCTGAAATCCA |
| | | AAAAGCCAACCTGCAACGGGTGGATGTGGCTTCGCTTCCGGTAGATACGGATA |
| | | CGTTAAAGGTGGTGTTTACGCTGCGTGTGCTGGGTAATTTATCCACACCGTCAG |
| | | TATGTAATGATATGGCTTACCAGGAGGCGCTTTCCGGGGTCATTGAAGGTTACA |
| | | TCAGCGAACATGGCTTTAAAGAGCTGGCGCTTCGCTATGCGCTGAATTTGGCAA |
| | | ATGGTCGTTTTCTGTGGCGCAACCGCATTGGTGCCGAACAGATCCAGGTGAAAG |
| | | TGACTGCTAACGAGACTTCATGGACTTTTAACAGCCATGACTTCTCTTTTACGTCA |
| | | ATTTGACCAGGGACAGGCAGCGGTCACGCAGCTGGCGGCCATTATAGAGCAAG |
| | | GATTAAGCGCAAAAGAGTGGGTGATGCTTACCGTTGAAGCGCAGGTTCGTCTTG |
| | | GTGCCGGGCAAGAAGTTTTTCCGTCGCAGGAGCTGGTACTCGACAGCAACAGT |
| | | AGTAAAAGTCGCGTGTTATACCAGGTGGCAGGCATTGCCGGTATTCACTCTCAG |
| | | AAGATTGGCAATGCTTTACGTACCATTGATACCTGGCATCCAAAGGTTGATGAA |
| | | TTGGGGGCTATAGCCGTGGAACCTTACGGTTCTGTAACCAGTCGCGGTGTGGCC |
| | | TGCCGTCAACCGAAAGAAAAACTCGACTTTTATACCTTACTGGACAATTGGGTG |
| | | ACTAAAGGGATGAAACCAGACGTTAAGCAGCAGCACTACGTGATGGCTGTGCT |
| | | GATCCGTGGCGGTGTCTTTGGTGAGAAATCGGAATAA |
| Cas6f | 25 | ATGGATCACTACCTTGAGATCCGCGTTCTGCCAGATCCTGAATTTTCGAGCGAA |
| | | ATGTTGATGGCGGCGTTATTTGCAAAACTGCATCGGGTATTAGGTGCAAGAGGG |
| | | CAAGGCGATATCGGCGTGAGTTTCCCGGACGTAAATGTTATGCCTGGAACGCAT |
| | | TTACGCTTGCATGGTAGCGCCCAGGCATTGCAAGAACTTGAAGCCTCGACGTGG |
| | | CGTAAAGGGCTGACAGATTACTGCCAATGTTCGCCTGTCACGCCAGTTCCTGAA |
| | | ATTAAAGGCTGGCGAGTCGTAAGTCGTGTACAGGTAAAAAGTAACCCTCAGCG |
| | | TCTGTTAAGGCGTTCAGTGAAAAAAGGCTGGCTAACTGAAGAACAGGCAATTG |
| | | AACGACTGGCGACGCAGGCTGAACAGCGGACCGATTTACCCTTTCTTAATATGA |
| | | AGAGTTTGTCGAGTCAGCAACAGTTCAAGTTGTTTATCCGTCATGGTGATTTGC |
| | | TCAAAGAACCGGTTAAAGGGGAATTTAGTAGTTATGGATTAAGCGCAACCGCC |
| | | ACTATTCCCTGGTTTTGA |
| Cas3 | 26 | MNVLIISRCTKNARVESCRIIDQFAERTGDAAWQTVITLEGVNTLRRLLRKTARRNT |
| | | AVACHWLKKNGQTELMWIVGNIRRFNAQGRVPTNRTTQISLVNKEENRWQCAESI |
| | | ALLAAIAGLFHDFGKSGRSFQQSLTQKNTRSYQPYRHEWISLRLFQAFVGDRPDDV |
| | | WLAKLGELTADDESAILERLQKDTPQFSNSPFSSLQPLAKTVGWLILSHHRMPENLS |
| | | PQSEQSCKGCESWLEKQLNADWNAVNHRRDDWSQQDFAHVWQFPSGTPLQSAS |
| | | WREKARQIARRASNALGLKTYGQMEQLFPLHMARFCLMLADHYYSSSDAQQKW |
| | | QDPDYIVWANSDRQSGTLKQRVDEHLVGVAHHAYLLGRQLMHTRASLPALARHK |
| | | GFSQRAIDVNYRWQDKAWDVARALRERSREEGFFGVNMASTGCGKTFANARIMY |
| | | ALADEREGCRFTVALGLRTLTLQTGDALRSRLGLGEDELAVLIGSAAVNQLWQQE |
| | | KIDNDCGSASQESPAEEQQFVKYEGSLHTGTLEKWLKDDSKLKELVSAPILVSTIDH |
| | | LISATEGVRGGKQLPAMLRLLTSDLVLDEPDDFDIADLHAVCRLMNWAGMLGTRV |
| | | LLSSATLPPGLIQALFAAYLAGRKMWQASCGINGRPVNICCAWFDEKDADATQIYD |
| | | GPGFRDAHAKFVARRAVMLAEKERLHFGRVASVSSASGAIQDVTESVAQTVHTQ |
| | | MLKLHQAHRQRHESGKTVSLGLVRFANINPLVAVTKALIVIPSPEDVCIHYCVYHS |
| | | RHPLAVRSDIEKRLDRAFTRHNELDFWNNEDIADALHNRPESHHLFVVLGTSVIEV |
| | | GRDWDADWGIIEPSSMRSIIQFAGRIQRHRRNVPTSENLVILRSNIKSLQKKAPAFCK |
| | | PGFETKEHLLNSHDICELIPIADYRTINALPRILSANNANKLAELEHSRLSAELMSSKS |
| | | KHVVAAQWWRAPLSWNGWMQRQTPFRYSPLPEAVFFLHMDDEESEARFYSRTGD |
| | | NERKAQGNFRREEVRCASGVVCWGVMDYQHVLLDLAEKQSCEIASVGEKFAEVR |
| | | VPVSEEDAVDDWRYHPWLGVFRNM* |
| Cas8f/ | 27 | MEKTALIQFITDYIASRRQHKIDAFEKEAAKRVEQGEDASVIAQERQELEARYLPRN |
| Csy 1 | | WLTDAAKRAGQIKLVTHGAKFSHGDSKASSFYLETSANESYLNTASLANVATDAI |
| | | GNAAALDVAKLLQTDVNGDSLLASLKRGDYQALSTFAEDKVQLELWVAGFSQAW |
| | | TTGRPSSHKLAKQIYFPVADGYHLLCPLFSSSLAQAMYEKLTAGRFSEESKAIRDAR |
| | | KAGKWHSQPDIRFPNLAEMHFGGTKPQNISLLNSVRGGRVWLLPSMPPAWGPLDR |
| | | APQKMHSIFALRGDFNRAASGIIARMTYLLKVDINNVHIRTVRAKYIDELIDLLFMQ |
| | | ASAFQQEKWQGWSAQSPDLPRHQQLWLDPWRSLSDETFKQEREKNDWQVTVAD |
| | | DFARWLNYRLKKSSFDVGAVEQKEWRSQSLFAQRMREMEAVLQEALK* |
| Cas5/ | 28 | MSYLLLLPHIRIENANAVSGLTWGFPSMTHFLGYVHALSRKVVDEFGVSFDGCAV |
| Csy 2 | | VSHEQHIQAYSSGRDFQFALTRNPLTREGKTASFNEEGRMHLTVSLLIECNGEITNG |
| | | EYGRKVLCDYLKMLCQSHKLAGGSIVSMRDPQLFHAPEDEKQLRKIVWRLMPGY |
| | | ALYDRSEWLAEHHQQHPDISLLDAWLDFAAIKYQAESPAEDNSAKWVYQPKPMP |
| | | GFLVPLMCGYQRISPVYAPGEVENARDTVTPFAFAEAVYGIGEWRGLHRTTDLQA |
| | | LMWRYHTTDTGYYCSATPVVDDFTFNEYDDLE* |
| Cas7/ | 29 | MAKAPVAIKTASVLAFERKLATSDAVMYAGNWQGNEWQPIEIQEKAVRGTISNRL |
| Csy 3 | | KNAIVSDPTKLDAEIQKANLQRVDVASLPVDTDTLKVVFTLRVLGNLSTPSVCNDM |
| | | AYQEALSGVIEGYISEHGFKELALRYALNLANGRFLWRNRIGAEQIQVKVTANETS |
| | | WTFNSHDFSLRQFDQGQAAVTQLAAIIEQGLSAKEWVMLTVEAQVRLGAGQEVFP |
| | | SQELVLDSNSSKSRVLYQVAGIAGIHSQKIGNALRTIDTWHPKVDELGAIAVEPYGS |
| | | VTSRGVACRQPKEKLDFYTLLDNWVTKGMKPDVKQQHYVMAVLIRGGVFGEKSE* |
| Cas6f | 30 | MDHYLEIRVLPDPEFSSEMLMAALFAKLHRVLGARGQGDIGVSFPDVNVMPGTHL |
| | | RLHGSAQALQELEASTWRKGLTDYCQCSPVTPVPEIKGWRVVSRVQVKSNPQRLL |
| | | RRSVKKGWLTEEQAIERLATQAEQRTDLPFLNMKSLSSQQQFKLFIRHGDLLKEPV |
| | | KGEFSSYGLSATATIPWF* |

### Bacteriophage

In some embodiments, the bacteriophage disclosed herein is an obligate lytic bacteriophage. In some embodiments, the bacteriophage disclosed herein is not a lytic bacteriophage. In some embodiments, the bacteriophage is a temperate bacteriophage. In some embodiments, the bacteriophage is a temperate bacteriophage with a lysogeny gene removed, replaced, or inactivated, thereby rendering the bacteriophage lytic. In some embodiments, the bacteriophage is not a temperate bacteriophage. In some embodiments, the bacteriophage comprises a CRISPR-Cas system. In some embodiments, the bacteriophage does not comprise a CRISPR-Cas system.

In some embodiments, the bacteriophages include, but are not limited to, T4, T7, T7m, M13, p0046-9, p0033s-6, p0071-16, p0033L-10, p00ex-2, p0031-8, p004k-5, p0045-9, p0078-4, p00dd-1, p00E8-3, or p00Jc-2. In some embodiments, the phage is crT7m. In some embodiments, the phage is crT4. In some embodiments, the phage is crT7. In some embodiments, the phage is crM13. In some embodiments, the phage is p0046-9. In some embodiments, the phage is p0033s-6. In some embodiments, the phage is p0071-16. In some embodiments, the phage is p0033L-10. In some embodiments, the phage is p00ex-2. In some embodiments, the phage is p0031-8. In some embodiments, the phage is p004k-5. In some embodiments, the phage is p0045-9. In some embodiments, the phage is p0078-4. In some embodiments, the phage is p00dd-1. In some embodiments, the phage is p00E8-3. In some embodiments, the phage is p00Jc-2. In some embodiments, the bacteriophage is T4 E. coli bacteriophage. In some embodiments, the bacteriophage is T7 E. coli bacteriophage. In some embodiments, the bacteriophage is T7m E. coli bacteriophage. In some embodiments, the bacteriophage is M13 E. coli bacteriophage. In some embodiments, the bacteriophage is p0046-9 E. coli bacteriophage. In some embodiments, the bacteriophage is p0033s-6 E. coli bacteriophage. In some embodiments, the bacteriophage is p0071-16 E. coli bacteriophage. In some embodiments, the bacteriophage is p0033L-10 E. coli bacteriophage. In some embodiments, the bacteriophage is p00ex-2 E. coli bacteriophage. In some embodiments, the bacteriophage is p0031-8 E. coli bacteriophage. In some embodiments, the bacteriophage is p004k-5 E. coli bacteriophage. In some embodiments, the phage is p0045-9 E. coli bacteriophage. In some embodiments, the phage is p0078-4 E. coli bacteriophage. In some embodiments, the phage is p00dd-1 E. coli bacteriophage. In some embodiments, the phage is p00E8-3 E. coli bacteriophage. In some embodiments, the phage is p00Jc-2 E. coli bacteriophage. In some embodiments, the bacteriophage is and unmodified E. coli phage. In some embodiments, the bacteriophage is p004k (PTA-127149) phage. In some embodiments, the bacteriophage is p00c0 (PTA-127143). In some embodiments, the bacteriophage is p00ex (PTA-127145). In some embodiments, the bacteriophage is p00jc (PTA-127147). In some embodiments, the bacteriophage is p00ke (PTA-127148). In some embodiments, the bacteriophage is p5516 (PTA-127151).

In some embodiments, the bacteriophage is an unmodified Bacteroides phage, e.g., a wild type Bacteroides phage. In some embodiments, the bacteriophage is p3854-40-8. In some embodiments, the bacteriophage is p3855-56-3. In some embodiments, the bacteriophage is p4075. In some embodiments, the bacteriophage is p4076. In some embodiments, the bacteriophage is p4077. In some embodiments, the bacteriophage is p4078. In some embodiments, the bacteriophage is p4079. In some embodiments, the bacteriophage is p4082. In some embodiments, the bacteriophage is p4083. In some embodiments, the bacteriophage is p4084. In some embodiments, the bacteriophage is p4085. In some embodiments, the bacteriophage is p4087. In some embodiments, the bacteriophage is p4088. In some embodiments, the bacteriophage is p4090. In some embodiments, the bacteriophage is p4092. In some embodiments, the bacteriophage is p4093. In some embodiments, the bacteriophage is p4094. In some embodiments, the bacteriophage is p5097. In some embodiments, the bacteriophage is p5496. In some embodiments, the bacteriophage is p5497. In some embodiments, the bacteriophage is p5499. In some embodiments, the bacteriophage is p5501. In some embodiments, the bacteriophage is p5503. In some embodiments, the bacteriophage is p5505. In some embodiments, the bacteriophage is p5506. In some embodiments, the bacteriophage is p5507. In some embodiments, the bacteriophage is p5508. In some embodiments, the bacteriophage is p5509. In some embodiments, the bacteriophage is p5511. In some embodiments, the bacteriophage is p5512.

In some embodiments, the bacteriophage is an unmodified Enterococcus phage, e.g., a wild type Enterococcus phage. In some embodiments, the bacteriophage is p006008. In some embodiments, the bacteriophage is p006009. In some embodiments, the bacteriophage is p006010. In some embodiments, the bacteriophage is p006012. In some embodiments, the bacteriophage is p006013. In some embodiments, the bacteriophage is p006016. In some embodiments, the bacteriophage is p006018. In some embodiments, the bacteriophage is p006071. In some embodiments, the bacteriophage is p006072. In some embodiments, the bacteriophage is p006098. In some embodiments, the bacteriophage is p006099. In some embodiments, the bacteriophage is p006128. In some embodiments, the bacteriophage is p006129. In some embodiments, the bacteriophage is p5852. In some embodiments, the bacteriophage is p5853.

In some embodiments, the bacteriophage infects a bacteria that contributes to the pathogenesis of a disease. In some embodiments, the bacteriophage infects an E. coli bacteria, that causes a disease, a condition, a syndrome, or a discomfort in a human subject. In some embodiments, the bacteriophage infects a bacteria of the genus Bacteroides, e.g., B. fragilis, that causes a disease, a condition, a syndrome or a discomfort in a human subject. In some embodiments, the bacteriophage infects a bacteria of the genus Enterococcus, e.g., E. faecium, that causes a disease, a condition, a syndrome or a discomfort in a human subject. In some embodiments, the bacteria causes an enteric disease. In some embodiments, the bacteria causes an inflammatory disease. In some embodiments, the bacteria causes an immunomodulation. In some embodiments, the bacteria causes an autoimmune disease. In some embodiments, the bacteria causes a cancer. In some embodiments the cancer is colorectal cancer.

In some embodiments, bacteriophages of interest are obtained from environmental sources or from commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

In some embodiments, a bacteriophage disclosed herein is lytic, lysogenic or temperate. In some embodiment the bacteriophage following modification can replicate in a target bacteria. In some embodiment the bacteriophage following modification can lyse a target bacteria.

In some embodiments, the bacteriophage can differentially target a first microbe compared to a second microbe. In some embodiments, the bacteriophage, upon targeting, results in killing of a first microbe, but does not kill a second microbe, as is described herein. In some embodiments, the bacteriophage targets and kills a first microbe preferentially over a second microbe, wherein the first microbe and the second microbe may be of the same genus, may be of the same species, but may be functionally non-identically in a given human or a non-human subject. In some embodiments, the bacteriophage targets and kills a first microbe more efficiently than a second microbe. In some embodiments, the bacteriophage targets and kills a first microbe more efficiently than a second microbe in a given subject. In some embodiments, the bacteriophage targets and kills a first microbe more efficiently, e.g. at a higher rate than a second microbe. In some embodiments, the bacteriophage targets the population of a first microbe and does not target the population of a second microbe. In some embodiments, the bacteriophage targets the population of a first microbe and does not target the population of a second microbe within a given microflora of a subject. In some embodiments the bacteriophage targets a population of the first microbe and a population of the second microbe but effectively reduces the population of the first microbe e.g., by about 20%, 30%, 40%, or 50% of an initial population, or more; and at the same time, minimally reduces the population of the second microbe, e.g. by about 15%, 12%, 10%, or less than 10% of an initial population. In some embodiments, the bacteriophage prevents the growth of the first population of microbe, and does not prevent the growth of the second population of microbe. In some embodiments, the bacteriophage selectively restricts the expansion of a microbial population. In some embodiments, the bacteriophage selectively restricts or prevents the growth of the population of the first microbe with greater efficiency, e.g. by greater than about 20%, 30%, 40%, 50%, 60%, 70%, 80%, 90% or more than the efficiency with which it restricts or prevents the growth of the population of second microbe. In some embodiments, the bacteriophage does not allow the population of the first microbe to rise, grow or grow back. In some embodiments, the bacteriophage allows long term effect as a bacteriostatic or bacteriolytic to the first microbe relative to the second microbe, wherein the term bacteriostatic indicates lack or reduction in growth or expansion of a population of bacteria, e.g., the population of the first microbe; and bacteriolytic is associated with reduction or killing of a population of bacteria, e.g., the population of the first microbe.

In some embodiments, the bacteriophage can equally target a first microbe and a second microbe. In some embodiments, the bacteriophage, upon targeting, results in killing of both a first microbe and a second microbe, as is described herein. In some embodiments, the bacteriophage targets the population of a first microbe and the population of a second microbe. In some embodiments, the bacteriophage targets the population of a first microbe and the population of a second microbe within a given microflora of a subject. In some embodiments, the bacteriophage prevents the growth of the first population of microbe and the growth of the second population of microbe. In some embodiments, the bacteriophage does not allow the population of the first microbe or the second microbe to rise, grow or grow back. In some embodiments, the bacteriophage allows long term effect as a bacteriostatic or bacteriolytic to the first microbe and to the second microbe, wherein the term bacteriostatic indicates lack or reduction in growth or expansion of a population of bacteria.

### Insertion Sites

In some embodiments, the insertion of the nucleic acid sequence into a bacteriophage does not disrupt the lytic or replicative activity of the bacteriophage. In some embodiments, the insertion of the nucleic acid sequence into a bacteriophage preserves the lytic activity of the bacteriophage. In some embodiments, the nucleic acid sequence is inserted into the bacteriophage genome. In some embodiments, the nucleic acid sequence is inserted into the bacteriophage genome at a transcription terminator site at the end of an operon of interest. In some embodiments, the nucleic acid sequence is inserted into the bacteriophage genome as a replacement for one or more removed non-essential genes. In some embodiments, the nucleic acid sequence is inserted into the bacteriophage genome as a replacement for one or more removed lysogenic genes. In some embodiments, the replacement of non-essential and/or lysogenic genes with the nucleic acid sequence does not affect the lytic activity of the bacteriophage. In some embodiments, the replacement of non-essential and/or lysogenic genes with the nucleic acid sequence preserves the lytic activity of the bacteriophage. In some embodiments, the replacement of non-essential and/or lysogenic genes with the nucleic acid sequence enhances the lytic activity of the bacteriophage. In some embodiments, the replacement of non-essential and/or lysogenic genes with the nucleic acid sequence renders a lysogenic bacteriophage lytic.

In some embodiments, the nucleic acid sequence is introduced into the bacteriophage genome at a first location while one or more non-essential and/or lysogenic genes are separately removed and/or inactivated from the bacteriophage genome at a separate location. In some embodiments, the nucleic acid sequence is introduced into the bacteriophage at a first location while one or more non-essential and/or lysogenic genes are separately removed and/or inactivated from the bacteriophage genome at multiple separate locations. In some embodiments, the removal and/or inactivation of one or more non-essential and/or lysogenic genes does not affect the lytic activity of the bacteriophage. In some embodiments, the removal and/or inactivation of one or more non-essential and/or lysogenic genes preserves the lytic activity of the bacteriophage. In some embodiments, the removal of one or more non-essential and/or lysogenic genes renders a lysogenic bacteriophage into a lytic bacteriophage.

In some embodiments, the bacteriophage is a temperate bacteriophage which has been rendered lytic by any of the aforementioned means. In some embodiments, a temperate bacteriophage is rendered lytic by the removal, replacement, or inactivation of one or more lysogenic genes. In some embodiments, the lytic activity of the bacteriophage is due to the removal, replacement, or inactivation of at least one lysogeny gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, a temperate bacteriophage is rendered lytic by the insertion of one or more lytic genes. In some embodiments, a temperate bacteriophage is rendered lytic by the insertion of one or more genes that contribute to the induction of a lytic cycle. In some embodiments, a temperate bacteriophage is rendered lytic by altering the expression of one or more genes that contribute to the induction of a lytic cycle. In some embodiments, a temperate bacteriophage phenotypically changes from a lysogenic bacteriophage to a lytic bacteriophage. In some embodiments, a temperate bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI). In some embodiments, a temperate bacteriophage that is rendered lytic is prevented from reverting to lysogenic state. In some embodiments, a temperate bacteriophage that is rendered lytic is prevented from reverting back to lysogenic state by way of introducing an additional CRISPR array. In some embodiments, a temperate bacteriophage that is rendered lytic is prevented from reverting back to lysogenic state by way of introducing an additional anti-microbial agent. In some embodiments, the bacteriophage does not confer any new properties onto the target bacterium beyond cellular death cause by lytic activity of the bacteriophage and/or the activity of a first or second CRISPR array.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

### Non-Essential Gene

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle. In some embodiments, the non-essential gene to be removed and/or replaced from the bacteriophage is the hoc gene from a T4 E. coli bacteriophage. In some embodiments, the non-essential gene to be removed and/or replaced include gp0.7, gp4.3, gp4.5, gp4.7, or any combination thereof from a T7 E. coli bacteriophage. In some embodiments, the non-essential gene to be removed and/or replaced is gp0.6, gp0.65, gp0.7, gp4.3, gp4.5, or any combination thereof from a T7m E. coli bacteriophage.

### Phage Cocktails

Also disclosed herein, in certain embodiments, are compositions comprising a plurality of bacteriophages disclosed herein. In some embodiments, the plurality of bacteriophages used together targets the same or different target bacterium within a sample or subject. In some embodiments, the bacteriophages used together comprises any combination of bacteriophages disclosed herein.

In some embodiments, the composition comprises at least two, at least three, at least four, at least five, at least six, at least seven, at least eight, at least nine, at least ten, at least eleven, at least twelve, at least thirteen, at least fourteen, or at least fifteen bacteriophages.

### Methods of Use

Disclosed herein, in certain embodiments, are methods of killing a target bacterium comprising introducing into a target bacterium (a) any of the bacteriophages disclosed herein, (b) any of the compositions disclosed herein, or (c) any of the pharmaceutical compositions disclosed herein. In some embodiments, the bacteriophages or compositions disclosed herein comprise an exogenous antimicrobial agent. In some embodiments, the exogenous antimicrobial agent targets a first microbial population to allow a second microbial population to thrive. In some embodiments, allowing the second microbial population to thrive comprises the second microbial population replicating at an increased rate compared to the first microbial population. In some embodiments, the proportion of the microbiome that is the second microbial population increases following introduction of the antimicrobial agent. In some embodiments, the proportion of the microbiome that is the second microbial population relative to the other microbial species in the microbiome increases by at least about 10%, 20%, 30% 40, %, 50%, 60%, 70%, 80%, 90%, 100%, 120%, 140%, 160%, 180%, 200%, or more than 200%.

In some embodiments, the rate of growth of the second microbial population increased following introduction of the antimicrobial agent when compared to the rate of growth of the first microbial population. In some embodiments, the rate of growth increases by at least about 10%, 20%, 30%, 40%, 50%, 60%, 70%, 80%, 90% 100% 120%, 140%, 160%, 180%, 200%, or more than 200%. In some embodiments, the rate at which the first microbial population is killed by the exogenous antimicrobial agent is greater than the rate at which the second microbial population is killed by the exogenous antimicrobial agent. In some embodiments, the rate at which the first microbial population is killed by the exogenous antimicrobial agent is greater than the rate at which the second microbial population is killed by the exogenous antimicrobial agent is greater by at least about 10%, 20%, 30%40, %, 50%, 60%, 70%, 80%, 90%, 100%, 120%, 140%, 160%, 180%, 200%, or more than 200%.

Targeting Adherent-Invasive E. coli (AIEC)

In some embodiments, provided herein are recombinant bacteriophage comprising an exogeneous antimicrobial agent specific for AIEC, e.g., as compared to a commensal E. coli. Non-limiting example AIEC strains are provided in Table 2. Non-limiting example commensal E. coli are provided in Table 2.

Non-limiting example recombinant bacteriophage specific for AIEC are described herein, e.g., bacteriophage including an antimicrobial agent comprising a CRISPR system. In some cases, the CRISPR system comprises a CRISPR array comprising a spacer sequence at least 80% identical to any one of SEQ ID NOS: 37-39. In some cases, the CRISPR system comprises a CRISPR array comprising a spacer sequence at least 80% identical to at least 10 contiguous nucleobases of a target DNA of Table 2. In some embodiments, the exogeneous antimicrobial agent is specific for the AIEC by targeting a target DNA sequence that is present in the AIEC and not present in commensal E. coli.

In some cases, the CRISPR system comprises a nucleic acid encoding a CRISPR nuclease. As an example, the CRISPR nuclease comprises Cas3, Cas3′ and Cas3″, Cpf1, or Cas9. In some cases, the CRISPR system comprises a Type I CRISPR-Cas system, a Type II CRISPR-Cas system, or a Type V CRISPR-Cas system. As an example, the CRISPR system comprises the Type I CRISPR-Cas system. Non-limiting example Type I CRISPR-Cas systems include a Type I-A CRISPR-Cas system, a Type I-B CRISPR-Cas system, a Type I-C CRISPR-Cas system, a Type I-D CRISPR-Cas system, a Type I-E CRISPR-Cas system, and a Type I-F CRISPR-Cas system. In some cases, the Type I CRISPR-Cas system is an E. coli Type I-F system (e.g., ECIF). In some cases, the Type I CRISPR-Cas system is an E. coli Type I-E system (e.g., ECIE). In some cases, the Type I CRISPR-Cas system is a P. aeruginosa Type 1-C system (e.g., PAIC). In some cases, the CRISPR nuclease comprises a Type V nuclease, e.g., a CRISPR-Cpf1 system. In some embodiments, the recombinant bacteriophage is prepared by a method comprising introducing into a first bacteriophage the exogeneous antimicrobial agent.

Further provided are methods of killing an AIEC with the recombinant bacteriophage. Such methods include methods of selectively killing AIEC as compared to commensal E. coli in a population of bacteria comprising the AIEC and the commensal E. coli. For example, the method comprises combining with the population of bacteria the recombinant bacteriophage, where the recombinant bacteriophage selectively kills the AIEC as compared to the commensal E. coli.

Further provided are methods of treating a disease or condition in a subject related to AIEC, the method comprising administering to the subject the recombinant bacteriophage. In some cases, the disease or condition is inflammatory bowel disease, or any other condition associated with AIEC, e.g., as noted in Table 1 and/or 2.

Targeting pks+E. coli (PKS+)

In some embodiments, provided herein are recombinant bacteriophage comprising an exogeneous antimicrobial agent specific for pks+E. coli, e.g., as compared to a commensal E. coli. Non-limiting example pks+E. coli strains are provided in Table 2. Non-limiting example commensal E. coli are provided in Table 2.

Non-limiting example recombinant bacteriophage specific for pks+E. coli are described herein, e.g., bacteriophage including an antimicrobial agent comprising a CRISPR system. In some cases, the CRISPR system comprises a CRISPR array comprising a spacer sequence at least 80% identical to any one of SEQ ID NOS: 40-42. In some cases, the CRISPR system comprises a CRISPR array comprising a spacer sequence at least 80% identical to at least 10 contiguous nucleobases of a target DNA of Table 2. In some embodiments, the exogeneous antimicrobial agent is specific for the pks+E. coli by targeting a target DNA sequence that is present in the pks+E. coli and not present in commensal E. coli.

In some cases, the CRISPR system comprises a nucleic acid encoding a CRISPR nuclease. As an example, the CRISPR nuclease comprises Cas3, Cas3′ and Cas3″, Cpf1, or Cas9. In some cases, the CRISPR system comprises a Type I CRISPR-Cas system, a Type II CRISPR-Cas system, or a Type V CRISPR-Cas system. As an example, the CRISPR system comprises the Type I CRISPR-Cas system. Non-limiting example Type I CRISPR-Cas systems include a Type I-A CRISPR-Cas system, a Type I-B CRISPR-Cas system, a Type I-C CRISPR-Cas system, a Type I-D CRISPR-Cas system, a Type I-E CRISPR-Cas system, and a Type I-F CRISPR-Cas system. In some cases, the Type I CRISPR-Cas system is an E. coli Type I-F system (e.g., ECIF). In some cases, the Type I CRISPR-Cas system is an E. coli Type I-E system (e.g., ECIE). In some cases, the Type I CRISPR-Cas system is a P. aeruginosa Type 1-C system (e.g., PAIC). In some cases, the CRISPR nuclease comprises a Type V nuclease, e.g., a CRISPR-Cpf1 system. In some embodiments, the recombinant bacteriophage is prepared by a method comprising introducing into a first bacteriophage the exogeneous antimicrobial agent.

Further provided are methods of killing a pks+E. coli with the recombinant bacteriophage. Such methods include methods of selectively killing pks+E. coli as compared to commensal E. coli in a population of bacteria comprising the pks+E. coli and the commensal E. coli. For example, the method comprises combining with the population of bacteria the recombinant bacteriophage, where the recombinant bacteriophage selectively kills the pks+E. coli as compared to the commensal E. coli.

Further provided are methods of treating a disease or condition in a subject related to pks+E. coli, the method comprising administering to the subject the recombinant bacteriophage. In some cases, the disease or condition is inflammatory bowel disease, colon cancer, or any other condition associated with pks+E. coli, e.g., as noted in Table 1 and/or 2.

Targeting Enterotoxigenic Bacteroides

In some embodiments, provided herein are recombinant bacteriophage comprising an exogeneous antimicrobial agent specific for an enterotoxigenic Bacteroides, e.g., as compared to a commensal Bacteroides. Bacteroides includes B. fragilis. Further non-limiting example enterotoxigenic Bacteroides strains are provided in Table 2. Non-limiting example commensal E. Bacteroides are provided in Table 2.

Non-limiting example recombinant bacteriophage specific for enterotoxigenic Bacteroides are described herein, e.g., bacteriophage including an antimicrobial agent comprising a CRISPR system. In some cases, the CRISPR system comprises a CRISPR array comprising a spacer sequence at least 80% identical to any one of SEQ ID NOS: 43-45. In some cases, the CRISPR system comprises a CRISPR array comprising a spacer sequence at least 80% identical to at least 10 contiguous nucleobases of a target DNA of Table 2. In some embodiments, the exogeneous antimicrobial agent is specific for the enterotoxigenic Bacteroides by targeting a target DNA sequence that is present in the enterotoxigenic Bacteroides and not present in commensal Bacteroides.

In some cases, the CRISPR system comprises a nucleic acid encoding a CRISPR nuclease. As an example, the CRISPR nuclease comprises Cas3, Cas3′ and Cas3″, Cpf1, or Cas9. In some cases, the CRISPR system comprises a Type I CRISPR-Cas system, a Type II CRISPR-Cas system, or a Type V CRISPR-Cas system. As an example, the CRISPR system comprises the Type I CRISPR-Cas system. Non-limiting example Type I CRISPR-Cas systems include a Type I-A CRISPR-Cas system, a Type I-B CRISPR-Cas system, a Type I-C CRISPR-Cas system, a Type I-D CRISPR-Cas system, a Type I-E CRISPR-Cas system, and a Type I-F CRISPR-Cas system. In some cases, the Type I CRISPR-Cas system is an E. coli Type I-F system (e.g., ECIF). In some cases, the Type I CRISPR-Cas system is an E. coli Type I-E system (e.g., ECIE). In some cases, the Type I CRISPR-Cas system is a P. aeruginosa Type 1-C system (e.g., PAIC). In some cases, the CRISPR nuclease comprises a Type V nuclease, e.g., a CRISPR-Cpf1 system. In some embodiments, the recombinant bacteriophage is prepared by a method comprising introducing into a first bacteriophage the exogeneous antimicrobial agent.

Further provided are methods of killing an enterotoxigenic Bacteroides with the recombinant bacteriophage. Such methods include methods of selectively killing enterotoxigenic Bacteroides as compared to commensal Bacteroides in a population of bacteria comprising the enterotoxigenic Bacteroides and the commensal Bacteroides. For example, the method comprises combining with the population of bacteria the recombinant bacteriophage, where the recombinant bacteriophage selectively kills the enterotoxigenic Bacteroides as compared to the commensal Bacteroides.

Further provided are methods of treating a disease or condition in a subject related to enterotoxigenic Bacteroides, the method comprising administering to the subject the recombinant bacteriophage. In some cases, the disease or condition is cancer, or any other condition associated with enterotoxigenic Bacteroides, e.g., as noted in Table 1 and/or 2.

Targeting Vancomycin-Resistant Enterococcus spp

In some embodiments, provided herein are recombinant bacteriophage comprising an exogeneous antimicrobial agent specific for a first Enterococcus, e.g., as compared to a second Enterococcus, e.g., a commensal Enterococcus. Non-limiting example first Enterococcus strains are provided in Table 2. Non-limiting example second Enterococcus are provided in Table 2.

Non-limiting example recombinant bacteriophage specific for first Enterococcus are described herein, e.g., bacteriophage including an antimicrobial agent comprising a CRISPR system. In some cases, the CRISPR system comprises a CRISPR array comprising a spacer sequence at least 80% identical to any one of SEQ ID NOS: 46-48. In some cases, the CRISPR system comprises a CRISPR array comprising a spacer sequence at least 80% identical to at least 10 contiguous nucleobases of a targetDNA of Table 2. In some embodiments, the exogeneous antimicrobial agent is specific for the first Enterococcus by targeting a target DNA sequence that is present in the first Enterococcus and not present in the second Enterococcus. In some cases, the CRISPR system comprises a nucleic acid encoding a CRISPR nuclease. As an example, the CRISPR nuclease comprises Cas3, Cas3′ and Cas3″, Cpf1, or Cas9. In some cases, the CRISPR system comprises a Type I CRISPR-Cas system, a Type II CRISPR-Cas system, or a Type V CRISPR-Cas system. As an example, the CRISPR system comprises the Type I CRISPR-Cas system. Non-limiting example Type I CRISPR-Cas systems include a Type I-A CRISPR-Cas system, a Type I-B CRISPR-Cas system, a Type I-C CRISPR-Cas system, a Type I-D CRISPR-Cas system, a Type I-E CRISPR-Cas system, and a Type I-F CRISPR-Cas system. In some cases, the Type I CRISPR-Cas system is an E. coli Type I-F system (e.g., ECIF). In some cases, the Type I CRISPR-Cas system is an E. coli Type I-E system (e.g., ECIE). In some cases, the Type I CRISPR-Cas system is a P. aeruginosa Type 1-C system (e.g., PAIC). In some cases, the CRISPR nuclease comprises a Type V nuclease, e.g., a CRISPR-Cpf1 system.

In some embodiments, the recombinant bacteriophage is prepared by a method comprising introducing into a first bacteriophage the exogeneous antimicrobial agent.

Further provided are methods of killing a first Enterococcus with the recombinant bacteriophage. Such methods include methods of selectively killing a first Enterococcus as compared to a second Enterococcus in a population of bacteria comprising the first Enterococcus and the second Enterococcus. For example, the method comprises combining with the population of bacteria the recombinant bacteriophage, where the recombinant bacteriophage selectively kills the first Enterococcus as compared to the second Enterococcus.

Further provided are methods of treating or preventing a disease or condition in a subject related to first Enterococcus, the method comprising administering to the subject the recombinant bacteriophage. In some cases, the disease or condition is the treatment of a vancomycin-resistant Enterococcus infection or prophylaxis of a potential vancomycin-resistant Enterococcus, or any other condition associated with first Enterococcus, e.g., as noted in Table 1 and/or 2.

### Bacterial Infections and Conditions

Disclosed herein, in certain embodiments, are methods of treating a condition caused by a first microbial population in a subject in need thereof. In some embodiments, the bacteriophages disclosed herein treat or prevent diseases or conditions mediated or caused by bacteria as disclosed herein in a human or animal subjects. In some embodiments, such bacteria are in contact with tissue of the subject including gut, oral cavity, lung, armpit, ocular, vaginal, anal, ear, nose, or throat tissue. In some embodiments, a bacterial infection is treated by modulating the activity of the bacteria and/or by directly killing of the first microbial population or target bacteria. In some embodiments, killing the first microbial population or target bacteria allows a second microbial population to thrive. In some embodiments, the subject is administered a favorable strain of bacteria, cocktail of bacteria, engineered bacteria, or cocktail of engineered bacteria to colonize the niche vacated by the targeted removal of the first microbial population. In some embodiments, the claim compositions increase killing of a first microbial population as compared to a second microbial population. In some embodiments, the ratio at which the antimicrobial agent kill the first microbial population with respect to the second microbial population is at least about 2:1, 3:1, 4:1, 5:1, 6:1, 7:1, 8:1, 10:1 15:1, 20:1, 30:1, 40:1, 80:1, 100:1, 1000:1, 10,000:1, or more than 100,000:1. In some embodiments, the ratio at which the antimicrobial agent kill the first microbial population with respect to the second microbial population is at most about 2:1, 3:1, 4:1, 5:1, 6:1, 7:1, 8:1, 10:1 15:1, 20:1, 30:1, 40:1, 80:1, 100:1, 1000:1, 10,000:1, or 100,000:1.

In some embodiments, one or more target bacteria present in a bacterial population are pathogenic. In some embodiments, the target bacterium is E. coli. In some embodiments, the E. coli is a multidrug-resistant (MDR) strain. An MDR strain is a bacteria strain that is resistant to at least one antibiotic. In some embodiments, a bacteria strain is resistant to an antibiotic class such as a cephalosporin, a fluoroquinolone, a carbapenem, a colistin, an aminoglycoside, vancomycin, streptomycin, and methicillin. In some embodiments, a bacteria strain is resistant to an antibiotic such as a Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, or any combination thereof. In some embodiments, the E. coli is an extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the pathogenic bacteria are uropathogenic. In some embodiments, the pathogenic bacterium is uropathogenic E. coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are enteropathogenic. In some embodiments, the pathogenic bacterium is enteropathogenic E. coli (EPEC).

In some embodiments, the bacteriophages disclosed herein are used to treat an infection, a disease, or a condition, in the gastrointestinal tract, skin, intraperitoneal cavity, colon, pancreas, bile duct, liver, or gall bladder of a subject. In some embodiments, the bacteriophages disclosed herein are used to treat a urinary tract infection (UTI). In some embodiments, the bacteriophages disclosed herein are used to treat an inflammatory disease. In some embodiments, the bacteriophages disclosed herein are used to treat an inflammatory bowel disease (IBD). In some embodiments, the bacteriophages disclosed herein are used to treat Crohn's disease. In some embodiments, the bacteriophages disclosed herein are used to treat Ulcerative colitis. In some embodiments, the infection, disease or condition is selected from the group consisting of Inflammatory bowel disease, Crohn's Disease, Ulcerative Colitis, gastrointestinal infection, gastroenteritis, dysentery, kidney failure caused by hemolytic uremic syndrome, intra-abdominal infection, food sensitization, allergies, autism, and acne.

In some embodiments, the bacteriophages disclosed herein are used to treat an infection, a disease, or a condition, in the urinary tract of a subject. In some embodiments, the bacteriophages are used to modulate and/or kill target bacteria within the urinary tract flora of a subject. In some embodiments, the bacteriophages are used to selectively modulate and/or kill one or more target uropathogenic bacteria from a plurality of bacteria within the urinary tract flora of a subject. In some embodiments, the target bacterium is uropathogenic E. coli (UPEC). In some embodiments, the subject is administered a favorable strain of bacteria, cocktail of bacteria, engineered bacteria, or cocktail of engineered bacteria to colonize the niche vacated by the targeted removal of the first microbial population.

In some embodiments, the bacteriophages disclosed herein are used to treat Inflammatory Bowel Disease. In some embodiments, the Inflammatory Bowel Disease is Crohn's Disease or ulcerative colitis. In some embodiment, an additional therapeutic is used to treat the subject. In some embodiments, the additional therapeutic comprises oral corticosteroids; oral aminosalicylates [e.g., mesalamine (Asacol HD, Delzicol, others), balsalazide (Colazal), and olsalazine (Dipentum)]; TNF inhibitos [e.g., Remicade (infliximab), Enbrel (etanercept), Humira (adalimumab), Cimzia (certolizumab pegol) and Simponi (golimumab)]; immunosuppressants [e.g., azathioprine (Azasan, Imuran), mercaptopurine (Purinethol, Purixan), cyclosporine (Gengraf, Neoral, Sandimmune), and methotrexate (Trexall)]; and integrin/integrin receptor antagonists [e.g., natalizumab (Tysabri), vedolizumab (Entyvio), ustekinumab (Stelara), and guselkumab (Tremfya)]. In some embodiments,

In some embodiments, the subject may have colorectal cancer. In some embodiments, an additional therapeutic is used to treat the subject. In some embodiments, the additional therapeutic comprises 5-Fluorouracil, leucovorin, oxaliplatin, bevacizumab, panitumumab, cetuximab, capecitabine, irinotecan, ziv-aflibercept, ramucirumab, regorafenib, trifluridine, tipiracil, pembrolizumab, nivolumab, ipilimumab, trastuzumab, pertuzumab, lapatinib, encorafenib, Larotrectinib, or entrectinib. In some embodiments, the subject is administered a favorable strain of bacteria, cocktail of bacteria, engineered bacteria, or cocktail of engineered bacteria to colonize the niche vacated by the targeted removal of the first microbial population.

In some embodiments, the subject may have a gastrointestinal infection, gastroenteritis, dysentery, or kidney failure caused by hemolytic uremic syndrome. In some embodiments, the subject may have an intra-abdominal infection. In some embodiments, the intra-abdominal infection comprises peritonitis, diverticulitis, cholecystitis, cholangitis, pancreatitis, or any combination thereof. In some embodiments, an additional therapeutic is used to treat the subject. In some embodiments, the additional therapeutic comprises an antibiotic. In some embodiments, an antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides or tetracycline. In some embodiments, an antibiotic agent described herein is an aminoglycoside such as Amikacin, Gentamicin, Kanamycin, Neomycin, Netilmicin, Tobramycin, or Paromomycin. In some embodiments, an antibiotic agent described herein is an Ansamycin such as Geldanamycin or Herbimycin. In some embodiments, an antibiotic agent described herein is a carbacephem such as Loracarbef. In some embodiments, an antibiotic agent described herein is a carbapenem such as Ertapenem, Doripenem, Imipenem/Cilastatin, or Meropenem. In some embodiments, an antibiotic agent described herein is a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil, or Cefuroxime. In some embodiments, an antibiotic agent is a Cephalosporins (third generation) such as Cefixime, Cefdinir, Cefditoren, Cefoperazone, Cefotaxime, Cefpodoxime, Ceftibuten, Ceftizoxime, and Ceftriaxone, or a Cephalosporins (fourth generation) such as Cefepime or Ceftobiprole. In some embodiments, an antibiotic agent described herein is a lincosamide, such as Clindamycin, and Azithromycin, or a macrolide, such as Azithromycin, Clarithromycin, Dirithromycin, Erythromycin, Roxithromycin, Troleandomycin, Telithromycin, and Spectinomycin. In some embodiments, an antibiotic agent described herein is a monobactams such as Aztreonam, or a nitrofuran such as Furazolidone or Nitrofurantoin. In some embodiments, an antibiotic agent described herein is a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin, and Ticarcillin. In some embodiments, an antibiotic agent described herein is a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, or Trimethoprim-Sulfamethoxazole (Co-trimoxazole) (TMP-SMX). In some embodiments, an antibiotic agent described herein is a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. In some embodiments, an antibiotic agent described herein is a polypeptide such as Bacitracin, Colistin, or Polymyxin B. In some embodiments, an antibiotic agent described herein is a tetracycline such as Demeclocycline, Doxycycline, Minocycline, or Oxytetracycline. In some embodiments, the subject is administered a favorable strain of bacteria, cocktail of bacteria, engineered bacteria, or cocktail of engineered bacteria to colonize the niche vacated by the targeted removal of the first microbial population.

In some embodiments, the subject may have gastrointestinal infection, gastroenteritis, or Parkinson's disease. In some embodiments, the subject is administered an additional therapeutic. In some embodiments, the additional therapeutic is an antibiotic. In some embodiments, the additional therapeutic is carbidopa-levodopa. In some embodiments, the subject is administered a favorable strain of bacteria, cocktail of bacteria, engineered bacteria, or cocktail of engineered bacteria to colonize the niche vacated by the targeted removal of the first microbial population.

In some embodiments, the subject may have a cancer or a tumor. In some embodiments, the cancer or tumor comprises melanoma, non-small cell lung cancer, renal cell carcinoma, urothelial carcinoma, colorectal cancer, hepatocellular cancer, stomach cancer, large B cell lymphoma, cervical cancer, small cell lung cancer, esophageal cancer, endometrial carcinoma, cutaneous squamous cell carcinoma, breast cancer, Hodgkin lymphoma, non-Hodgkin lymphoma, head & neck cancer, Merkel cell carcinoma, microsatellite instability (MSI)-high/deficient mismatch repair (dMMR) tumors, tumor mutational burden (TMB)-high tumors, and any other tumor types approved to be treated with immunotherapies (e.g., immune checkpoint inhibitors). In some embodiments, the subject is administered an additional therapeutic. In some embodiments, the subject is administered an immune checkpoint inhibitor. In some embodiments, the immune checkpoint inhibitor comprises pembrolizumab, nivolumab, ipilumumab, atezolizumab, avelumab, durvalumab, or cemiplimab-rwlc. In some embodiments, the subject is administered a favorable strain of bacteria, cocktail of bacteria, engineered bacteria, or cocktail of engineered bacteria to colonize the niche vacated by the targeted removal of the first microbial population.

In some embodiments, the subject may have acnevulgaris. In some embodiments, the subject is administered an additional therapeutic. In some embodiments, the additional therapeutic comprises antibiotics, salicylic acid, azelaic acid, dapsone (Aczone) gel, retinoids, isotretinoin anti-androgen agents, laser therapy, chemical peels, acne extraction techniques, steroids, or a combination thereof. In some embodiments, the subject is administered a favorable strain of bacteria, cocktail of bacteria, engineered bacteria, or cocktail of engineered bacteria to colonize the niche vacated by the targeted removal of the first microbial population.

In some embodiments, the subject may develop food allergies or autism. In some embodiments, the subject is administered an additional therapeutic. In some embodiments, the additional therapeutic comprises an antibiotic. In some embodiments, the subject is administered

In some embodiments, the subject may have anxiety, depression, or cognitive dysfunction. In some embodiments, the subject is administered an additional therapeutic. In some embodiments, the additional therapeutic comprises an antibiotic or an antidepressant. In some embodiments, the subject is administered a favorable strain of bacteria, cocktail of bacteria, engineered bacteria, or cocktail of engineered bacteria to colonize the niche vacated by the targeted removal of the first microbial population. In some embodiments, the second microbial population engineered bacteria t colonize the niche vacated by the targeted removal of the first microbial population.

In some embodiments, non-limiting examples of target bacteria includes Escherichia spp., Salmonella spp., Bacillus spp., Corynebacterium Clostridium spp., Clostridium spp., Pseudomonas spp., Clostridium spp., Lactococcus spp., Acinetobacter spp., Mycobacterium spp., Myxococcus spp., Staphylococcus spp., Streptococcus spp., or cyanobacteria. In some embodiments, non-limiting examples of bacteria include Escherichia coli, Salmonella enterica, Bacillus subtilis, Clostridium acetobutylicum, Clostridium ljungdahlii, Clostridium difficile, Acinetobacter baumannii, Mycobacterium tuberculosis, Myxococcus xanthus, Staphylococcus aureus, Streptococcus pyogenes, or cyanobacteria. In some embodiments, non-limiting examples of bacteria include Staphylococcus aureus, methicillin resistant Staphylococcus aureus, Streptococcus pneumonia, carbapenem-resistant Enterobacteriaceae, Staphylococcus epidermidis, Staphylococcus salivarius, Corynebacterium minutissium, Corynebacterium pseudodiphtheriae, Corynebacterium stratium, Corynebacterium group G1, Corynebacterium group G2, Streptococcus pneumonia, Streptococcus mitis, Streptococcus sanguis, Escherichia coli, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces spp., Porphyromonas spp., Prevotella melaninogenicus, Helicobacter pylori, Helicobacter felis, or Campylobacter jejuni. Further non-limiting examples of bacteria include lactic acid bacteria including, but not limited to, Lactobacillus spp. and Bifidobacterium spp.; electrofuel bacterial strains including but not limited to Geobacter spp., Clostridium spp., or Ralstonia eutropha; or bacteria pathogenic on, for example, plants and mammals. In some embodiments, the bacterium is Escherichia coli. In some embodiments, the bacterium is Clostridium difficile.

In some embodiments, the pathogenic bacteria are various strains of C. difficile including: CD043, CD05, CD073, CD093, CD180, CD106, CD128, CD199, CD111, CD108, CD25, CD148, CD154, FOBT195, CD03, CD038, CD112, CD196, CD105, UK1, UK6, BI-9, CD041, CD042, CD046, CD19, or R20291.

In some embodiments, the one or more target bacteria present in the bacterial population form a biofilm. In some embodiments, the biofilm comprises pathogenic bacteria. In some embodiments, the bacteriophage disclosed herein is used to treat a biofilm.

In some embodiments, the methods and compositions disclosed herein are for use in veterinary and medical applications as well as research applications.

### Microbiome

“Microbiome”, “microbiota”, and “microbial habitat” are used interchangeably hereinafter and refer to the ecological community of microorganisms that live on or in a subject's bodily surfaces, cavities, and fluids. Non-limiting examples of habitats of microbiome include: gut, colon, skin, skin surfaces, skin pores, vaginal cavity, umbilical regions, conjunctival regions, intestinal regions, stomach, nasal cavities and passages, gastrointestinal tract, urogenital tracts, saliva, mucus, and feces. In some embodiments, the microbiome comprises microbial material including, but not limited to, bacteria, archaea, protists, fungi, and viruses. In some embodiments, the microbial material comprises a gram-negative bacterium. In some embodiments, the microbial material comprises a gram-positive bacterium. In some embodiments, the microbial material comprises Proteobacteria, Actinobacteria, Bacteriodetes, or Firmicutes.

In some embodiments, the bacteriophages as disclosed herein are used to modulate or kill a first microbial population within the microbiome of a subject. In some embodiments, the bacteriophages are used to modulate and/or kill a first microbial population within the microbiome by the CRISPR-Cas system, lytic activity, a peptide encoded by a sequenced comprised in the bacteriophage or a combination thereof. In some embodiments, the bacteriophages are used to modulate and/or kill a first microbial population within the microbiome of a subject. In some embodiments, the bacteriophages are used to selectively modulate and/or kill one or more microbial species from a plurality of bacteria within the microbiome of a subject. In some embodiments, modulating and/or killing the first microbial population allows a second microbial population to thrive. In some embodiments, the first microbial bacterium is E. coli. In some embodiments, the E. coli comprises an adherent-invasive E. coli (AIEC) strain. In some embodiments, the E. coli comprises apks+genomic sequence. In some embodiments, the E. coli comprises a shiga toxin-producing E. coli (STEC) (e.g., E. coli 0157), verocytotoxin-producing E. coli (VTEC), or enterohemorrhagic E. coli (EHEC) strains. In some embodiments, the E. coli is a multidrug-resistant (MDR) strain. In some embodiments, the E. coli is an extended spectrum beta-lactamase (ESBL) strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the pathogenic bacteria are uropathogenic. In some embodiments, the pathogenic bacterium is uropathogenic E. coli (UPEC). In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are entereopathogenic. In some embodiments, the pathogenic bacterium is entereopathogenic E. coli (EPEC). In some embodiments, the first microbial population comprises Bacteroides. In some, embodiments, the Bacteroides species comprises B. fragilis or B. thetaiotaomicron. In some embodiments, the B. fragilis comprises an enterotoxigenic B. fragilis. In some embodiments, the first microbial population comprises a Enterococcus. In some embodiments, the Enterococcus species comprises E. faecalis, or E. faecium. In some embodiments, the first microbial population compresses Shigella. In some embodiments, the first microbial population comprises Hafnia alvei. In some embodiments, the first microbial population comprises Pseduomonas aerugonosa. In some embodiments, the first microbial population comprises Mongsnella morganii. In some embodiments, the first microbial population comprises Pseudomonas putida. In some embodiments, the first microbial population comprises Citrobacter koseri. In some embodiments, the first microbial population comprises Klebsiella pneumonia. In some embodiments, the first microbial population comprises Campylobacter jejuni. In some embodiments, the first microbial population comprises Mycobacterium avium subspecies paratuberculosis.

In some embodiments, the second microbial population is selected from the group consisting of E. coli, Bacteroides, Enterococcus, Shigella, Hafnia alvei, Pseudomonas aeruginosa, Morganella morganii, Pseudomonas putida, Citrobacter koseri, and Klebsiella pneumonia, and Campylobacter jejuni. In some embodiments, the first microbial population comprises E. coli. In some embodiments, the E. coli is a commensal E. coli. In some embodiments, the first microbial population comprises Bacteroides. In some embodiments, the B. fragilis comprises a commensal B. fragilis. In some embodiments, the first microbial population comprises an Enterococcus. In some embodiments, the Enterococcus species is commensal. In some embodiments, the first microbial population compresses Shigella. In some embodiments, the first microbial population comprises Hafnia alvei. In some embodiments, the first microbial population comprises Pseduomonas aerugonosa In some embodiments, the first microbial population comprises Mongsnella morganii. In some embodiments, the first microbial population comprises Pseudomonas putida. In some embodiments, the first microbial population comprises Citrobacter koseri. In some embodiments, the first microbial population comprises Klebsiella pneumonia. In some embodiments, the first microbial population comprises Campylobacter jejuni. In some embodiments, the first microbial population comprises Mycobacterium avium subspecies paratuberculosis.

In some embodiments, the bacteriophages are used to modulate or kill target single or plurality of bacteria within the microbiome or gut flora of the gastrointestinal tract, skin, intraperitoneal cavity, colon, pancreas, bile duct, liver, and gall bladder. of a subject. Modification (e.g., dysbiosis) of the microbiome or gut flora increases the risk for health conditions such as diabetes, mental disorders, ulcerative colitis, cancer, autoimmune disorders, obesity, diabetes, diseases of the central nervous system and inflammatory bowel disease, Crohn's Disease, gastrointestinal infection, gastroenteritis, dysentery, kidney failure caused by hemolytic uremic syndrome, intra-abdominal infection, food sensitization, allergies, autism, and acne. A bacteria associated with diseases and conditions in a subject and are being modulated or killed by the bacteriophages include strains, sub-strains, and enterotypes of E. coli, Bacteroides, Enterococcus, Shigella, Hafnia alvei, Pseudomonas aeruginosa, Morganella morganii, Pseudomonas putida, Citrobacter koseri, and Klebsiella pneumonia, Campylobacter jejuni, and Mycobacterium avium subspecies paratuberculosis.

In some embodiments, a bacteriophage disclosed herein is administered to a subject to promote a healthy microbiome. In some embodiments, a bacteriophage disclosed herein is administered to a subject to restore a subject's microbiome to a microbiome composition that promotes health. In some embodiments, a composition comprising a bacteriophage disclosed herein comprises a prebiotic, a probiotic, or a third agent. In some embodiment, microbiome related disease or disorder is treated by a bacteriophage disclosed herein.

### Administration Routes and Dosage

Dose and duration of the administration of a composition disclosed herein will depend on a variety of factors, including the subject's age, subject's weight, and tolerance of the phage. In some embodiments, a bacteriophage disclosed herein is administered to patients through a route selected from the group consisting of intramuscular, intravenous, sub cutaneous, oral, transmucosal, buccal, sublingual, rectal, intranasal, and topical.

In some embodiments, a bacteriophage disclosed herein is administered to patients by oral administration.

In some embodiments, a dose of phage between 10³ and 10²⁰ PFU is given. In some embodiments, a dose of phage between 10³ and 10¹⁰ PFU is given. In some embodiments, a dose of phage between 10⁶ and 10²⁰ PFU is given. In some embodiments, a dose of phage between 10⁶ and 10¹⁰ PFU is given. For example, in some embodiments, the bacteriophage is present in a composition in an amount between 10³ and 10¹¹ PFU. In some embodiments, the bacteriophage is present in a composition in an amount about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴ PFU, or more. In some embodiments, the bacteriophage is present in a composition in an amount of less than 10 PFU. In some embodiments, the bacteriophage is present in a composition in an amount between 10¹ and 10⁸, 10⁴ and 10⁹, 10⁵ and 10¹⁰, or 10⁷ and 10¹¹ PFU.

In some embodiments, a bacteriophage or a mixture is administered to a subject in need thereof 1, 2, 3, 4, 5, 6, 7, 8, 9, 10, 11, 12, 13, 14, 15, 16, 17, 18, 19, 20, 21, 22, 23, or 24 times a day. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, or 21 times a week. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month. In some embodiments, a bacteriophage or a mixture is administered to a subject in need thereof every 2, 4, 6, 8, 10, 12, 14, 18, 20, 22, or 24 hours.

In some embodiments, the compositions (for example bacteriophage compositions) disclosed herein are administered before, during, or after the occurrence of a disease or condition. In some embodiment, the timing of administering the composition containing the bacteriophage varies. In some embodiments, the pharmaceutical compositions are used as a prophylactic and are administered continuously to subjects with a propensity to conditions or diseases in order to prevent the occurrence of the disease or condition. In some embodiments, pharmaceutical compositions are administered to a subject during or as soon as possible after the onset of the symptoms. In some embodiments, the administration of the compositions is initiated within the first 48 hours of the onset of the symptoms, within the first 24 hours of the onset of the symptoms, within the first 6 hours of the onset of the symptoms, or within 3 hours of the onset of the symptoms. In some embodiments, the initial administration of the composition is via any route practical, such as by any route described herein using any formulation described herein. In some embodiments, the compositions is administered as soon as is practicable after the onset of a disease or condition is detected or suspected, and for a length of time necessary for the treatment of the disease, such as, for example, from about 1 month to about 3 months. In some embodiments, the length of treatment will vary for each subject.

### Environmental Therapy

In some embodiments, bacteriophages disclosed herein are further used for food and agriculture sanitation (including meats, fruits and vegetable sanitation), hospital sanitation, home sanitation, vehicle and equipment sanitation, industrial sanitation, etc. In some embodiments, bacteriophages disclosed herein are used for the removal of antibiotic-resistant or other undesirable pathogens from medical, veterinary, animal husbandry, or any additional environments bacteria are passed to humans or animals.

Environmental applications of phage in health care institutions are for equipment such as endoscopes and environments such as ICUs which are potential sources of nosocomial infection due to pathogens that are difficult or impossible to disinfect. In some embodiments, a phage disclosed herein is used to treat equipment or environments inhabited by bacterial genera which become resistant to commonly used disinfectants. In some embodiments, phage compositions disclosed herein are used to disinfect inanimate objects. In some embodiments, an environment disclosed herein is sprayed, painted, or poured onto with aqueous solutions with phage titers. In some embodiment a solution described herein comprises between 10¹-10²⁰ plaque forming units (PFU)/ml. In some embodiments, a bacteriophage disclosed herein is applied by aerosolizing agents that include dry dispersants to facilitate distribution of the bacteriophage into the environment. In some embodiments, objects are immersed in a solution containing bacteriophage disclosed herein.

### Sanitation

In some embodiments, bacteriophages disclosed herein are used as sanitation agents in a variety of fields. Although the terms “phage” or “bacteriophage” may be used, it should be noted that, where appropriate, this term should be broadly construed to include a single bacteriophage, multiple bacteriophages, such as a bacteriophage mixtures and mixtures of a bacteriophage with an agent, such as a disinfectant, a detergent, a surfactant, water, etc.

In some embodiments, bacteriophages are used to sanitize hospital facilities, including operating rooms, patient rooms, waiting rooms, lab rooms, or other miscellaneous hospital equipment. In some embodiments, this equipment includes electrocardiographs, respirators, cardiovascular assist devices, intraaortic balloon pumps, infusion devices, other patient care devices, televisions, monitors, remote controls, telephones, beds, etc. In some situations, the bacteriophage is applied through an aerosol canister. In some embodiments, bacteriophage is applied by wiping the phage on the object with a transfer vehicle.

In some embodiments, a bacteriophage described herein is used in conjunction with patient care devices. In some embodiment, bacteriophage is used in conjunction with a conventional ventilator or respiratory therapy device to clean the internal and external surfaces between patients. Examples of ventilators include devices to support ventilation during surgery, devices to support ventilation of incapacitated patients, and similar equipment. In some embodiments, the conventional therapy includes automatic or motorized devices, or manual bag-type devices such as are commonly found in emergency rooms and ambulances. In some embodiments, respiratory therapy includes inhalers to introduce medications such as bronchodilators as commonly used with chronic obstructive pulmonary disease or asthma, or devices to maintain airway patency such as continuous positive airway pressure devices.

In some embodiment, a bacteriophage described herein is used to cleanse surfaces and treat colonized people in an area where highly-contagious bacterial diseases, such as meningitis or enteric infections are present.

In some embodiments, water supplies are treated with a composition disclosed herein. In some embodiments, bacteriophage disclosed herein is used to treat contaminated water, water found in cisterns, wells, reservoirs, holding tanks, aqueducts, conduits, and similar water distribution devices. In some embodiments, the bacteriophage is applied to industrial holding tanks where water, oil, cooling fluids, and other liquids accumulate in collection pools. In some embodiments, a bacteriophage disclosed herein is periodically introduced to the industrial holding tanks in order to reduce bacterial growth.

In some embodiments, bacteriophages disclosed herein are used to sanitize a living area, such as a house, apartment, condominium, dormitory, or any living area. In some embodiments, the bacteriophage is used to sanitize public areas, such as theaters, concert halls, museums, train stations, airports, pet areas, such as pet beds, or litter boxes. In this capacity, the bacteriophage is dispensed from conventional devices, including pump sprayers, aerosol containers, squirt bottles, pre-moistened towelettes, etc, applied directly to (e.g., sprayed onto) the area to be sanitized, or be transferred to the area via a transfer vehicle, such as a towel, sponge, etc. In some embodiments, a phage disclosed herein is applied to various rooms of a house, including the kitchen, bedrooms, bathrooms, garage, basement, etc. In some embodiments, a phage disclosed herein is in the same manner as conventional cleaners. In some embodiments, the phage is applied in conjunction with (before, after, or simultaneously with) conventional cleaners provided that the conventional cleaner is formulated so as to preserve adequate bacteriophage biologic activity.

In some embodiments, a bacteriophage disclosed herein is added to a component of paper products, either during processing or after completion of processing of the paper products. Paper products to which a bacteriophage disclosed herein is added include, but are not limited to, paper towels, toilet paper, moist paper wipes.

### Food Safety

In some embodiments, a bacteriophage described herein is used in any food product or nutritional supplement, for preventing contamination. Examples for food or pharmaceuticals products are milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

The broad concept of bacteriophage sanitation is applicable to other agricultural applications and organisms. Produce, including fruits and vegetables, dairy products, and other agricultural products. For example, freshly-cut produce frequently arrive at the processing plant contaminated with pathogenic bacteria. This has led to outbreaks of food-borne illness traceable to produce. In some embodiments, the application of bacteriophage preparations to agricultural produce substantially reduce or eliminate the possibility of food-borne illness through application of a single phage or phage mixture with specificity toward specie s of bacteria associated with food-borne illness. In some embodiments, bacteriophages are applied at various stages of production and processing to reduce bacterial contamination at that point or to protect against contamination at subsequent points.

In some embodiments, specific bacteriophages are applied to produce in restaurants, grocery stores, produce distribution centers. In some embodiments, bacteriophages disclosed herein are periodically or continuously applied to the fruit and vegetable contents of a salad bar. In some embodiments, the application of bacteriophages to a salad bar or to sanitize the exterior of a food item is a misting or spraying process or a washing process.

In some embodiments, a bacteriophage described herein is used in matrices or support media containing with packaging containing meat, produce, cut fruits and vegetables, and other foodstuffs. In some embodiments, polymers that are suitable for packaging are impregnated with a bacteriophage preparation.

In some embodiments, a bacteriophage described herein is used in farm houses and livestock feed. In some embodiments, on a farm raising livestock, the livestock is provided with bacteriophage in their drinking water, food, or both. In some embodiments, a bacteriophage described herein is sprayed onto the carcasses and used to disinfect the slaughter area.

The use of specific bacteriophages as biocontrol agents on produce provides many advantages. For example, bacteriophages are natural, non-toxic products that will not disturb the ecological balance of the natural microflora in the way the common chemical sanitizers do, but will specifically lyse the targeted food-borne pathogens. Because bacteriophages, unlike chemical sanitizers, are natural products that evolve along with their host bacteria, new phages that are active against recently emerged, resistant bacteria are rapidly identified when required, whereas identification of a new effective sanitizer is a much longer process, several years.

### Pharmaceutical Compositions

Disclosed herein, in certain embodiments, are pharmaceutical compositions comprising (a) the nucleic acid sequences as disclosed herein; and (b) a pharmaceutically acceptable excipient. Also disclosed herein, in certain embodiments, are pharmaceutical compositions comprising (a) the bacteriophages as disclosed herein; and (b) a pharmaceutically acceptable excipient. Further disclosed herein, in certain embodiments, are pharmaceutical compositions comprising (a) the compositions as disclosed herein; and (b) a pharmaceutically acceptable excipient.

In some embodiments, the disclosure provides pharmaceutical compositions and methods of administering the same to treat bacterial, archaeal infections or to disinfect an area. In some embodiments, the pharmaceutical composition comprises any of the reagents discussed above in a pharmaceutically acceptable carrier. In some embodiments, a pharmaceutical composition or method disclosed herein treats urinary tract infections (UTI) and/or inflammatory diseases (e.g. inflammatory bowel disease (IBD)). In some embodiments, a pharmaceutical composition or method disclosed herein treats Crohn's disease. In some embodiments, a pharmaceutical composition or method disclosed herein treats Ulcerative colitis. In some embodiments, a pharmaceutical composition or method disclosed herein treats gastrointestinal infection, gastroenteritis, dysentery, kidney failure caused by hemolytic uremic syndrome, intra-abdominal infection, food sensitization, allergies, autism, or acne. In some embodiments, a pharmaceutical composition or method disclosed herein treats a disease or condition disclosed herein.

In some embodiments, compositions disclosed herein comprise medicinal agents, pharmaceutical agents, carriers, adjuvants, dispersing agents, diluents, and the like.

In some embodiments, the bacteriophages disclosed herein are formulated for administration in a pharmaceutical carrier in accordance with suitable methods. In some embodiments, the manufacture of a pharmaceutical composition according to the disclosure, the bacteriophage is admixed with, inter alia, an acceptable carrier. In some embodiments, the carrier is a solid (including a powder) or a liquid, or both, and is preferably formulated as a unit-dose composition. In some embodiments, one or more bacteriophages are incorporated in the compositions disclosed herein, which are prepared by any suitable method of a pharmacy.

In some embodiment, a method of treating subject's in-vivo, comprising administering to a subject a pharmaceutical composition comprising a bacteriophage disclosed herein in a pharmaceutically acceptable carrier, wherein the pharmaceutical composition is administered in a therapeutically effective amount. In some embodiments, the administration of the bacteriophage to a human subject or an animal in need thereof are by any means known in the art.

In some embodiments, bacteriophages disclosed herein are for oral administration. In some embodiments, the bacteriophages are administered in solid dosage forms, such as capsules, tablets, and powders, or in liquid dosage forms, such as elixirs, syrups, and suspensions. In some embodiments, compositions and methods suitable for buccal (sub-lingual) administration include lozenges comprising the bacteriophages in a flavored base, usually sucrose and acacia or tragacanth; and pastilles comprising the bacteriophages in an inert base such as gelatin and glycerin or sucrose and acacia.

In some embodiments, methods and compositions of the present disclosure are suitable for parenteral administration comprising sterile aqueous and non-aqueous injection solutions of the bacteriophage. In some embodiments, these preparations are isotonic with the blood of the intended recipient. In some embodiments, these preparations comprise antioxidants, buffers, bacteriostals and solutes which render the composition isotonic with the blood of the intended recipient. In some embodiments, aqueous and non-aqueous sterile suspensions include suspending agents and thickening agents. In some embodiments, compositions disclosed herein are presented in unit\dose or multi-dose containers, for example sealed ampoules and vials, and are stored in a freeze-dried (lyophilized) condition requiring only the addition of the sterile liquid carrier, for example, saline or water for injection on immediately prior to use.

In some embodiment, methods and compositions suitable for rectal administration are presented as unit dose suppositories. In some embodiments, these are prepared by admixing the bacteriophage with one or more conventional solid carriers, for example, cocoa butter, and then shaping the resulting mixture. In some embodiments, methods and compositions suitable for topical application to the skin are in the form of an ointment, cream, lotion, paste, gel, spray, aerosol, or oil. In some embodiments, carriers which are used include petroleum jelly, lanoline, polyethylene glycols, alcohols, transdermal enhancers, and combinations of two or more thereof.

In some embodiments, methods and compositions suitable for transdermal administration are presented as discrete patches adapted to remain in intimate contact with the epidermis of the recipient for a prolonged period of time.

In some embodiments, methods and compositions suitable for nasal administration or otherwise administered to the lungs of a subject include any suitable means, e.g., administered by an aerosol suspension of respirable particles comprising the bacteriophage compositions, which the subject inhales. In some embodiments, the respirable particles are liquid or solid. As used herein, “aerosol” includes any gas-borne suspended phase, which is capable of being inhaled into the bronchioles or nasal passages. In some embodiments, aerosols of liquid particles are produced by any suitable means, such as with a pressure-driven aerosol nebulizer, or an ultrasonic nebulizer. In some embodiments, aerosols of solid particles comprising the composition is produced with any solid particulate medicament aerosol generator, by techniques known in the pharmaceutical art.

In some embodiment, methods and compositions suitable for administering bacteriophages disclosed herein to a surface of an object or subject includes aqueous solutions. In some embodiments, such aqueous solutions are sprayed onto the surface of an object or subject. In some embodiment, the aqueous solutions are used to irrigate and clean a physical wound of a subject form foreign debris including bacteria.

In some embodiments, the bacteriophages disclosed herein are administered to the subject in a therapeutically effective amount. In some embodiments, at least one bacteriophage composition disclosed herein is formulated as a pharmaceutical formulation. In some embodiments, a pharmaceutical formulation comprises 1, 2, 3, 4, 5, 6, 7, 8, 9, 10, 11, 12, 13, 14, 15, 16, 17, 18, 19, 20, or more bacteriophage disclosed herein. In some instances, a pharmaceutical formulation comprises a bacteriophage described herein and at least one of: an excipient, a diluent, or a carrier.

In some embodiments, a pharmaceutical formulation comprises an excipient. Excipients are described in the Handbook of Pharmaceutical Excipients, American Pharmaceutical Association (1986) and includes but are not limited to solvents, dispersion media, diluents, or other liquid vehicles, dispersion or suspension aids, surface active agents, isotonic agents, thickening or emulsifying agents, preservatives, solid binders, and lubricants.

Non-limiting examples of suitable excipients include but is not limited to a buffering agent, a preservative, a stabilizer, a binder, a compaction agent, a lubricant, a chelator, a dispersion enhancer, a disintegration agent, a flavoring agent, a sweetener, or a coloring agent.

In some embodiments, an excipients a buffering agent. Non-limiting examples of suitable buffering agents include but is not limited to sodium citrate, magnesium carbonate, magnesium bicarbonate, calcium carbonate, and calcium bicarbonate. In some embodiments, a pharmaceutical formulation comprises any one or more buffering agent listed: sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide, and other calcium salts.

In some embodiments an excipient is a preservative. Non-limiting examples of suitable preservatives include but is not limited to antioxidants, such as alpha-tocopherol and ascorbate, and antimicrobials, such as parabens, chlorobutanol, and phenol. In some embodiments, antioxidants include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol, and N-acetyl cysteine. In some embodiments, preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, protease inhibitor, reducing agent, alkylating agent, antimicrobial agent, oxidase inhibitor, or other inhibitor.

In some embodiments, a pharmaceutical formulation comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the binders that are used in a pharmaceutical formulation are selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water, or a combination thereof.

In some embodiments, a pharmaceutical formulation comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethylene glycol, sodiumbenzoate, sodium lauryl sulfate, magnesium lauryl sulfate, and light mineral oil. In some embodiments, lubricants that are in a pharmaceutical formulation are selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc, or a combination thereof.

In some embodiments, an excipient comprises a flavoring agent. In some embodiments, flavoring agents includes natural oils; extracts from plants, leaves, flowers, and fruits; and combinations thereof.

In some embodiments, an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some instances, a pharmaceutical formulation comprises a coloring agent. Non-limiting examples of suitable color agents include food, drug and cosmetic colors (FD&C), drug and cosmetic colors (D&C), and external drug and cosmetic colors (Ext. D&C).

In some embodiments, the pharmaceutical formulation disclosed herein comprises a chelator. In some embodiments, a chelator includes ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA.

In some instances, a pharmaceutical formulation comprises a diluent. Non-limiting examples of diluents include water, glycerol, methanol, ethanol, and other similar biocompatible diluents. In some embodiments, a diluent is an aqueous acid such as acetic acid, citric acid, maleic acid, hydrochloric acid, phosphoric acid, nitric acid, sulfuric acid, or similar.

In some embodiments, a pharmaceutical formulation comprises a surfactant. In some embodiments, surfactants are be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids, or a combination thereof.

In some instances, a pharmaceutical formulation comprises an additional pharmaceutical agent. In some embodiments, an additional pharmaceutical agent is an antibiotic agent. In some embodiments, an antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides, or tetracycline.

In some embodiments, an antibiotic agent described herein is an aminoglycoside such as Amikacin, Gentamicin, Kanamycin, Neomycin, Netilmicin, Tobramycin, or Paromomycin. In some embodiments, an antibiotic agent described herein is an Ansamycin such as Geldanamycin, or Herbimycin.

In some embodiments, an antibiotic agent described herein is a carbacephem such as Loracarbef. In some embodiments, an antibiotic agent described herein is a carbapenem such as Ertapenem, Doripenem, Imipenem/Cilastatin, or Meropenem.

In some embodiments, an antibiotic agent described herein is a cephalosporins (first generation) such as Cefadroxil, Cefazolin, Cefalexin, Cefalotin, or Cefalothin, or alternatively a Cephalosporins (second generation) such as Cefaclor, Cefamandole, Cefoxitin, Cefprozil or Cefuroxime. In some embodiments, an antibiotic agent is a Cephalosporins (third generation) such as Cefixime, Cefdinir, Cefditoren, Cefoperazone, Cefotaxime, Cefpodoxime, Ceftibuten, Ceftizoxime, and Ceftriaxone, or a Cephalosporins (fourth generation), such as Cefepime or Ceftobiprole.

In some embodiments, an antibiotic agent described herein is a lincosamide such as Clindamycin and Azithromycin, or a macrolide such as Azithromycin, Clarithromycin, Dirithromycin, Erythromycin, Roxithromycin, Troleandomycin, Telithromycin, and Spectinomycin.

In some embodiments, an antibiotic agent described herein is a monobactams such as Aztreonam, or a nitrofuran, such as Furazolidone or Nitrofurantoin.

In some embodiments, an antibiotic agent described herein is a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin, and Ticarcillin.

In some embodiments, an antibiotic agent described herein is a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, or Trimethoprim-Sulfamethoxazole (Co-trimoxazole) (TMP-SMX).

In some embodiments, an antibiotic agent described herein is a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin.

In some embodiments, an antibiotic agent described herein is a polypeptide such as Bacitracin, Colistin, or Polymyxin B.

In some embodiments, an antibiotic agent described herein is a tetracycline such as Demeclocycline, Doxycycline, Minocycline, or Oxytetracycline.

### Enumerated Embodiments

1. A recombinant bacteriophage capable of infecting a first microbial population and a second microbial population, the recombinant bacteriophage comprising a nucleic acid sequence encoding an exogenous antimicrobial agent that targets and kills the first microbial population, wherein the recombinant bacteriophage kills the first microbial population to a greater degree as compared to the second microbial population.

2. The recombinant bacteriophage of embodiment 1, wherein the recombinant bacteriophage is P1 phage, a M13 phage, a λ phage, a T4 phage, a T7 phage, a T7m phage, a φC2 phage, a φCD27 phage, a φNM1 phage, Bc431 v3 phage, 410 phage, 425 phage, 4151 phage, A511-like phages, B054, 0176-like phages, or Campylobacter phages (such as NCTC 12676 and NCTC 12677).

3. The recombinant bacteriophage of any one of embodiments 1-2, wherein the recombinant bacteriophage is a recombinant lytic bacteriophage.

4. The recombinant bacteriophage of any one of embodiments 1-2, wherein the recombinant bacteriophage comprises a second nucleic acid sequence encoding an operable lytic gene capable of inducing lysis of the first microbial population or the second microbial population during a lytic cycle of the recombinant bacteriophage.

5. The recombinant bacteriophage of any one of embodiments 2-4, wherein the recombinant bacteriophage is rendered lytic by the removal, replacement, or inactivation of at least one lysogenic gene.

6. The recombinant bacteriophage of embodiment 5, wherein the lysogenic gene is selected from the group consisting of cI repressor, cII, lexA, and int.

7. The recombinant bacteriophage of any one of embodiments 1-6, wherein the recombinant bacteriophage is replication competent.

8. The recombinant bacteriophage of any one of embodiments 1-7, wherein the exogenous antimicrobial agent comprises an endonuclease or an exonuclease or an antibiotic peptide or a biologically active fragment thereof.

9. The recombinant bacteriophage of embodiment 8, wherein the exogenous antimicrobial agent is an endonuclease.

10. The recombinant bacteriophage of embodiment 9, wherein the endonuclease is a Cas polypeptide or a biologically active fragment thereof.

11. The recombinant bacteriophage of embodiment 10, wherein the Cas polypeptide is selected from the group consisting of: a Type I Cas polypeptide, a Type II Cas polypeptide, a Type III Cas polypeptide, a Type IV Cas polypeptide, a Type V Cas polypeptide, and a Type VI Cas polypeptide.

12. The recombinant bacteriophage of embodiment 11, wherein the Cas polypeptide is a Type I Cas polypeptide.

13. The recombinant bacteriophage of embodiment 12, wherein the Type I polypeptide is Cas3.

14. The recombinant bacteriophage of any one of embodiments 1-13, wherein the recombinant bacteriophage further comprises a third nucleic acid sequence.

15. The recombinant bacteriophage of embodiment 14, wherein the third nucleic acid sequence comprises a spacer sequence or a crRNA that is complementary to a target nucleotide sequence in the first microbial population.

16. A method comprising contacting a population of the first microbial population and the second microbial population with the recombinant bacteriophage of any one of embodiments 1-15.

17. The method of embodiment 16, wherein the second microbial population replicates or survives at an increased rate compared to the first microbial population.

18. The method of any one of embodiments 16-17, wherein the first microbial population and the second microbial population are different strains of bacteria.

19. The method of any one of embodiments 16-18, wherein the genome of the first microbial population and the second microbial population are at least about 85% identical, 90% identical, 95% identical, 97.5% identical, or 99% identical.

20. The method of any one of embodiments 16-19, wherein the first microbial population comprises a bacterial species selected from the group consisting of E. coli, Bacteroides, Enterococcus, Shigella, Hafnia alvei, Pseudomonas aeruginosa, Morganella morganii, Pseudomonas putida, Citrobacter koseri, Klebsiella pneumonia, Campylobacter jejuni, and Mycobacterium avium subspecies paratuberculosis.

21. The method of any one of embodiments 16-20, wherein the second microbial population comprises a bacterial species selected from the group consisting of E. coli, Bacteroides, Enterococcus, Shigella, Hafnia alvei, Pseudomonas aeruginosa, Morganella morganii, Pseudomonas putida, Citrobacter koseri, Klebsiella pneumonia, and Campylobacter jejuni.

22. The method of any one of embodiments 16-20, wherein the first microbial population comprises an adherent-invasive E. coli strain and the second microbial population comprises a commensal E. coli strain.

23. The method of any one of embodiments 16-20, wherein the first microbial population comprises an E. coli strain comprising pks+sequences and the second microbial population comprises a commensal E. coli strain.

24. The method of any one of embodiments 16-20, wherein the first microbial population comprises a enterotoxigenic B. fragilis strain and the second microbial population comprises a commensal B. fragilis strain.

25. The method of any one of embodiments 16-20, wherein the first microbial population is selected from the group consisting of a shiga toxin-producing E. coli (STEC) (e.g., E. coli 0157), verocytotoxin-producing E. coli (VTEC), or enterohemorrhagic E. coli (EHEC) strains, and the second microbial population comprises a commensal E. coli strain.

26. The method of any one of embodiments 16-20, wherein the first microbial population comprises a Shigella spp. and the second microbial population comprises a commensal E. coli strain.

27. The method of any one of embodiments 16-20, wherein the first microbial population comprises a Enterococcus spp. and the second microbial population comprises a different Enterococcus spp.

28. The method of embodiment 27, wherein the first Enterococcus spp is E. faecalis or E. faecium.

29. The method of any one of embodiments 16-20, wherein the first microbial population comprises a enteropathogenic E. coli (EPEC) strain and the second microbial population comprises a commensal E. coli strain.

30. The method of any one of embodiments 16-20, wherein the first microbial population comprises a first Bacteroides spp. and the second microbial population comprises a second Bacteriodies spp.

31. The method of embodiment 30, wherein the first Bacteriodes spp. is selected from the group consisting of B. fragilis and B. thetaiotomicron.

32. The method of any one of embodiments 16-20, wherein the first microbial population comprises a species of Cutibacterium acnes that leads to acne and skin inflammation and the second microbial population comprises a species of C. acnes that promotes healthy skin.

33. The method of any one of embodiments 16-20, wherein the first microbial population comprises a microbial species found in a gut microbiome of a formula-fed infant and the second microbial population comprises a microbial species found in a gut microbiome of a breast-fed infant.

34. The method of any one of embodiments 16-20, wherein the first microbial population comprises a first species of Campylobacter jejuni associated with anxiety and the second microbial population comprises a second species of Campylobacter jejuni.

35. The method of any one of embodiments 16-20, wherein the first microbial population causes elevated levels of IgM and IgA relative to the second microbial population in a subject.

36. The method of any one of embodiments 16-35, wherein the first microbial population comprises a target sequence that is absent in the second microbial population.

37. The method of embodiment 36, wherein the target sequence comprises a sequence selected from the group consisting of an AIEC specific DNA sequence, pks+DNA sequence, a sequence encoding an enterotoxin, a sequence encoding a shiga toxin, and a sequence encoding a verocytotoxin.

38. The method of any one of embodiments 16-38, wherein the method comprises treating a disease or condition caused at least in part by the first microbial population.

39. The method of embodiment 38, wherein the disease or condition is selected from the group consisting of inflammatory bowel disease, crohn's disease, ulcerative colitis, gastrointestinal infection, gastroenteritis, dysentery, kidney failure caused by hemolytic uremic syndrome, intra-abdominal infection, food sensitization, allergies, autism, and acne.

40. The method of embodiment 38, wherein the disease or condition is a cancer.

41. The method of embodiment 40, wherein the cancer is selected from the group consisting of melanoma, non-small cell lung cancer, renal cell carcinoma, urothelial carcinoma, colorectal cancer, hepatocellular cancer, stomach cancer, large B cell lymphoma, cervical cancer, small cell lung cancer, esophageal cancer, endometrial carcinoma, cutaneous squamous cell carcinoma, breast cancer, Hodgkin lymphoma, non-Hodgkin lymphoma, head & neck cancer, Merkel cell carcinoma, microsatellite instability (MSI)-high/deficient mismatch repair (dMMR) tumors, tumor mutational burden (TMB)-high tumors, and any other tumor types approved to be treated with immunotherapies (e.g., immune checkpoint inhibitors).

42. The method of any one of embodiments 16-41, wherein the method further comprises contacting the population with an additional therapeutic.

43. The method of embodiment 42, wherein the additional therapeutic comprises an antibiotic peptide or a biologically active fragment thereof.

44. The method of embodiment 42, wherein the additional therapeutic is selected from the group consisting of oral corticosteroids, oral aminosalicylates, TNF inhibitors, immunosuppressants, and integrin/integrin receptor antagonists.

45. The method of embodiment 42, wherein the additional therapeutic is selected from the group consisting of 5-Fluorouracil, leucovorin, oxaliplatin, bevacizumab, panitumumab, cetuximab, capecitabine, irinotecan, ziv-aflibercept, ramucirumab, regorafenib, trifluridine, tipiracil, pembrolizumab, nivolumab, ipilimumab, atezolizumab, avelumab, durvalumab, cemiplimab-rwlc, trastuzumab, pertuzumab, lapatinib, encorafenib, Larotrectinib, entrectinib, and a biologically active fragment thereof.

46. The method of embodiment 42, wherein the additional therapeutic comprises carbidopa-levodopa.

47. The method of embodiment 42, wherein the additional therapeutic comprises an antidepressant.

48. The method of embodiment 42, wherein the additional therapeutic is selected from the group consisting of salicylic acid, azelaic acid, dapsone (Aczone) gel, retinoids, isotretinoin anti-androgen agents, laser therapy, chemical peels, acne extraction techniques, and steroids.

49. The method of any one of embodiments 16-48, wherein the recombinant bacteriophage is lytic; wherein (i) the recombinant lytic bacteriophage replicates within the target bacterium prior to a microbial species dying, producing a replicated recombinant lytic bacteriophage; (ii) the replicated recombinant lytic bacteriophage is released from the microbial species following death of the microbial species; and (iii) the replicated recombinant lytic bacteriophage infects a microbial species that is not infected by the recombinant lytic bacteriophage.

50. The method of any one of embodiments 16-49, wherein the bacteriophage is administered intramuscularly, intravenously, subcutaneously, orally, transmucosally, buccally, sublingually, rectally, intranasally, or topically.

51. The method of any one of embodiments 16-50, wherein the first microbial population and the second microbial population are located in a region of a body selected from the group consisting of the gastrointestinal tract, skin, intraperitoneal cavity, colon, pancreas, bile duct, liver, and gall bladder.

52. A pharmaceutical composition comprising the recombinant bacteriophage of any one of embodiments 1-16, and a pharmaceutically acceptable excipient.

### EXAMPLES

### Example 1: Selectively Killing Adherent-Invasive E. coli (AIEC) Strains Over Commensal E. coli Strains

Adherent-invasive E. coli (AIEC) strains strongly adhere to and invade intestinal epithelial cells. AIEC strains are associated with Inflammatory Bowel Disease (IBD). Reducing the population of AIEC strains in the gastrointestinal system is used to improved health in a patient with IBD.

A patient with IBD or who is likely to develop IBD is administered a phage to the gastrointestinal tract. The phage comprises a Type I CRISPR system and spacers targeting AIEC-specific sequences. In an example case, the Type I CRISPR system is a Type IF system, e.g., from E. coli (ECIF). The spacer has a sequence at least 80% identical to SEQ ID NO: 37, e.g., at least 85%, 90%, or 95% identical to SEQ ID NO: 37. In another example case, the Type I CRISPR system is a Type IC system, e.g., from P. aeruginosa (PAIC). The spacer has a sequence at least 80% identical to SEQ ID NO: 38, e.g., at least 85%, 90%, or 95% identical to SEQ ID NO: 38. In another example case, the Type I CRISPR system is a Type IE system, e.g., from E. coli (ECIE). The spacer has a sequence at least 80% identical to SEQ ID NO: 39, e.g., at least 85%, 90%, or 95% identical to SEQ ID NO: 39.

Administration of the phage with the Type I CRISPR system is intramuscular, intravenous, subcutaneous, oral, transmucosal or rectal. In an example, the patient is treated in combination with oral corticosteroids; oral aminosalicylates [e.g., mesalamine (Asacol HD, Delzicol, others), balsalazide (Colazal) and olsalazine (Dipentum)]; TNF inhibitors [e.g., Remicade (infliximab), Enbrel (etanercept), Humira (adalimumab), Cimzia (certolizumab pegol) and Simponi (golimumab)]; immunosuppressants [e.g., azathioprine (Azasan, Imuran), mercaptopurine (Purinethol, Purixan), cyclosporine (Gengraf, Neoral, Sandimmune) and methotrexate (Trexall)]; and integrin/integrin receptor antagonists [e.g., natalizumab (Tysabri), vedolizumab (Entyvio), ustekinumab (Stelara), and guselkumab (Tremfya)].

The phage is an obligately lytic bacteriophage, non-replicative, lytic bacteriophage, or non-replicative, non-lytic bacteriophage. A phage can be obligately lytic bacteriophage in that it could either be found as a lytic phage or rendered lytic through recombinant methods. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills AIEC strains over non-AIEC strains. This results in regrowth of commensal, non-AIEC strains of E. coli and/or other commensals including but not limited to Bacteroides fragilis, Bacteroides melaninogenicus, Bacteroides oralis, Enterococcus faecalis, Escherichia coli, Bifidobacterium bifidum, Staphylococcus aureus, Clostridium perfringens, Proteus mirabilis, Clostridium tetani, Clostridium septicum, Pseudomonas aeruginosa, Salmonella enterica, Faecalibacterium prausnitzii, Peptostreptococcus spp., Peptococcus spp., Lactobacillus spp., Enterobacter spp., or Klebsiella spp., over the CRISPR-targeted strains. Additional example AIEC and non-AIED strains are provided in Table 2. This improves the prognosis of patient with or likely to develop the IBD.

### Example 2: Selectively Killing Pks+E. coli Strains Over Commensal E. coli Strains

Colibactin accumulation may promote colon tumor growth. pks+E. coli strains comprise the pathogenicity island pks, which encodes a set of enzymes that synthesize colibactin. Selectively targetingpks+E. coli strains can reduce colibactin accumulation and lead to improved clinical outcomes of patients with colon cancer.

A patient with colorectal cancer is administered a phage to the gastrointestinal tract. The phage comprises a Type I CRISPR system and spacers targeting pks+-specific sequences. In an example case, the Type I CRISPR system is a Type IF system, e.g., from E. coli (ECIF). The spacer has a sequence at least 80% identical to SEQ ID NO: 40, e.g., at least 85%, 90%, or 95% identical to SEQ ID NO: 40. In another example case, the Type I CRISPR system is a Type IC system, e.g., from P. aeruginosa (PAIC). The spacer has a sequence at least 80% identical to SEQ ID NO: 41, e.g., at least 85%, 90%, or 95% identical to SEQ ID NO: 41. In another example case, the Type I CRISPR system is a Type IE system, e.g., from E. coli (ECIE). The spacer has a sequence at least 80% identical to SEQ ID NO: 42, e.g., at least 85%, 90%, or 95% identical to SEQ ID NO: 42.

Administration of the phage with the Type I CRISPR system is intramuscular, intravenous, subcutaneous, oral, transmucosal, or rectal. In an example, the patient is treated with anticancer drugs such as 5-Fluorouracil, leucovorin, oxaliplatin, bevacizumab, panitumumab, cetuximab, capecitabine, irinotecan, ziv-aflibercept, ramucirumab, regorafenib, trifluridine, tipiracil, pembrolizumab, nivolumab, ipilimumab, trastuzumab, pertuzumab, lapatinib, encorafenib, Larotrectinib, or entrectinib.

The phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills pks+E. coli strains over commensal E. coli strains. Example pks+E. coli and commensal E. coli strains are provided in Table 2. This results in regrowth of commensal, non-pks+strains of E. coli over the CRISPR targeted strains. This improves the prognosis of the patient with the colorectal cancer.

### Example 3: Selectively Killing Enterotoxigenic B. fragilis Strains Over Commensal B. fragilis Strains

Enterotoxigenic strains of B. fragilis are associated with colon cancer.

A patient with colorectal is administered a phage to the gastrointestinal tract. The phage comprises a Type I CRISPR system and spacers targeting enterotoxigenic B. fragilis sequences over non-CRISPR-targeted B. fragilis strains. In an example case, the Type I CRISPR system is a Type IF system, e.g., from E. coli (ECIF). The spacer has a sequence at least 80% identical to SEQ ID NO: 43, e.g., at least 85%, 90%, or 95% identical to SEQ ID NO: 43. In another example case, the Type I CRISPR system is a Type IC system, e.g., from P. aeruginosa (PAIC). The spacer has a sequence at least 80% identical to SEQ ID NO: 44, e.g., at least 85%, 90%, or 950% identical to SEQ ID NO: 44. In another example case, the Type I CRISPR system is a Type IE system, e.g., from E. coli (ECIE). The spacer has a sequence at least 80% identical to SEQ ID NO: 45, e.g., at least 85%, 90%, or 95% identical to SEQ ID NO: 45.

Administration of the phage with the Type I CRISPR system is intramuscular, intravenous, subcutaneous, oral, transmucosal, or rectal. In an example, the patient is treated with anticancer drugs such as 5-Fluorouracil, leucovorin, oxaliplatin, bevacizumab, panitumumab, cetuximab, capecitabine, irinotecan, ziv-aflibercept, ramucirumab, regorafenib, trifluridine, tipiracil, pembrolizumab, nivolumab, ipilimumab, trastuzumab, pertuzumab, lapatinib, encorafenib, Larotrectinib, or entrectinib.

The phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills enterotoxigenic B. fragilis strains over commensal strains. This results in regrowth of commensal non-enterotoxigenic B. fragilis strains over the CRISPR targeted strains. Non-limiting example enterotoxigenic and commensal strains are provided in Table 2. This improves the prognosis of the patient with the colorectal cancer.

### Example 4: Selectively Killing Toxin-Producing E. coli Strains

A patient with an infectious disease is administered a phage to the gastrointestinal tract. The phage comprises a Type I CRISPR system and spacers targeting toxin producing strains, such as shiga toxin-producing E. coli (STEC) (e.g., E. coli 0157), verocytotoxin-producing E. coli (VTEC), or enterohemorrhagic E. coli (EHEC) strains. In an example, the sequences are toxin-specific sequences. The infectious disease is a gastrointestinal infection, gastroenteritis, dysentery, or kidney failure caused by hemolytic uremic syndrome. Administration of the phage with the Type I CRISPR system is intramuscular, intravenous, subcutaneous, oral, transmucosal or rectal. In an example, the patient is treated with antibiotics such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, or pazufloxacin.

The phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills shiga toxin-producing E. coli (STEC) (e.g., E. coli 0157), verocytotoxin-producing E. coli (VTEC), or enterohemorrhagic E. coli (EHEC) strains over commensal E. coli strains. This results in regrowth of commensal E. coli over the CRISPR targeted strains. This improves the prognosis of the patient with the gastrointestinal infection.

### Example 5: Selectively Killing Shigella Spp. Over Non-CRISPR-Targeted E. coli Stains

A patient with an infectious disease is administered a phage to the gastrointestinal tract. The infectious disease is a gastrointestinal infection, gastroenteritis, dysentery, or kidney failure caused by hemolytic uremic syndrome. The infection may be caused by Shigella spp.

The phage comprises a Type I CRISPR system and spacers targeting Shigella spp. over non-targeted, commensal E. coli strains. In an example, the phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In one example, the phage may include sequences for anti-toxin production.

Administration of the phage with the Type I CRISPR system is intramuscular, intravenous, subcutaneous, oral, transmucosal, or rectal. In an example, the patient may also be treated with antibiotics such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, or pazufloxacin.

The phage selectively kills Shigella spp. over commensal E. coli strains. This results in regrowth of commensal E. coli over the CRISPR targeted strains. This improves the prognosis of the patient with the gastrointestinal infection.

### Example 6: Selectively Killing Target Enterococcus Spp

A patient with an intraabdominal infection (e.g., peritonitis, diverticulitis, cholecystitis, cholangitis, and pancreatitis) is administered a phage to the intraperitoneal cavity, colon, pancreas, bile duct, liver, or gallbladder. Administration is intramuscular, intravenous, subcutaneous, oral, transmucosal, or rectal. In an example, The patient is also treated with antibiotics such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, or pazufloxacin.

The phage comprises a Type I CRISPR system and spacers targeting pathogenic Enterococcus strains (e.g. E. faecalis, E. faecium) over non-CRISPR targeted strains. In an example case, the Type I CRISPR system is a Type IF system, e.g., from E. coli (ECIF). The spacer has a sequence at least 80% identical to SEQ ID NO: 46, e.g., at least 85%, 90%, or 95% identical to SEQ ID NO: 46. In another example case, the Type I CRISPR system is a Type IC system, e.g., from P. aeruginosa (PAIC). The spacer has a sequence at least 80% identical to SEQ ID NO: 47, e.g., at least 85%, 90%, or 95% identical to SEQ ID NO: 47. In another example case, the Type I CRISPR system is a Type IE system, e.g., from E. coli (ECIE). The spacer has a sequence at least 80% identical to SEQ ID NO: 48, e.g., at least 85%, 90%, or 95% identical to SEQ ID NO: 48.

The phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills targeted Enterococcus spp. over commensal strains. This results in regrowth of commensal Enterococcus spp. over the CRISPR targeted strains. Example Enterococcus spp. and commensal strains are provided in Table 2. This improves the prognosis of the patient with the intraabdominal infection.

### Example 7: Selectively Killing Enteropathogenic E. coli (EPEC) Strains Over Commensal E. coli Strains

A patient with an infectious disease (e.g. a gastrointestinal infection or gastroenteritis) or a disease of the central nervous system (e.g. Parkinson's disease) is administered a phage to the gastrointestinal tract. Administration is intramuscular, intravenous, subcutaneous, oral, transmucosal or rectal. In an example, the patient is also treated with antibiotics or carbidopa-levodopa.

The phage comprises a Type I CRISPR system and spacers targeting EPEC-specific sequences. The phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. The phage may include sequences for anti-toxin production.

The phage selectively kills EPEC over commensal E. coli strains. This results in regrowth of commensal E. coli strains over the CRISPR targeted strains. This improves the prognosis of the patient.

### Example 8: Selectively Killing Target Bacteriodes Spp Over Non-Targeted Bacterioides to Treat Infections

A patient with an intraabdominal infection (e.g. peritonitis, diverticulitis, cholecystitis, cholangitis, pancreatitis) is administered a phage to the gastrointestinal tract. Administration is intramuscular, intravenous, subcutaneous, oral, transmucosal, or rectal. In an example, the patient is also treated with antibiotics such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, or pazufloxacin.

The phage comprises a Type I CRISPR system and spacers targeting sequences specific to the targeted Bacterioides spp. The phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills selected Bacterioides strains (e.g. B. fragilis, B. thetaiotaomicron) over commensal Bacteriorides strains. This results in regrowth of commensal Bacteriodies strains over the CRISPR targeted strains. This improves the prognosis of the patient with the infection.

### Example 9: Selectively Killing Target Bacteriodes Spp Over Non-Targeted Bacterioides to Treat Cancer

A patient with a cancer or tumor is administered a phage to the gastrointestinal tract. The cancer or tumor is melanoma, non-small cell lung cancer, renal cell carcinoma, urothelial carcinoma, colorectal cancer, hepatocellular cancer, stomach cancer, large B cell lymphoma, cervical cancer, small cell lung cancer, esophageal cancer, endometrial carcinoma, cutaneous squamous cell carcinoma, breast cancer, Hodgkin lymphoma, non-Hodgkin lymphoma, head & neck cancer, Merkel cell carcinoma, microsatellite instability (MSI)-high/deficient mismatch repair (dMMR) tumors, tumor mutational burden (TMB)-high tumors, and any other tumor types approved to be treated with immunotherapies (e.g., immune checkpoint inhibitors). Administration is intramuscular, intravenous, subcutaneous, oral, transmucosal, or rectal. In an example, the patient is also treated with immune checkpoint inhibitors (e.g, pembrolizumab, nivolumab, ipilumumab, atezolizumab, avelumab, durvalumab, cemiplimab-rwlc).

The phage comprises a Type I CRISPR system and spacers targeting sequences specific to the targeted Bacterioides spp. The phage may be an obligately lytic bacteriophage, non-replicative, lytic bacteriophage, or non-replicative, non-lytic bacteriophage. The phage may be modified to result in improved biofilm degradation or have modified or swapped tail fibers for increased/decreased host range. The phage may deliver alternative phage payloads (i.e. antimicrobial nanoparticles or receptor binding proteins) that can kill phage resistant bacteria when released. The phage may include sequences for anti-toxin production.

The phage selectively kills selected Bacterioides strains (e.g. B. fragilis, B. thetaiotaomicron) over commensal Bacteriorides strains. This results in regrowth of commensal Bacteriodies strains over the CRISPR targeted strains. This improves the prognosis of the patient with the cancer or tumor.

### Example 10: Selectively Killing Strains of Cutibacterium acnes that Lead to Acne and Skin Inflammation while Enriching for Beneficial Strains of C. acnes that Promote Healthy Skin

Certain strains of C. acnes are associated with acne, while other strains of C. acnes are associated with healthy skin. A patient with acne is administered a phage to the skin.

Administration is inhaled, transdermal, topical, intramuscular, intravenous, subcutaneous, oral, or transmucosal. In an example, the patient is also treated with antibiotics, salicylic acid, azelaic acid, dapsone (Aczone) gel, retinoids, isotretinoin anti-androgen agents, laser therapy, chemical peels, acne extraction techniques, and steroids.

The phage comprises a Type I CRISPR system and spacers targeting sequences in C. acnes that lead to acne and skin inflammation. The phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills acne-causing strains of C. acnes over commensal strains of C. acnes that promote healthy skin. This results in regrowth of commensal strains of C. acnes over the CRISPR targeted strains, promoting healthy skin.

### Example 11: Selectively Killing Non-Desirable Strains of Bacteria Found in the Gut Microbiome of Formula-Fed Infants to Rebalance Commensal Community to be More Similar to Breast Milk Fed Infants

The microbiome of breast-fed infants differs from that of formula-fed infants. These differences in microbiomes are associated with developing food sensitization, food allergies, and autism. A phage is administered to the gastrointestinal tract to selectively target non-desirable strains of bacteria found in the gut microbiome of formula-fed infants. Administration is intramuscular, intravenous, subcutaneous, oral, transmucosal or rectal. In an example, the patient is also treated with antibiotics such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, or pazufloxacin.

The phage comprises a Type I CRISPR system and spacers targeting sequences specific to strains of bacteria found in the gut microbiome of formula-fed infants. In an example, the phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills species found in the microbiome of formula-fed infants. This allows for increased growth of strains found in breastmilk fed infants, rebalancing the microbiome to more closely match the microbiome of a breastmilk-fed infant.

### Example 12: Selectively Killing Strains of Campylobacter jejuni from the Gut Microbiome Associated with Anxiety Over Other Commensal Strains

Certain strains of C. jejuni are associated with anxiety, depression, and other forms of cognitive dysfunction. A patient with anxiety or likely to develop anxiety is administered a phage to the gastrointestinal tract. Administration is intramuscular, intravenous, subcutaneous, oral, transmucosal or rectal. In an example, the patient is also treated with antibiotics such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, or pazufloxacin.

The phage comprises a Type I CRISPR system and spacers targeting sequences specific to strains of C. jejuni associated with anxiety. In an example, the phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills C. jejuni strains associated with anxiety over commensal D. jejuni strains. This results in regrowth of commensal C. jejuni over the CRISPR targeted strains. This reduces anxiety or depression in the patient.

### Example 13: Selectively Killing Strains of Gut Microbiota Associated with Elevated Levels of IgM and IgA Relative to Other Commensal Strains

Depression and other forms of cognitive dysfunction are associated with elevated levels of IgM and IgA in the gut against certain microbial species (e.g. Hafnia alvei, Pseudomonas aeruginosa, Morganella morganii, Pseudomonas putida, Citrobacter koseri, and Klebsiella pneumoniae). A patient with depression or who is likely to develop depression is administered a phage to the gastrointestinal tract. Administration is intramuscular, intravenous, subcutaneous, oral, inhaled, transmucosal, or rectal. In an example, the patient is also treated with antibiotics or antidepressants.

The phage comprises a Type I CRISPR system and spacers targeting sequences specific to strains of bacteria associated with elevated levels of IgA and IgM. The phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills microbial species associated with elevated levels of IgA and IgM in a subject over commensal strains. This results in regrowth of commensal strains over the CRISPR targeted strains, resulting in decreased depression and decreased cognitive dysfunction in the patient.

### Example 14: Selectively Killing Strains of Mycobacterium avium Subspecies Paratuberculosis (MAP) Over Other Commensal Strains

A patient with Crohn's disease or who is likely to develop Crohn's disease is administered a phage to the gastrointestinal tract. Administration is intramuscular, intravenous, subcutaneous, oral, transmucosal or rectal. In an example, the patient is also treated with antibiotics such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, or pazufloxacin.

The phage comprises a Type I CRISPR system and spacers targeting strains of Mycobacterium avium subspecies paratuberculosis (MAP) over other commensal strains. The phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills MAP strains over commensal strains. This results in regrowth of commensal strains of bacteria, improving the outcome of the patient with Crohn's Disease over the CRISPR targeted strains.

### Example 15: Selective Killing of P. aeruginosa

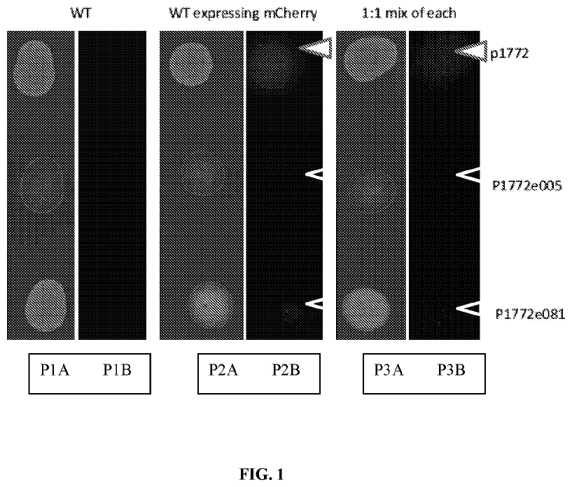

This example demonstrates that bacteriophages engineered with a CRISPR system as described herein selectively kill a target bacteria. Wildtype Pseudomonas aeruginosa b1121 or bacteria of the same strain expressing an mCherry gene (integrated into the chromosome) were treated with either a (i) wildtype phage (p1772), (ii) phage containing a Cas system targeting the bacterial genome (p1772e005), or (iii) phage containing a Cas system targeting mCherry (p1772e081) having the CRISPR array sequence shown in Table 5. The bacteria and phage were mixed at an approximate MOI of 1 and plated immediately on LB plates. The bacteria were incubated overnight at 37 C. For each pair of images, the image on the left (e.g., P1A, P2A, P3A) represents bacterial growth and the image on the right (e.g., P1B, P2B, P3B) represents viable bacteria able to express mCherry. FIG. 1 shows that the phage with the CRISPR system targeting the wildtype bacterial genome killed both the wildtype and mCherry expressing bacteria (middle rows P1, P2) and the phage that targets the mCherry were able to kill only the mCherry expressing bacteria (e.g., see P2B bottom row) and not the Wildtype bacteria (P1B, Bottom row). When the two types of bacteria were mixed 1:1 and treated with phage, the phage targeting mCherry kills the majority of mCherry containing bacteria compared to the WT phage.

In another study aimed at examining the specificity of the engineered phage in killing the bacteria, wildtype Pseudomonas aeruginosa (PA) b1121 or bacteria of the same strain but containing the mCherry gene and a carbenicillin gene integrated into the chromosome were treated with wildtype phage, phage containing a Cas system targeting the bacterial genome (p1772e005) and phage containing a Cas system targeting mCherry (p1772e081). The bacteria and phage were mixed at an approximate MOI of 1 and plated immediately on either LB or carbenicillin containing plates. The bacteria were incubated overnight at 37 C. For each pair of images, the image on the left represents bacterial growth on LB and the image on the right represents growth on LB containing carbenicillin. Upon incubation, it was seen that the phage with the Cas system targeting the wildtype bacterial genome killed both the wildtype and modified strain and the phage that targets the mCherry bacteria only kills that strain. When the two types of bacteria are mixed 1:1 and treated with phage, no carbenicillin resistant colonies were recovered.

These studies demonstrate that the engineered bacteriophage are successfully in selectively killing the bacteria.

Table 5 provides sequences for targeting mCherry sequence.

TABLE 5

| | SEQ | |
|---|---|---|
| DOMAIN/ | ID | |
| REGION | NO | SEQUENCE |
| Promoter/ | 31 | ACAAGCGGCACATTGTGCCTATTGCGAATTAGGCACAATGTGCCTAATCTA |
| RBS | | ACGTCATGCCAGCCACAACGGCGAGGCGCCAAGAAGGATAGAAGCC |
| Spacer 1 | 32 | agatagtccgggatatcggccggatgcttaacgt |
| Spacer 2 | 33 | ccgagcgatggtccggttatgcagaaaaagacga |
| Repeat | 34 | GTCGCGCCCCGCACGGGCGCGTGGATTGAAAC |
| Entire | 35 | ACAAGCGGCACATTGTGCCTATTGCGAATTAGGCACAATGTGCCTAATCT |
| array | | AACGTCATGCCAGCCACAACGGCGAGGCGCCAAGAAGGATAGAAGCCGT |
| sequence | | CGCGCCCCGCACGGGCGCGTGGATTGAAACagatagtccgggatatcggccggatgcttaa |
| | | cgtGTCGCGCCCCGCACGGGCGCGTGGATTGAAACccgagcgatggtccggttatgcaga |
| | | aaaagacgaGTCGCGCCCCGCACGGGCGCGTGGATTGAAAC |
| mCherry | 36 | TTTACAGCTAGCTCAGTCCTAGGGACTGTGCTAGCATTAAAGAGGAGAAAatggtttcaaaagg |
| Sequence | | Cgaagaagacaacatggcgattatcaaggaatttatgcgtttcaaggtccacatggaaggcagcgtcaatggtcacga |
| | | Atttgaaattgaaggcgaaggtgaaggccgtccgtatgaaggcacccagacggcaaaactgaaggtcaccaaaggcg |
| | | Gtccgctgccgtttgcttgggatattctgtcaccgcaattcatgtatggttcgaaagcgtacgttaagcatccggccgata |
| | | Tcccggactatctgaaactgtcctttccggaaggcttcaaatgggaacgtgttatgaacttcgaagatggcggtgtggtt |
| | | Accgtcacgcaggatagctctctgcaagacggtgaatttatttataaagtgaagctgcgcggcaccaatttcccgagcg |
| | | Atggtccggttatgcagaaaaagacgatgggctgggaagcgagttccgaacgtatgtacccggaagacggtgccctga |
| | | Aaggcgaaatcaagcagcgcctgaaactgaaggatggcggtcactatgacgcagaagtgaaaaccacgtacaaggct |
| | | Aaaaagccggtccaactgccgggtgcatacaacgtgaacatcaagctggatatcaccagccataacgaagactatacg |
| | | atcgttgaacagtacgaacgtgcagaaggccgccactctaccggcggtatggatgaactgtacaaataa |

In Table 5, sequences in lower case letters in the row designated “Entire Array Sequence” are the spacer sequences. In the row designated, “mCherry Sequence,” sequence shown in lower case letters are the mCherry gene sequence, and sequence shown in upper case letters are promoter and RBS sequences.

### Example 16: Selectively Killing Vancomycin-Resistant Enterococcus Spp

In this prophetic example, use of a method and compositions are described in which a patient who is scheduled for an elective abdominal surgery or a cancer patient who is to receive a course of chemotherapy/radiotherapy/immunotherapy has or is detected to have vancomycin-resistant Enterococcus spp. present within the gut microbiota. These antibiotic-resistant bacteria pose a risk for an opportunistic infection in a patient who is immunocompromised due to either surgery or their cancer treatment, both of which create an opportunity for an opportunistic pathogen to cause either an intra-abdominal infection or a bloodstream infection. By selectively removing vancomycin-resistant strains prophylactically, the patient has a significantly reduced risk of developing a vancomycin-resistant Enterococci (VRE) infection over the course of their recovery or treatment. For this purpose bacteriophage systems described in the disclosure engineered with a Type 1 CRISPR-system targeting the VREs is used as described below.

The phage comprises a Type I CRISPR system and spacers targeting vancomycin-resistant Enterococcus strains (e.g. E. faecalis, E. faecium) over non-CRISPR targeted strains. In an example case, the Type I CRISPR system is a Type IF system, e.g., from E. coli (ECIF). The spacer has a sequence at least 80% identical to SEQ ID NO: 46, e.g., at least 85%, 90%, or 95% identical to SEQ ID NO: 46. In another example case, the Type I CRISPR system is a Type IC system, e.g., from P. aeruginosa (PAIC). The spacer has a sequence at least 80% identical to SEQ ID NO: 47, e.g., at least 85%, 90%, or 95% identical to SEQ ID NO: 47. In another example case, the Type I CRISPR system is a Type IE system, e.g., from E. coli (ECIE). The spacer has a sequence at least 80% identical to SEQ ID NO: 48, e.g., at least 85%, 90%, or 95% identical to SEQ ID NO: 48.

The phage is an obligately lytic bacteriophage, non-replicative, lytic bacteriophage, or non-replicative, non-lytic bacteriophage. In an example, the phage is modified to result in improved biofilm degradation or have modified or swapped tail fibers for increased/decreased host range. In an example, the phage delivers alternative phage payloads (i.e. antimicrobial nanoparticles or receptor binding proteins) that can kill phage resistant bacteria when released. In an example, the phage includes sequences for anti-toxin production.

The phage selectively kills targeted vancomycin-resistant Enterococcus spp. over commensal strains. This results in regrowth of commensal Enterococcus spp. over the CRISPR targeted strains. Example Enterococcus spp. and commensal strains are provided in Table 2. This improves the prognosis of the patient and reduces the likelihood that they develop a VRE infection.

While preferred embodiments of the present disclosures have been shown and described herein, it will be obvious to those skilled in the art that such embodiments are provided by way of example only. Numerous variations, changes, and substitutions will now occur to those skilled in the art without departing from the disclosures. It should be understood that various alternatives to the embodiments of the disclosures described herein may be employed in practicing the disclosures. It is intended that the following claims define the scope of the disclosures and that methods and structures within the scope of these claims and their equivalents be covered thereby.

What is claimed is:

1. A recombinant bacteriophage comprising an exogenous antimicrobial agent specific for a first microbe, wherein
(a) the first microbe comprises a first microbe of Table 2, column 1, and/or
(b) the first microbe comprises a target DNA sequence at least 10 nucleobases in length and
(i) at least 80% identical to a target DNA of Table 2, column 3 and/or 4, and/or (ii) at least 80% identical to any one of SEQ ID NOS: 37-48.

2. The recombinant bacteriophage of claim 1, wherein the first microbe comprises the first microbe of Table 2, column 1.

3. The recombinant bacteriophage of claim 1 or claim 2, wherein the first microbe comprises the target DNA sequence at least 10 nucleobases in length and at least 80% identical to a target DNA of Table 2, column 3 and/or 4.

4. The recombinant bacteriophage of any one of claims 1-3, wherein the first microbe comprises the target DNA sequence at least 10 nucleobases in length and at least 80% identical to a target DNA of Table 3.

5. The recombinant bacteriophage of any one of claims 1-4, wherein the antimicrobial agent comprises one or more components of a CRISPR system.

6. The recombinant bacteriophage of claim 5, wherein the CRISPR system and the antimicrobial agent comprise a CRISPR array comprising a spacer sequence at least 80% identical to any one of SEQ ID NOS: 37-48.

7. The recombinant bacteriophage of claim 5 or claim 6, wherein the CRISPR system and the antimicrobial agent comprise a CRISPR array comprising a spacer sequence at least 80% identical to at least 10 contiguous nucleobases of the target DNA of Table 2, column 3 and/or 4.

8. The recombinant bacteriophage of any one of claims 5-7, wherein the CRISPR system and the antimicrobial agent comprise a nucleic acid encoding a CRISPR nuclease.

9. The recombinant bacteriophage of claim 8, wherein the CRISPR nuclease comprises Cas3, Cas3′ and Cas3″, Cpf1, or Cas9.

10. The recombinant bacteriophage of any one of claims 5-9, wherein the CRISPR system comprises a Type I CRISPR-Cas system, a Type II CRISPR-Cas system, or a Type V CRISPR-Cas system.

11. The recombinant bacteriophage of claim 10, wherein the CRISPR system comprises the Type I CRISPR-Cas system.

12. The recombinant bacteriophage of claim 11, wherein the Type I CRISPR-Cas system comprises Cas3.

13. The recombinant bacteriophage of claim 11 or claim 12, wherein the Type I CRISPR-Cas system is a Type I-A CRISPR-Cas system, a Type I-B CRISPR-Cas system, a Type I-C CRISPR-Cas system, a Type I-D CRISPR-Cas system, a Type I-E CRISPR-Cas system, or a Type I-F CRISPR-Cas system.

14. The recombinant bacteriophage of claim 13, wherein the Type I CRISPR-Cas system is an E. coli Type I-F system (e.g., ECIF).

15. The recombinant bacteriophage of claim 13, wherein the Type I CRISPR-Cas system is an E. coli Type I-E system (e.g., ECIE).

16. The recombinant bacteriophage of claim 13, wherein the Type I CRISPR-Cas system is a P. aeruginosa Type 1-C system (e.g., PAIC).

17. The recombinant bacteriophage of any one of claims 1-16, prepared by a method comprising introducing into a first bacteriophage the exogeneous antimicrobial agent.

18. The recombinant bacteriophage of claim 17, wherein the first bacteriophage is a P1 phage, a M13 phage, a λ phage, a T4 phage, a T7 phage, a T7m phage, a φC2 phage, a φCD27 phage, a φNM1 phage, a Bc431 v3 phage, a φ10 phage, a φ25 phage, a φ151 phage, a A511-like phage, a B054, a 0176-like phage, a Campylobacter phage, p004k (PTA-127149), p00c0 (PTA-127143), p00ex (PTA-127145), p00jc (PTA-127147), p00ke (PTA-127148), p5516 (PTA-127151), p0046-9, p0033s-6, p0071-16, p0033L-10, p00ex-2, p0031-8, p004k-5, p0045-9, p0078-4, p00dd-1, p00E8-3, p00Jc-2, p006008, p006009, p006010, p006012, p006013, p006016, p006018, p006071, p006072, p006098, p006099, p006128, p006129, p5852, p5853, p3854-40-8, p3855-56-3, p4075, p4076, p4077, p4078, p4079, p4082, p4083, p4084, p4085, p4087, p4088, p4090, p4092, p4093, p4094, p5097, p5496, p5497, p5499, p5501, p5503, p5505, p5506, p5507, p5508, p5509, p5511, p5512, or any unmodified bacteriophage.

19. The recombinant bacteriophage of any one of claims 1-18, wherein the recombinant bacteriophage is an obligately lytic bacteriophage.

20. The recombinant bacteriophage of any one of claims 1-19, wherein the recombinant bacteriophage targets the first or the first microbe and a second microbe, wherein the first microbe is a microbe genus, or a microbe species or a microbe sub-species.

21. The recombinant bacteriophage of claim 20, wherein the microbe species is E. coli, B. fragilis or Enterococcus sp.

22. The recombinant bacteriophage of any one of claims 1-21, wherein the exogenous antimicrobial agent is not specific for a second microbe.

23. The recombinant bacteriophage of claim 22, wherein the first microbe comprises the target DNA sequence and the second microbe does not comprise the target DNA sequence.

24. The recombinant bacteriophage of claim 22 or claim 23, wherein the exogeneous antimicrobial agent selectively kills the first microbe and not the second microbe.

25. The recombinant bacteriophage of any one of claims 22-24, wherein the recombinant bacteriophage is capable of infecting the first microbe and the second microbe.

26. The recombinant bacteriophage of any one of claims 22-25, wherein the second microbe is a second microbe of Table 2, column 6.

27. The recombinant bacteriophage of any one of claims 22-26, wherein the first microbe comprises an adherent-invasive E. coli and the second microbe comprises a commensal E. coli.

28. The recombinant bacteriophage of any one of claims 22-26, wherein the first microbe comprises a pks+E. coli and the second microbe comprises a commensal E. coli.

29. The recombinant bacteriophage of any one of claims 20-24, wherein the first microbe comprises enterotoxigenic B. fragilis and the second microbe comprises a commensal B. fragilis.

30. The recombinant bacteriophage of any one of claims 22-26, wherein the first microbe comprises a vancomycin resistant Enterococcus (VRE) and the second microbe comprises a vancomycin sensitive Enterococcus.

31. The recombinant bacteriophage of any one of claims 20-30, wherein
(a) the first and the second microbe are targeted by the bacteriophage, and
(b) the exogenous antimicrobial agent preferentially targets the first microbe over the second microbe.

32. A method of microbial killing or restricting the expansion of a microbial population, the method comprising combining the recombinant bacteriophage of any one of claims 1-31 with the first microbe of any one of claims 1-31.

33. A method of selective microbial killing or selectively restricting the expansion of a microbial population, the method comprising combining the recombinant bacteriophage of any one of claims 20-31 with the first and second microbe of any one of claims 20-31, wherein the first microbe is killed by the recombinant bacteriophage at a higher efficiency relative to the second microbe; and/or wherein expansion of the first microbe is restricted by the bacteriophage relative to expansion of the second microbe.

34. The method of claim 33, wherein the first microbe is killed by the recombinant bacteriophage and the second microbe is not killed by the recombinant bacteriophage; and/or wherein the expansion of the first microbe is restricted by the recombinant bacteriophage and the expansion of the second microbe is not restricted by the recombinant bacteriophage.

35. A method of treating or preventing a disease or condition in a subject in need thereof, the method comprising administering to the subject the recombinant bacteriophage of claim 27, wherein disease or condition is inflammatory bowel disease and/or colorectal cancer.

36. A method of treating or preventing a disease or condition in a subject in need thereof, the method comprising administering to the subject the recombinant bacteriophage of claim 28, wherein disease or condition is inflammatory bowel disease.

37. A method of treating or preventing a disease or condition in a subject in need thereof, the method comprising administering to the subject the recombinant bacteriophage of claim 28, wherein disease or condition is colon cancer.

38. A method of treating or preventing a disease or condition in a subject in need thereof, the method comprising administering to the subject the recombinant bacteriophage of claim 29, wherein disease or condition is inflammatory bowel disease and/or colon cancer.

39. A method of treating or preventing a disease or condition in a subject in need thereof, the method comprising administering to the subject the recombinant bacteriophage of claim 30, wherein disease or condition is an opportunistic bacterial infection caused by vancomycin-resistant enterococci (VRE) in patients undergoing chemotherapy or surgery or in patients at high risk of developing a VRE infection.
